SEQUENCE LISTING (30 sequences)

SEQ ID NO: 1 - protein, 238 aa, Artificial Sequence
  PfEMMA1 fragment
DNVNNNNNKESCDNIKHMRTKSLNFVSRESYGEHKSLDVYQECYVKNNKLINKVNDKKYEDNNNSYLNEDDNASMQFYEETNSNPYIVDQENNMKNYVNNVLYNNNSNYYVDSKNYDKSKENAENKSDDILNNENIHTLKDQKKKIQNNNEFISEQADIENIRNSQEEVYEKEHEPLWVINASNEEKKSYEELIYSDMSSNRVTKNKYSDMNNVEVLLNEDNLLTTEKYKVQLEKENK

SEQ ID NO: 2 - DNA, 714 bp, Artificial Sequence
  Frag1 nucleotide fragment
gataatgttaataataataataataaagaaagttgtgataatattaaacatatgagaacaaaaagtttaaattttgtaagtagagaatcctatggcgaacataaaagtctagatgtttaccaggaatgttatgtaaaaaataataaacttattaataaggtaaatgataaaaaatatgaggacaataataattcctatcttaatgaagatgataacgctagtatgcaattttatgaagaaactaatagtaatccatatattgtagaccaggaaaataatatgaaaaattatgtcaataatgttttatataacaacaatagcaattattatgttgattcaaagaattatgataaatctaaagagaatgcagaaaataaatcagatgatatattaaataatgaaaatatacataccttaaaagatcaaaaaaagaaaatacaaaataataatgaattcattagtgaacaggctgatatagaaaatataagaaattctcaagaagaagtatatgagaaagaacacgaacctttgtgggtaataaatgcatctaatgaagaaaagaaatcatatgaagaattgatatacagcgatatgtcatctaatcgtgttacgaaaaataaatatagtgatatgaataatgttgaggtattattaaatgaagataatttattaactactgaaaaatacaaggtgcaattagaaaaagaaaataaa

SEQ ID NO: 3 - protein, 237 aa, Artificial Sequence
  PfEMMA1 fragment
NRVTKNKYSDMNNVEVLLNEDNLLTTEKYKVQLEKENKMIDMYETVEENINTIKTENTNDINEEVRNEQKRESINHINDTNINHIIDEYPNDTYNFIKDIECVHNNENNMYNSIEQYTFYHDTRNNHLVDKNNQNFIFEEEGLNELNFEEKKVYIENNTKDDHKGDSKTSNLTSLRNTICKSENDHNEKNENTYVVRKGEKGIKRKVSMKKRNEKLNEENYINNIYDKMDNHRQNDI

SEQ ID NO: 4 - DNA, 711 bp, Artificial Sequence
  Frag2 nucleotide fragment
aatcgtgttacgaaaaataaatatagtgatatgaataatgttgaggtattattaaatgaagataatttattaactactgaaaaatacaaggtgcaattagaaaaagaaaataaaatgattgatatgtatgaaacggtagaggagaatataaatacaattaaaacagaaaatacgaacgacataaatgaagaagttagaaacgaacaaaaaagagaaagtatcaatcatattaatgatacaaatataaatcatataatagatgaatatcccaatgatacatataatttcataaaagatatagaatgtgtacataacaatgaaaataacatgtacaattctattgaacaatatacattttatcatgatacacgtaataatcatttagttgataaaaataatcaaaattttatattcgaagaggaaggtttaaatgaattgaactttgaagaaaaaaaggtatatatagaaaataataccaaggatgatcacaagggagatagcaaaacaagtaacttaacatctttaaggaataccatatgtaaaagtgaaaacgatcataatgaaaaaaatgaaaacacatatgtggttagaaaaggcgaaaaaggaattaaacgtaaggtttccatgaagaaaagaaatgaaaagctaaatgaagaaaattatattaataatatatacgataaaatggataaccatagacaaaatgatatt

SEQ ID NO: 5 - protein, 84 aa, Artificial Sequence
  PfEMMA fragment
LKINKKVPKYKLENMNINSPNIPYTYLFICDGSNYLCINDNSLNNEVYENKMKLNNIIGYYHYINLNRLTYYLEKVNANFVYNH

SEQ ID NO: 6 - DNA, 252 bp, Artificial Sequence
  C-Term nucleotide
ttaaagataaataagaaagttcctaaatataaattggaaaatatgaatattaactcaccaaatattccatatacatatctttttatatgtgatggaagtaactatttatgtattaatgacaattcattaaataacgaggtatatgaaaacaagatgaaattgaacaatatcattggatattaccattatattaatttgaatagattaacatattatttagaaaaggtaaatgctaattttgtttataaccat

SEQ ID NO: 7 - protein, 42 aa, Artificial Sequence
  antigenic fragment of Frag1
DNVNNNNNKESCDNIKHMRTKSLNFVSRESYGEHKSLDVYQE

SEQ ID NO: 8 - protein, 41 aa, Artificial Sequence
  antigenic fragment of Frag1
CYVKNNKLINKVNDKKYEDNNNSYLNEDDNASMQFYEETNS

SEQ ID NO: 9 - protein, 41 aa, Artificial Sequence
  antigenic fragment of frag1
NPYIVDQENNMKNYVNNVLYNNNSNYYVDSKNYDKSKENAE

SEQ ID NO: 10 - protein, 44 aa, Artificial Sequence
  antigenic fragment of frag1
NKSDDILNNENIHTLKDQKKKIQNNNEFISEQADIENIRNSQEE

SEQ ID NO: 11 - protein, 42 aa, Artificial Sequence
  antigenic fragment of frag1
VYEKEHEPLWVINASNEEKKSYEELIYSDMSSNRVTKNKYSD

SEQ ID NO: 12 - protein, 28 aa, Artificial Sequence
  antigenic fragment of frag1
MNNVEVLLNEDNLLTTEKYKVQLEKENK

SEQ ID NO: 13 - protein, 41 aa, Artificial Sequence
  antigenic fragment of frag2
NRVTKNKYSDMNNVEVLLNEDNLLTTEKYKVQLEKENKMID

SEQ ID NO: 14 - protein, 45 aa, Artificial Sequence
  antigenic fragment of frag2
MYETVEENINTIKTENTNDINEEVRNEQKRESINHINDTNINHII

SEQ ID NO: 15 - protein, 42 aa, Artificial Sequence
  antigenic fragment of frag2
DEYPNDTYNFIKDIECVHNNENNMYNSIEQYTFYHDTRNNHL

SEQ ID NO: 16 - protein, 43 aa, Artificial Sequence
  antigenic fragment of frag2
VDKNNQNFIFEEEGLNELNFEEKKVYIENNTKDDHKGDSKTSN

SEQ ID NO: 17 - protein, 42 aa, Artificial Sequence
  antigenic fragment of frag2
LTSLRNTICKSENDHNEKNENTYVVRKGEKGIKRKVSMKKRN

SEQ ID NO: 18 - protein, 24 aa, Artificial Sequence
  antigenic fragment of frag2
EKLNEENYINNIYDKMDNHRQNDI

SEQ ID NO: 19 - protein, 44 aa, Artificial Sequence
  antigenic fragment of c-term
LKINKKVPKYKLENMNINSPNIPYTYLFICDGSNYLCINDNSLN

SEQ ID NO: 20 - protein, 40 aa, Artificial Sequence
  antigenic fragment of C-term
NEVYENKMKLNNIIGYYHYINLNRLTYYLEKVNANFVYNH

SEQ ID NO: 21 - protein, 2227 aa, Artificial Sequence
  PfEMMA1
MRSKSISYFLFFKKNKKKNDSCDSVIISSNKNLSIQLSKGEDDEKNEINEEKSYIKNEDVYKKEKLKKKKENKENNKKKDKNEVVYDYHDISNDATSDYVNNYKVYEMNTCNIKKKRESFFKKINILQKYKNYKIRKAASTFHTIGHKTSFSGTDDEIENNQKKQKKYKIKISEWKDDKSHTFHKKNDILVFDKMDKNKKFKIDNNKNNQINIDNEERVNKNYPMATNVQNFNIKYTSIDVTNDEYIIDSNKPEGSIMSTDKKNNKLNYNNDTYDVDKSSDINKLGNIKKNKFDIITKTTHNINNNVNNIHNYMMYTNKENIKININHGNLNGREQNNYDEERKANVYEIFENAKKLEPNNININTEEHIHISEPSIPFDMKDHKNDINEKDIILKLMYNNNGIYFDDDDENHKNLLYKNKDTHVKHLNNKFNHNFIIYNDREEGVNQKHAQKKLKKKNTILNKNENEDINHNSFKRPLSNTNICYKDKDDKIKNGSNKYDILNNDYSNEHEKNKYNDHITKNKRNQSANEVKSNNNDNHNNKKNNNFNININDSYSTNINRNQNVMINDVNDVIKDPNMQENTQGDDEGGIINKYLINPIYNLFLRANEEIQNSNSTNNKLKMNNITKSYTNELQKTYKSMYDINDISNKRKINNKDIRGTNLYNTKLCNNKLYNSNPYNMIPYNINTYNNNNNNKETCTSINIKHSENKYPFNKSHVNSYMKNTNHLPHRNAITSNNRNNEEYEKEKEKDRNITNGNNNYLVEYNNSCIPPPLKKMIPIDGVRNKSINKLNNVTNTQRTSSVSYTNKNIDENSFDMPIINGIRESKYISNNNNINGNSIGFNSSKLDNYHHQSMNVNESYPLKNMMKNNYIEHNYDDKNNIFLVKNYEDTYSNIHNGIHENSMLKNYNLKKACTFHGYSRNHQKNMYTEENLNINQKKNYSHYHNNGTVLKPLVNTNNVAVNEFADINLSAQKRLHSLKSMGYEDKSMENYRNKIYNNINNNNNNNNDNNIYNDNEYCQYNNSYCFDHSDLKNMFPLNHQNSKLLTHSNNKNSFFNGINVESKHHLANPEIKTFAHNSYPILNQGLINCNPLQCLGYDSNQRNKHNVVYIKKNEYLNKNIGSIINVLKREGLRKISTHNGKFESFSNMDNKNVYMEGLNIQDNVNNNNNKESCDNIKHMRTKSLNFVSRESYGEHKSLDVYQECYVKNNKLINKVNDKKYEDNNNSYLNEDDNASMQFYEETNSNPYIVDQENNMKNYVNNVLYNNNSNYYVDSKNYDKSKENAENKSDDILNNENIHTLKDQKKKIQNNNEFISEQADIENIRNSQEEVYEKEHEPLWVINASNEEKKSYEELIYSDMSSNRVTKNKYSDMNNVEVLLNEDNLLTTEKYKVQLEKENKMIDMYETVEENINTIKTENTNDINEEVRNEQKRESINHINDTNINHIIDEYPNDTYNFIKDIECVHNNENNMYNSIEQYTFYHDTRNNHLVDKNNQNFIFEEEGLNELNFEEKKVYIENNTKDDHKGDSKTSNLTSLRNTICKSENDHNEKNENTYVVRKGEKGIKRKVSMKKRNEKLNEENYINNIYDKMDNHRQNDITKKENDEENYILYNNVKVNYDEYIENGNKIKITEESLNVFYKENQNEEDSSTKKLNSTSKIKRANKGKTKKKNVITRVHKTKQKIEYVTNSFNKSSKGENSEIGKIGGRSKSLLTHSKKVSERNKNKIEKINDTNSKIIKGKKSNSQSKLGKDTKIRGKSKTGEYIKNKDLRKKSNEKNKTVMDNINTINNSSVSNLKSKKHKLKKKKKKNISMENINKNITNEFCSMERKGTVLLSNMSIKKIDNANSCTLNEPLEENTLNYESNNNCSNSNLSKDKEKDRNILCNKYYSDEETNSLNKMYTSNIPEISNYYKEIQAINYILSNINNPNFLNSLELNDLINIEKKFINENIYINKQIIACNVKNEKSNDEMVEKNERKVDEEKGEDEQEIKAKENNNKEENQDNENNNKEENHDNENNNKEENQDNENNNKEENQDNENNNKEENQDNENNNKEENQKNENGIIYDSRFSIIYLEHDLIYLKKNNLKVILNVLLSNVYCFFEIKLTIILLNFFISNNCQWSFSLFPLSLINKLIHKFSLKINKKVPKYKLENMNINSPNIPYTYLFICDGSNYLCINDNSLNNEVYENKMKLNNIIGYYHYINLNRLTYYLEKVNANFVYNHHIYE

SEQ ID NO: 22 - DNA, 6684 bp, Artificial Sequence
  PfEMMA1 nucleotide
atgagatcgaaatccatttcgtatttcttattttttaaaaaaaacaaaaagaaaaatgattcttgtgatagtgtcataatatctagcaataagaatttatccattcaattatcgaaaggtgaggatgatgaaaaaaatgaaataaatgaggaaaagagttatataaaaaatgaagatgtatataaaaaggaaaaattaaaaaagaagaaagaaaacaaggaaaataataaaaagaaagataaaaatgaagtagtatatgattatcatgacatttcaaatgatgctactagtgattatgttaataattataaagtatatgaaatgaatacttgtaatataaaaaagaagagagaaagtttttttaaaaaaattaatattttacaaaaatataaaaattacaaaattagaaaggcagctagtacctttcataccataggacataaaacatctttttctggtacagatgatgaaatagaaaataatcaaaagaaacaaaaaaaatataaaataaaaatttctgaatggaaggatgataaatcacatacttttcataaaaaaaatgacatattggtatttgataagatggataaaaataaaaaatttaaaattgataacaacaaaaacaatcaaattaatatagataatgaagaaagagttaataaaaattatcctatggctactaatgtacaaaattttaatataaaatatacatcaatagatgtaacaaatgacgaatatattatagattctaataaacctgaaggttctattatgtctacagataaaaagaataataaacttaattataataatgatacatatgatgtagacaaaagctctgatataaataagttaggtaatataaaaaagaataaatttgatattattactaaaacaacacataatattaataataatgtaaataatatacataattatatgatgtatacaaataaagaaaatataaaaataaatataaatcatggaaatctaaatggaagagaacaaaacaattatgatgaagaaaggaaagcaaatgtttatgaaatatttgaaaatgcaaaaaaattagaacctaataatattaatatcaacacagaagaacatattcatattagtgaacccagcataccatttgatatgaaggatcataaaaatgatataaatgaaaaagatataatattaaaattgatgtataacaataacggtatttattttgatgatgatgatgaaaatcacaagaatttattatacaaaaataaagatacacatgtaaaacatttaaataataaatttaaccataattttattatatataatgatcgcgaagaaggggtaaatcagaaacacgcacaaaaaaaattaaaaaaaaaaaatactattcttaacaaaaacgaaaatgaagatattaatcataatagtttcaaaagacctttatctaatacgaatatatgttataaggacaaagatgataaaattaaaaatggttctaataagtatgatatattaaataatgactattctaatgaacacgaaaaaaataaatataatgatcatataacaaaaaataaaagaaatcaatcagcaaatgaagtaaaatctaataataatgataaccacaataataaaaaaaataataattttaatattaatattaatgattcatattctacaaatataaatagaaaccaaaatgtgatgataaatgatgtaaacgatgttattaaggatccaaatatgcaggaaaatacacaaggtgatgacgaaggtggtattataaacaaatatttaattaaccctatttacaatttatttctacgtgctaatgaagaaatacaaaattcaaatagtacaaacaataaattaaaaatgaataatataacaaaaagttatacaaacgaactacaaaagacatataaaagtatgtacgatataaatgatatatcaaataagagaaaaattaataataaagatatacgtggaactaatttgtataacaccaaattatgtaataataaattatataattcgaatccatataatatgattccatataatataaacacatataataataataataataataaggaaacttgtaccagcataaatatcaaacattccgaaaataaatatcccttcaataaatctcatgtaaactcatatatgaaaaatacaaatcatcttcctcatagaaatgcgattacatcaaataatagaaacaatgaagaatatgagaaagaaaaagaaaaagatcgtaacattactaatgggaacaataattatttggttgaatataataattcttgtatacctccaccactcaaaaaaatgataccaatagatggtgtgagaaataaaagtataaataaattaaataatgtaactaatacgcaacgtacatcaagtgtttcatatacgaataagaatattgatgagaattcgtttgatatgcctataataaatggaataagagaatctaaatatataagtaataataataatattaatggtaattccattggttttaattcatctaagttagataattatcatcaccaatctatgaatgtgaatgaatcttatcctctaaaaaatatgatgaaaaataattatattgaacataattatgatgataaaaataatattttccttgttaaaaattatgaagatacatattcaaatattcataatggcatacatgaaaatagcatgctaaaaaattataatttaaaaaaagcgtgcacttttcatgggtactctagaaatcaccaaaaaaatatgtatacggaagaaaatttaaatattaatcaaaaaaagaattatagtcattatcataataatggaacggtattaaaacctttggtaaatactaataatgttgcagtgaacgaatttgcagatattaatttatcggctcaaaaaagattacatagtttaaaaagtatggggtacgaggataagagtatggaaaattacagaaacaaaatatacaacaacatcaataataataataataataataatgataataatatatataatgataatgaatattgtcagtataataatagttattgtttcgatcatagtgatttaaaaaatatgtttccattaaatcatcagaatagcaagttattaacacatagtaataataaaaattcattttttaacggaataaatgtagaatcgaaacatcatttagcaaatcctgaaataaaaacatttgcacacaatagttatcctatattaaatcaaggtttaataaattgtaaccccttacaatgcttgggttatgattcaaatcaaaggaataagcataatgtagtatacataaaaaaaaatgaataccttaataaaaacattggctctattataaatgttcttaaaagagaaggactaagaaaaatttctacacataatggaaaattcgaatcatttagtaatatggataataaaaatgtatatatggaaggactaaacatacaagataatgttaataataataataataaagaaagttgtgataatattaaacatatgagaacaaaaagtttaaattttgtaagtagagaatcctatggcgaacataaaagtctagatgtttaccaggaatgttatgtaaaaaataataaacttattaataaggtaaatgataaaaaatatgaggacaataataattcctatcttaatgaagatgataacgctagtatgcaattttatgaagaaactaatagtaatccatatattgtagaccaggaaaataatatgaaaaattatgtcaataatgttttatataacaacaatagcaattattatgttgattcaaagaattatgataaatctaaagagaatgcagaaaataaatcagatgatatattaaataatgaaaatatacataccttaaaagatcaaaaaaagaaaatacaaaataataatgaattcattagtgaacaggctgatatagaaaatataagaaattctcaagaagaagtatatgagaaagaacacgaacctttgtgggtaataaatgcatctaatgaagaaaagaaatcatatgaagaattgatatacagcgatatgtcatctaatcgtgttacgaaaaataaatatagtgatatgaataatgttgaggtattattaaatgaagataatttattaactactgaaaaatacaaggtgcaattagaaaaagaaaataaaatgattgatatgtatgaaacggtagaggagaatataaatacaattaaaacagaaaatacgaacgacataaatgaagaagttagaaacgaacaaaaaagagaaagtatcaatcatattaatgatacaaatataaatcatataatagatgaatatcccaatgatacatataatttcataaaagatatagaatgtgtacataacaatgaaaataacatgtacaattctattgaacaatatacattttatcatgatacacgtaataatcatttagttgataaaaataatcaaaattttatattcgaagaggaaggtttaaatgaattgaactttgaagaaaaaaaggtatatatagaaaataataccaaggatgatcacaagggagatagcaaaacaagtaacttaacatctttaaggaataccatatgtaaaagtgaaaacgatcataatgaaaaaaatgaaaacacatatgtggttagaaaaggcgaaaaaggaattaaacgtaaggtttccatgaagaaaagaaatgaaaagctaaatgaagaaaattatattaataatatatacgataaaatggataaccatagacaaaatgatattacaaaaaaagaaaatgacgaagaaaattatattttgtacaacaacgtaaaggttaattatgatgaatatatagaaaatggaaataaaataaaaataacggaagaatcattaaatgtcttttataaagaaaatcaaaatgaggaagattcttctacaaaaaagttgaatagtacaagtaaaataaaacgtgcaaacaaagggaaaacaaaaaaaaagaatgttatcacaagggtacataaaacaaaacaaaaaattgaatatgttacaaatagttttaataaatcttccaaaggtgaaaattcagaaataggaaaaattggaggtaggagtaaatcattattaacacacagcaagaaagttagtgaacgaaataaaaataaaatagaaaaaattaatgatacaaattcaaagataataaaaggaaaaaagagtaatagccaaagcaaacttgggaaggatacaaaaattagagggaaatcaaaaactggggaatatataaaaaataaagatttaagaaaaaaatctaacgaaaaaaacaaaacagtgatggataatataaatactataaataattcttcagtatctaacctaaaaagcaaaaaacataaattgaaaaaaaaaaaaaaaaaaaatatatctatggaaaatataaataaaaatataacaaatgaattttgttctatggaaagaaaaggaaccgttctattatctaatatgagtattaagaagattgataatgcaaatagttgtacattaaatgaaccattagaggaaaataccttaaattatgaaagtaataataactgtagtaatagtaatttatctaaggataaagaaaaagatagaaatatattgtgtaataaatattatagtgatgaggaaacaaactctttaaacaaaatgtatacatcgaatataccagaaataagtaattattataaggaaattcaagcaattaattacatattaagtaatattaataatccaaattttttaaattccctcgaactgaatgatttaataaatattgaaaaaaaatttattaacgaaaatatatatattaataagcagataatagcctgtaatgtaaaaaatgaaaaatcaaatgatgagatggtcgagaaaaatgaacgcaaagtggatgaagaaaaaggagaagacgaacaagaaataaaagcaaaggaaaataataataaagaagaaaaccaagataatgaaaataataataaagaagaaaaccatgataatgaaaataataataaagaagaaaatcaagataatgaaaataataataaagaagaaaaccaagataatgaaaataataataaagaagaaaatcaagataatgaaaataataataaagaagaaaaccaaaaaaatgaaaatggtattatttatgatagcaggtttagtattatctatttagaacacgatttaatatatttaaaaaaaaataatttaaaagtgatacttaatgttttgctgtcaaatgtgtattgcttttttgaaattaaattaaccataatattgttaaatttctttatatctaataattgtcaatggagtttcagtttatttcccctttcattaattaataaattaatacataaattcagtttaaagataaataagaaagttcctaaatataaattggaaaatatgaatattaactcaccaaatattccatatacatatctttttatatgtgatggaagtaactatttatgtattaatgacaattcattaaataacgaggtatatgaaaacaagatgaaattgaacaatatcattggatattaccattatattaatttgaatagattaacatattatttagaaaaggtaaatgctaattttgtttataaccatcatatatatgaataa

SEQ ID NO: 23 - protein, 277 aa, Artificial Sequence
  Nterm frag1 of PfEMMA1
MRSKSISYFLFFKKNKKKNDSCDSVIISSNKNLSIQLSKGEDDEKNEINEEKSYIKNEDVYKKEKLKKKKENKENNKKKDKNEVVYDYHDISNDATSDYVNNYKVYEMNTCNIKKKRESFFKKINILQKYKNYKIRKAASTFHTIGHKTSFSGTDDEIENNQKKQKKYKIKISEWKDDKSHTFHKKNDILVFDKMDKNKKFKIDNNKNNQINIDNEERVNKNYPMATNVQNFNIKYTSIDVTNDEYIIDSNKPEGSIMSTDKKNNKLNYNNDTYDVD

SEQ ID NO: 24 - protein, 277 aa, Artificial Sequence
  N-terminal fragment 2 of PfEMMA1
KSSDINKLGNIKKNKFDIITKTTHNINNNVNNIHNYMMYTNKENIKININHGNLNGREQNNYDEERKANVYEIFENAKKLEPNNININTEEHIHISEPSIPFDMKDHKNDINEKDIILKLMYNNNGIYFDDDDENHKNLLYKNKDTHVKHLNNKFNHNFIIYNDREEGVNQKHAQKKLKKKNTILNKNENEDINHNSFKRPLSNTNICYKDKDDKIKNGSNKYDILNNDYSNEHEKNKYNDHITKNKRNQSANEVKSNNNDNHNNKKNNNFNININD

SEQ ID NO: 25 - protein, 281 aa, Artificial Sequence
  N-terminal fragment 3 pf PfEMMA1
SYSTNINRNQNVMINDVNDVIKDPNMQENTQGDDEGGIINKYLINPIYNLFLRANEEIQNSNSTNNKLKMNNITKSYTNELQKTYKSMYDINDISNKRKINNKDIRGTNLYNTKLCNNKLYNSNPYNMIPYNINTYNNNNNNKETCTSINIKHSENKYPFNKSHVNSYMKNTNHLPHRNAITSNNRNNEEYEKEKEKDRNITNGNNNYLVEYNNSCIPPPLKKMIPIDGVRNKSINKLNNVTNTQRTSSVSYTNKNIDENSFDMPIINGIRESKYISNNNN

SEQ ID NO: 26 - protein, 328 aa, Artificial Sequence
  N-terminal fragment 4 of PfEMMA1
INGNSIGFNSSKLDNYHHQSMNVNESYPLKNMMKNNYIEHNYDDKNNIFLVKNYEDTYSNIHNGIHENSMLKNYNLKKACTFHGYSRNHQKNMYTEENLNINQKKNYSHYHNNGTVLKPLVNTNNVAVNEFADINLSAQKRLHSLKSMGYEDKSMENYRNKIYNNINNNNNNNNDNNIYNDNEYCQYNNSYCFDHSDLKNMFPLNHQNSKLLTHSNNKNSFFNGINVESKHHLANPEIKTFAHNSYPILNQGLINCNPLQCLGYDSNQRNKHNVVYIKKNEYLNKNIGSIINVLKREGLRKISTHNGKFESFSNMDNKNVYMEGLNIQ

SEQ ID NO: 27 - DNA, 714 bp, Artificial Sequence
  PfEMMA1 Frag 1
gacaatgtcaataacaataacaacaaagagtcctgtgacaacatcaagcacatgcgcacgaaaagcctgaactttgtgagccgtgagagctatggtgagcataagagcctggacgtctaccaggaatgctacgtgaaaaacaataagctgattaacaaagttaacgataagaaatacgaagataacaacaattcctatttgaacgaagatgacaatgcgtcgatgcagttctatgaagagactaacagcaatccgtacatcgttgaccaagagaacaatatgaaaaactacgtgaataacgtgctgtacaataacaattctaattactacgttgattccaagaattatgacaaaagcaaagaaaatgccgagaacaagagcgacgacatcctgaataacgaaaacattcacaccctgaaagatcagaagaaaaagatccagaacaataacgaattcattagcgagcaagcggacatcgagaatatccgtaatagccaagaagaggtttatgagaaagaacatgagccgctgtgggtgattaatgctagcaacgaagagaagaaaagctacgaagaattgatttacagcgatatgagcagcaaccgcgtcacgaagaataagtatagcgatatgaataacgttgaagtcctgctgaatgaggacaatctgctgaccaccgagaagtataaagttcagctggagaaagaaaacaag

SEQ ID NO: 28 - DNA, 252 bp, Artificial Sequence
  pFEMMA1 Cterm
ctgaagattaacaaaaaagttccgaagtataaactggagaacatgaatatcaatagcccgaacattccgtatacctacttgtttatctgtgacggttccaattatctgtgcatcaacgacaacagcctgaataacgaagtgtatgagaacaagatgaagctgaacaatatcattggctactaccattacattaacctgaatcgcctgacgtactatctggaaaaagttaacgccaatttcgtctacaatcac

SEQ ID NO: 29 - DNA, 675 bp, Artificial Sequence
  PbEMMA1 frag1
accaatagctatacgaacaacatgctgattaatgattgtaacaatctgcagaacaagagccatcgctatatccgtgcgaagtccttgaatgcgctgcgcgacagcattctgcacagcaaatctttaaaggtctacgaagattgctgcatcaagaacaaaaatgaaaacatcacgaatcacgactacaatatgaaagacatctacaagcgttgcagctacatcggtaatgaaaacacccatgaaatcttctgcaccgagaaatctgagatctgtgagaacgatctgaacggttataacattaaaccggtgctgattaagtcccacgccgatgaaacgaatgaaaagaccaataacattctgctgaatgacatcaacagccagattgcacacaagaaaaatgacaataacgaaaacatttacttgctggagaaagagaacaagattgagcaaatcctgaccaacctgtccaataatgaaaacgatagcgacaattgtaccagcgtgacgattcagcactacaccgttaacaatgccgacaacaataaagtcggcgatcacgttctgtcgagctacaacatttgcgagcaagaagagagcctgtggatgccgaatcagtataagatcgagaaagcgatcctgaaagagagcagcgagaactatattcataaagaggaa

SEQ ID NO: 30 - DNA, 699 bp, Artificial Sequence
  PbEMMA1 frag2
ccgaaccagtataagattgagaaagcaatcctgaaagagagctccgagaattacattcataaggaagaattccagaacggtctggtgaagtgccgtaacaacttcatcgataatggtatcaaccaatgcgaagatatgctgagcggcgttattgagaaaaaagaaaataccaattgtaagattaatgacgacgagaaaaagaatttcaacgataaagttgaccagaatgataaagaaatgattgacatctgcgtgaacagctctagcgagaactgctacatcaagaataacagcaacaatcacaacaacaagtatagcattgaaaaagaatccaagaatctgaaagatgtcgcccacgacgactttgagcacaccacgatgaccacctttacgattatcaacccgaacatcattaccagctctaccttcgagaataatgcatcgacgtgtgagatgattaagaactacaatccgaataaagagtgccgcaacgtcgagatcaataacgacgcgaaaaaagaaaaagaaaaatacgacaataagaaaaagatcactaagagcaccaataacagcagcaagatcggcaaaaacaataagctgtgtaagctgaaaaaagcgagcattacgaagaaaaactggaagaaccgtgagattggtgagctgagcgagaatgaaatctacaagctgccgagctatttg

(19) United States
(12) Patent Application Publication
(10) Pub. No.: US 2022/0072115 A1
(43) Pub. Date: Mar. 10, 2022

(54) MALARIA ANTIGENS ON THE SURFACE OF ERYTHROCYTES AND MEROZOITES AND PROTECTIVE ANTIBODIES

(71) Applicant: Rhode Island Hospital, Providence, RI (US)

(72) Inventors: Ian Charles Michelow, Needham, MA (US); Jonathan Kurtis, Providence, RI (US)

(21) Appl. No.: 17/289,133

(22) PCT Filed: Nov. 4, 2019
(86) PCT No.: PCT/US2019/059671

§ 371 Date: Apr. 27, 2021

Related U.S. Application Data
(60) Provisional application No. 62/757,151, filed on Nov. 7, 2018.

(51) Int. Cl. A61K 39/015; C07K 16/20; A61P 37/04; A61P 33/06

(52) U.S. Cl. CPC A61K 39/015; C07K 16/205; A61K 2039/545; A61P 33/06; A61P 37/04

(57) ABSTRACT

Provided herein are compositions and methods for treating and preventing malaria. The vaccine of the invention is directed to the protein encoded by the PF3D7_1134300 gene, or fragments of the protein, which are Pf Erythrocyte Membrane and Merozoite Antigenl (PfEMMAI). Exemplary fragments include Fragment 1, Fragment 2, or a C-terminal fragment of PfEMMAI.

### RELATED APPLICATIONS

This application claims the benefit of priority under 35 U.S.C. § 119(e) to U.S. Provisional Application No. 62/757,151, filed Nov. 7, 2018, the entire contents of which is incorporated herein by reference in its entirety.

### GOVERNMENT SUPPORT

This invention was made with government support under 1P20GM104317 awarded by National Institutes of Health. The government has certain rights in the invention.

### INCORPORATION BY REFERENCE OF SEQUENCE LISTING

The contents of the sequence listing text file named “21486-639001WO_Sequence_Listing_ST25.txt”, which was created on Oct. 31, 2019 and is 57,344 bytes in size, is hereby incorporated by reference in its entirety.

### BACKGROUND

Plasmodium falciparum (Pf) malaria claims more than 440,000 lives each year despite decades of intensive public health interventions in endemic areas (World Health Organization (WHO), Geneva, Switzerland, 2017, vol. 12/15/17). Global efforts to combat malaria have met with rapidly emerging resistance to front-line antimalarial agents and insecticides (J. Hemingway et al., PLoS Biol 14, e1002380 (2016)). In addition, RTS,S, the most advanced malaria vaccine candidate had limited efficacy and durability in phase III trials (A. Olotu et al., N. Engl. J. Med 374, 2519-2529 (2016)). At least 50 predominantly subunit malaria vaccines as well as whole sporozoite vaccines are currently under investigation in pre-clinical or clinical settings. However, the subunit candidates are derived from fewer than 25 unique antigens (WHO, Geneva, Switzerland (2017).

Accordingly, there is an urgent need to discover novel targets to expand the limited repertoire of immune-based interventions.

### SUMMARY OF THE INVENTION

The vaccine of the invention is directed to the protein encoded by the PF3D7_1134300 gene, or fragments of the protein, which are Pf Erythrocyte Membrane and Merozoite Antigen1 (PfEMMA1). Exemplary fragments include Fragment 1, Fragment 2, or a C-terminal fragment of PfEMMA1. This protein, PfEMMA1, is expressed in blood stage malaria parasites and is dually localized to the red blood cell (RBC) and merozoite surfaces. The vaccine based on PfEMMA1 or fragments thereof, leads to a double protection by anti-PfEMMA1 antibodies, by blocking the merozoite invasion of red blood cells (RBCs) as well as disrupting interactions between RBCs and host cells, for example by reducing cytoadherence, rosette formation of erythrocytes, and/or deleterious cytokine production by innate immune cells. The PfEMMA1 protein is highly conserved in several species of Plasmodium parasites, including human, non-human primate, rodent and avian strains. PfEMMA1 is uniquely differentiated from several other surface exposed proteins, including the variant surface antigens (VSA), because it is encoded by a single copy gene with low polymorphism and has been characterized as a target of antibodies in plasma from resistant, but not susceptible Tanzanian children.

The vaccine is useful for blocking the merozoite invasion of red blood cells (RBCs) as well as disrupting interactions between RBCs and host cells, for example by reducing cytoadherence, rosette formation of erythrocytes, and/or deleterious cytokine production by innate immune cells. In addition, the vaccine reduces or inhibits transmission of parasite sexual forms from humans to mosquitoes i.e., the vaccine has a transmission blocking function. The vaccine achieves this protection by directly blocking or inhibiting gametocytes in the human circulation or by inhibiting the formation of oocysts in the mosquito midgut after the mosquito ingests gametocytes, thereby preventing replication of the parasite sexual forms.

For example, the invention features a composition that comprises, consists essentially of, or consists of a purified polypeptide comprising the amino acid sequence of SEQ ID NO: 1, 3, 5, 7, 8, 9, 10, 11, 12, 13, 14, 15, 16, 17, 18, 19, 20, 21, 23, 24, 25, or 26 or a purified nucleic acid encoding a gene product that comprises the amino acid sequence of SEQ ID NO: 1, 3, 5, 7, 8, 9, 10, 11, 12, 13, 14, 15, 16, 17, 18, 19, 20, 21, 23, 24, 25, or 26, along with a pharmaceutically-acceptable excipient and/or an adjuvant. An immune response elicited by immunization with these vaccine antigens inhibits parasite invasion of RBCs and/or parasite growth in RBCs. For example, the composition comprises a purified antigen that elicits an anti-PfEMMA1 antibody response. Alternatively, a passive immunization approach is used. In the latter case, the composition comprises a purified antibody that specifically binds to one or more of the parasite antigens that are involved in invasion of RBCs and/or parasite growth in RBCs. For example, the composition comprises an anti-PfEMMA1 antibody or antigen binding fragment thereof. Thus, a method for preventing or reducing the severity of malaria is carried out by administering to a subject a composition that inhibits parasite invasion of RBCs and/or parasite growth in RBCs.

The invention also includes a vaccine for preventing or reducing the severity of malaria comprising a polypeptide composition, wherein the polypeptide comprising an amino acid sequence selected from the group consisting of SEQ ID NOs: 1, 3, 5, 7, 8, 9, 10, 11, 12, 13, 14, 15, 16, 17, 18, 19, 20, 21, 23, 24, 25, and 26 (antigenic polypeptides or protein fragments). A vaccine for preventing or reducing the severity of malaria comprising a composition comprising a purified polypeptide comprising an amino acid sequences of SEQ ID NO: 1, 3, 5, 7, 8, 9, 10, 11, 12, 13, 14, 15, 16, 17, 18, 19, 20, 21, 23, 24, 25, 26 or an antigenic fragment thereof. Also included is a vaccine for preventing or reducing the severity of malaria comprising a composition that includes a purified polypeptide consisting essentially of an amino acid sequence of SEQ ID NO: 1, 3, 5, 7, 8, 9, 10, 11, 12, 13, 14, 15, 16, 17, 18, 19, 20, 21, 23, 24, 25, 26 or an antigenic fragment thereof. For example, the antigenic fragment comprises an amino acid sequence of SEQ ID NO: 7, 8, 9, 10, 11, 12, 13, 14, 15, 16, 17, 18, 19 or 20. Further included is a vaccine for preventing or reducing the severity of malaria comprising a composition that includes a polynucleotide comprising a nucleic acid sequence of SEQ ID NO: 2, 4, 6 or a fragment thereof.

The invention also features an isolated peptide having at least 90%, 95% or 99% identity with the sequence of SEQ ID NO: 1, 3 or 5; a peptide encoded by a nucleic acid sequence having at least 90%, 95% or 99% identity with the sequence of SEQ ID NO: 2, 4 or 6, in a vaccine composition for treatment or prevention of P. falciparum malaria.

The invention also features an isolated nucleic acid sequence comprising a nucleic acid sequence having at least 90%, 95% or 99% identity with the sequence of SEQ ID NO: 2, 4, 6, or any fragment thereof in a vaccine composition for treatment or prevention of P. falciparum malaria.

Antigens for use in a malaria vaccine include one or more of the following polypeptides (or fragments thereof) that elicit a clinically relevant decrease in the severity of the disease or that reduce/prevent infection or spread of parasites, and/or reduce or inhibit transmission of parasite sexual forms from humans to mosquitoes i.e., the vaccine has a transmission blocking function, which refers to protection achieved by directly blocking or inhibiting gametocytes in the human circulation or by inhibiting the formation of oocysts in the mosquito midgut after the mosquito ingests gametocytes, thereby preventing replication of the parasite sexual forms. The polypeptides/fragments that elicit a parasite-specific antibody and/or cellular immune response include those encoded by the following nucleotides: SEQ ID NO: 1, 3, 5, 7, 8, 9, 10, 11, 12, 13, 14, 15, 16, 17, 18, 19, 20, 21, 23, 24, 25, and/or 26.

Also provided herein is a vector or a host cell expressing one or more isolated peptides or one or more isolated nucleic acid sequences described herewith.

Another aspect of the invention relates to vaccine composition. The vaccine composition contains one or more isolated peptides or one or more isolated nucleic acid sequences described herewith. The peptide vaccine may also contain an adjuvant. Exemplary adjuvants include aluminum salts, such as aluminum phosphate and aluminum hydroxide. Another exemplary adjuvant is an oil adjuvant such as the Montanide ISA series, e.g., ISA 50 V2 or ISA 720 VG. The deoxyribonucleic acid (DNA) vaccine contains a eukaryotic vector to direct/control expression of the antigen in the subject to be treated.

The vaccine of the invention provides a new regimen in treating or preventing P. falciparum malaria in a subject. Accordingly, the invention described herein further provides a method of treating or preventing P. falciparum malaria in a subject in need by administering the vaccine to the subject. In embodiments, the subject is an infant from 6 weeks of age and older. In embodiments, the subject includes children of all ages. In embodiments, the subject is an adult. Preferably, the subject is a child under 5 years of age. More preferably, the subject is at least about 6-8 weeks of age. The vaccine is also suitable for administration to older children or adults. The vaccine is also suitable for administration to pregnant females. The vaccine can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form. Preferably, the vaccine is administered intramuscularly. The dosing regimen that can be used in the methods of the invention includes, but is not limited to, daily, three times weekly (intermittent), two times weekly, weekly, or every 14 days. Alternatively, dosing regimen includes, but is not limited to, monthly dosing or dosing every 6-8 weeks. The vaccine described herein can be administered intramuscularly once every two weeks for 1, 2, 3, 4, or more times alone or in combination with 1, 2, 3, 4, or more additional vaccines in a subject, preferably a human subject. In embodiments, the vaccine described herein can be administered intramuscularly once every 6-8 weeks and three times in total, over a period up to 24 weeks. In embodiments, the vaccine described herein can be administered intramuscularly once at 6, 10 and 14 weeks of age then optionally a booster between 12-24 months of age and subsequently annually if needed. In embodiments, the vaccine described herein can be administered intramuscularly once at 2, 4, and 6 months of age and optionally a booster at 12-24 months. In embodiments, the vaccine described herein can be administered intramuscularly once at 5, 7, and 9 months of age and optionally a booster at 12-24 month. In embodiments, the vaccine described herein can be administered intramuscularly once at 6, 8, 10 months of age and optionally a booster at 12-24 months. One exemplary additional vaccine contains an inhibitor of parasite liver invasion, such as RTS,S (Mosquirix). Another exemplary additional vaccine contains an inhibitor of parasite RBC invasion, such as MSP-1. The vaccine can be made by any known method in the art.

Also provided herein are antibodies that specifically bind to an antigen comprising the isolated peptides described herein, i.e. Frag 1, Frag 2 and C-term, and a method of treating P. falciparum malaria in a subject in need of by administering a therapeutically effective amount of such antibody to the subject. The P. falciparum malaria can be acute P. falciparum malaria.

Also provided herein is a method of treating P. falciparum malaria in a subject in need of by administering a therapeutically effective amount of an antibody described herewith to the subject. Preferably, the antibody is a purified monoclonal antibody, e.g., one that has been raised to and is specific for the protein of SEQ ID NO: 1, 3, 5, 7, 8, 9, 10, 11, 12, 13, 14, 15, 16, 17, 18, 19, 20, 21, 23, 24, 25, or 26. For example, the monoclonal antibody is a humanized antibody. The treatment can be initiated at an early stage after the appearance of recrudescent parasites. The symptoms of the subject may be mild or absent and parasitemia is low but increasing, for example from range 4,000-10,000/ul. Alternative, the subject may have fever <38.5° C. without any other accompanying symptom. The subject can be a child under 10 years of age. The subject can also be an elder child or an adult. In one example, the subject is characterized as suffering from acute P. falciparum malaria but has not responded to treatment with anti-malarial drugs. In this passive immunity approach, the purified humanized monoclonal antibody that binds specifically to the protein of SEQ ID NO: 1, 3, 5, 21, 7, 8, 9, 10, 11, 12, 13, 14, 15, 16, 17, 18, 19, 20, 21, 23, 24, 25, or 26 is administered to the subject to kill the infective agent and/or inhibit RBC invasion. In embodiments, the antibodies described herein bind to any antigenic fragment of the protein of SEQ ID NO: 1, 3, 5, 21, 23, 24, 25, or 26. For example, the antibodies describe herein bind to an antigen having SEQ ID NO: 7, 8, 9, 10, 11, 12, 13, 14, 15, 16, 17, 18, 19 or 20.

The antibody can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form. Preferably, the antibody is administered intravenously or intramuscularly. For example, the antibody is administered in 1-2 gram amounts, 1, 2, 3, or 4 times. The dosing regimen that can be used in the methods of the invention includes, but is not limited to, daily, three times weekly (intermittent), two times weekly, weekly, or every 14 days. Alternatively, dosing regimen includes, but is not limited to, monthly dosing or dosing every 6-8 weeks. The antibody of the present invention can be administered intravenously once, twice or three times alone or in combination with 1, 2, 3, 4, or more additional therapeutic agents in a subject, preferably a human subject. The additional therapeutic agent is, for example, one, two, three, four, or more additional vaccines or antibodies, an antimalarial artemisinin-combination therapy, or an immunotherapy. Any suitable therapeutic treatment for malaria may be administered. The additional vaccine may comprise an inhibitor of parasite liver invasion or an inhibitor of parasite RBC invasion. Such additional vaccines include, but are not limited to, anti-RBC invasion vaccines (MSP-1 or PfRH5), RTS,S (Mosquirix), NYVAC-Pf7, CSP, and [NANP]19-5.1. The antibody of the invention can be administered prior to, concurrently, or after other therapeutic agents.

In some embodiments, the PfEMMA1 antigen(s) described herein is combined with other malarial antigens. In other embodiments, a vaccine comprises PfEMMA1 antigen fragments but does not comprise a full-length PfEMMA1 protein. In yet other embodiments, a vaccine formulation comprises PfEMMA1 antigens or PfEMMA1 antigen fragments and does not comprise other malarial antigens such as PfSEA antigens or PfGARP antigens, e.g., as described in U.S. Pat. No. 9,662,379, hereby incorporated by reference. Alternatively, PfEMMA1 antigen fragments may be combined with other malarial antigens in a combination vaccine formulation.

Amounts effective for this use will depend on, e.g., the antibody composition, the manner of administration, the stage and severity of malaria being treated, the weight and general state of health of the patient, and the judgment of the prescribing physician, but generally range for the treatment from about 10 mg/kg (weight of a subject) to 300 mg/kg, preferably 20 mg/kg-200 mg/kg.

The invention described herein further provides a kit for determining the presence of antibody to P. falciparum, P. vivax, P. ovale, P. malariae and/or P. knowlesi in a sample obtained from a subject. A “sample” is any bodily fluid or tissue sample obtained from a subject, including, but is not limited to, blood, serum, urine, and saliva. The kit contains an antigen or an antibody of the present invention and optionally one or more reagents for detection.

The kit may also contain a sample collection means, storage means for storing the collected sample, and for shipment. The kit further comprises instructions for use or a CD, or CD-ROM with instructions on how to collect sample, ship sample, and means to interpret test results. The kit may also contain an instruction for use to diagnose malaria or a receptacle for receiving subject derived bodily fluid or tissue.

The kit may also contain a control sample either positive or negative or a standard and/or an algorithmic device for assessing the results and additional reagents and components. The kit may further comprise one or more additional compounds to generate a detectable product.

An antibody described herein may be a polyclonal antisera or monoclonal antibody. The term antibody may include any of the various classes or sub-classes of immunoglobulin (e.g., IgG, IgA, IgM, IgD, or IgE derived from any animal, e.g., any of the animals conventionally used, e.g., sheep, rabbits, goats, or mice).

The terms “monoclonal antibody” or “monoclonal antibody composition” as used herein refer to a preparation of antibody molecules of single molecular composition. A monoclonal antibody composition displays a single binding specificity and affinity for a particular epitope.

An “antibody fragment” comprises a portion of an intact antibody, preferably the antigen binding and/or the variable region of the intact antibody. Non-limiting examples of antibody fragments include Fab, Fab*, F(ab′)₂ and Fv fragments; diabodies; linear antibodies; single-chain antibody molecules and multispecific antibodies formed from antibody fragments.

By “antigen” is defined as a molecule that provokes an immune response. This immune response may involve either antibody production, or the activation of specific immunologically-competent cells, or both. For example, any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein.

A “vaccine” is to be understood as meaning a composition for generating immunity for the prophylaxis and/or treatment of diseases. Accordingly, vaccines are medicaments which comprise antigens and are intended to be used in humans or animals for generating specific defense and protective substance by vaccination.

“Patient” or “subject in need thereof” or “subject” refers to a living member of the animal kingdom suffering from or who may suffer from the indicated disorder. In embodiments, the subject is a member of a species comprising individuals who may naturally suffer from the disease. In embodiments, the subject is a mammal. Non-limiting examples of mammals include rodents (e.g., mice and rats), primates (e.g., lemurs, bushbabies, monkeys, apes, and humans), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines, donkeys, mules, bison, goats, camels, and sheep), and deer. A “subject” in the context of the invention described herein is preferably a mammal. The mammal can be a human, non-human primate, mouse, rat, dog, cat, horse, or cow, but are not limited to these examples. A subject can be male or female. A subject can be a child or an adult. A subject can be a pregnant female. A subject can be one who has been previously diagnosed or identified as having malaria, and optionally has already undergone, or is undergoing, a therapeutic intervention for the malaria. Alternatively, a subject can also be one who has not been previously diagnosed as having malaria, but who is at risk of developing such condition, e.g. due to infection or due to travel within a region in which malaria is prevalent. For example, a subject can be one who exhibits one or more symptoms for malaria.

A subject “at risk of developing malaria” in the context of the invention described herein refers to a subject who is living in an area where malaria is prevalent, such as the tropics and subtropics areas, or a subject who is traveling in such an area. Alternatively, a subject at risk of developing malaria can also refer to a subject who lives with or lives close by a subject diagnosed or identified as having malaria.

The terms “subject,” “patient,” “individual,” etc. are not intended to be limiting and can be generally interchanged. That is, an individual described as a “patient” does not necessarily have a given disease, but may be merely seeking medical advice.

As used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) or polypeptide is free of the amino acid sequences or nucleic acid sequences that flank it in its naturally-occurring state. Purified also defines a degree of sterility that is safe for administration to a human subject, e.g., lacking infectious or toxic agents.

Purity is measured by any appropriate standard method, for example, by column chromatography, thin layer chromatography, or high-performance liquid chromatography (HPLC) analysis. The nucleotides and polypeptides are purified and used in a number of products for consumption by humans as well as animals, such as companion animals (dogs, cats) as well as livestock (bovine, equine, ovine, caprine, or porcine animals, as well as poultry). A purified or isolated polynucleotide (ribonucleic acid (RNA) or DNA) is free of the genes or sequences that flank it in its naturally occurring state. For example, the DNA is a complementary DNA (cDNA). “Purified” also defines a degree of sterility that is safe for administration to a human subject, e.g., lacking infectious or toxic agents.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally occurring organic molecules with they are naturally associated. A purified or isolated polynucleotide (ribonucleic acid (RNA) or deoxyribonucleic acid (DNA)) or polypeptide is free of the amino acid sequences or nucleic acid sequences that flank it in its naturally-occurring state. Purified also defines a degree of sterility that is safe for administration to a human subject, e.g., lacking infectious or toxic agents.

By the terms “effective amount,” “effective dose,” and “therapeutically effective amount” of a formulation or formulation component is meant a sufficient amount of the formulation or component to provide the desired effect. For example, “an effective amount” of a vaccine is an amount of a compound required to reduce or inhibit human to mosquito transmission (i.e. transmission blocking, which refers to protection achieved by directly blocking or inhibiting gametocytes in the human circulation or by inhibiting the formation of oocysts in the mosquito midgut after the mosquito ingests gametocytes, thereby preventing replication of the parasite sexual forms), inhibit growth of parasites in RBCs or elicit an antibody or cellular immune response to the vaccine antigen(s). Ultimately, the attending physician or veterinarian decides the appropriate amount and dosage regimen.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean inhibiting the progression of the condition, disorder or disease, slowing the progression of the condition, disorder or disease temporarily, although in some instances, it involves halting the progression of the condition, disorder or disease. The terms “preventing” and “prevention” refer to the administration of an agent or composition to a clinically asymptomatic individual who is susceptible or predisposed to a particular adverse condition, disorder, or disease, and thus relates to the prevention of the occurrence of symptoms and/or their underlying cause.

As used herein, the terms “treat” and “prevent” are not intended to be absolute terms. Treatment can refer to a 10%, 20%, 30%, 40%, 50%, 60%, 70%, 80%, 90%, or 100% reduction in the severity of an established disease, condition, or symptom of the disease or condition. A method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. References to decreasing, reducing, or inhibiting include a change of 10%, 20%, 30%, 40%, 50%, 60%, 70%, 80%, 90% or greater as compared to a control level and such terms can include but do not necessarily include complete elimination. The severity of disease is reduced by at least 10%, as compared, e.g., to the individual before administration or to a control individual not undergoing treatment. In some aspects, the severity of disease is reduced by at least 25%, 50%, 75%, 80%, or 90%, or in some cases, no longer detectable using standard diagnostic techniques.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps and permits those that do not materially affect the basic and the characteristic(s) of the claimed invention.

As used herein, the term “about” in the context of a numerical value or range means±10% of the numerical value or range recited or claimed, unless the context requires a more limited range.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg up to and including 5.0 mg.

As used in the description herein and throughout the claims that follow, the meaning of “a,” “an,” and “the” includes plural reference unless the context clearly dictates otherwise.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, Genbank/NCBI accession numbers, and other references mentioned herein are incorporated by reference in their entirety. In the case of conflict, the present specification, including definitions, will control. In addition, the materials, methods, and examples are illustrative only and not intended to be limiting.

### BRIEF DESCRIPTION OF THE DRAWINGS

FIG. 1A is a schematic of the predicted protein structure. There is a putative transmembrane helical peptide at aa 2089-2135 (red) near the C-terminus as predicted by the Argos transmembrane analytic method (Mac Vector, Inc.). Identified Domain refers to the full sequence identified by our phage library screen; fragments (frag) 1 and 2 refer to overlapping recombinant polypeptides that were expressed in E. coli.

FIG. 1B is a graph showing that nonsynonymous diversity of PfEMMA1 was low in 209 parasites collected from two African countries compared with other known vaccine candidates.

FIG. 1C is a graph showing that the Tajima's D statistic is negative, indicating that variants in the gene are primarily at very low frequencies. Dashed lines indicate 2.5% and 97.5% distribution cutoffs.

FIG. 1D is an image of a protein gel and immunoblot showing the expression and purification of rPfEMMA1 fragment 1 from E. coli inclusion bodies. Theoretically predicted sizes of the fragment conjugated to an S Tag and 10× His tags is 31 kDa. Lane 1, urea-solubilized inclusion bodies; lane 2, nickel chelate chromatography of lane 1; lane 3, anion exchange chromatography of lane 2 Amino acid sequences of all bands in lane 3 of SDS-PAGE gels were verified by LC-MS/MS.

FIG. 1E is an image of a protein gel and immunoblot showing the expression and purification of rPfEMMA1 fragment 2 from E. coli inclusion bodies. Theoretically predicted sizes of the fragment conjugated to an S Tag and 10× His tags is 32 kDa. Lane 1, urea-solubilized inclusion bodies; lane 2, nickel chelate chromatography of lane 1; lane 3, anion exchange chromatography of lane 2 Amino acid sequences of all bands in lane 3 of SDS-PAGE gels were verified by LC-MS/MS.

FIG. 1F is an image of an immunoblot of Pf3D7-infected erythrocyte lysates. Uninfected (uninf) human RBCs or RBCs infected (inf) with mixed trophozoite- and schizont-stage parasites were resolved with a 4-15% polyacrylamide gel and probed with murine pre-immune sera or anti-PfEMMA1 fragment 2 antisera. Arrows indicate a double band (>250 kDa).

FIG. 2 is a series of images of immunofluorescence confocal microscopy showing structural and temporal localization of PfEMMA1. Permeabilized P. falciparum 3D7 infected RBCs were probed with mouse anti-PfEMMA1 fragment 2 (green) and rabbit anti-PfAMA1 (red), and counterstained with 4′,6′-diamino-2-phenylindole (DAPI) to label parasite nuclei. PfEMMA1 was detected after the ring stage when it was expressed in a stippled pattern outside the parasite and then aggregates at the RBC periphery but does not colocalize with PfAMA1, a merozoite marker. Scale bar, 5 μm. DIC, digital interference contrast microscopy.

FIG. 3A is an image of immunofluorescence confocal microscopy showing permeabilized P. falciparum 3D7 infected RBCs probed with mouse anti-PfEMMA1 fragment 2 (green) and rabbit antibodies against PfREX1, a structural protein of Maurer's clefts (red), and counterstained with 4′,6′-diamino-2-phenylindole (DAPI) to label parasite nuclei. PfEMMA1 colocalized with PfREX1.

FIG. 3B is an image of immunofluorescence confocal microscopy showing PfEMMA1 colocalized with PfSBP1, a structural protein of Maurer's clefts, in permeabilized P. falciparum 3D7 infected RBCs.

FIG. 3C is an image of immunofluorescence confocal microscopy showing that PfEMMA1 is expressed at the periphery of permeabilized infected RBCs in close association with glycophorin A (GPA), a host cell surface glycoprotein.

FIG. 3D is an immunofluorescence confocal microscopy image showing that in non-permeabilized parasite-infected RBCs (pRBCs), anti-PfEMMA1 fragment 2 antibodies specifically labeled proteins on the exofacial surface of pRBCs; the control antibody, rabbit anti-MSP4 antibodies did not penetrate the RBC surface membrane.

FIG. 3E is an immunofluorescence confocal microscopy image showing that anti-PfEMMA1 fragment 2 antibodies labeled proteins in residual RBC membranes of remnant ghost cells after parasite egress.

FIG. 3F is a series of images showing exofacial surface labeling of live non-permeabilized RBCs infected with late-stage parasites (Hoechst and mitotracker double positive cells). RBCs were labeled with mouse anti-PfEMMA1 fragment 1 primary antibodies and anti-mouse IgG1-FITC secondary antibodies and showed expression in fewer than 5% of cells counted. pRBCs that were predigested with trypsin or chymotrypsin, had >4.5-fold reduction (approximately 80%) in PfEMMA1 surface expression. Scale bar, 5 μm. DIC, digital interference contrast microscopy. FSC, forward scatter.

FIG. 3G is a series of images showing exofacial surface labeling of live non-permeabilized RBCs infected with late-stage parasites (Hoechst and mitotracker double positive cells). RBCs were labeled with mouse anti-PfEMMA1 fragment 2 primary antibodies and anti-mouse IgG1-FITC secondary antibodies that showed expression in fewer than 5% of cells counted. pRBCs that were predigested with trypsin or chymotrypsin, had >4.5-fold reduction (approximately 80%) in PfEMMA1 surface expression. Scale bar, 5 μm. DIC, digital interference contrast microscopy. FSC, forward scatter.

FIG. 4A is a low power immunogold electron micrograph showing that in live non-permeabilized pRBCs, PfEMMA1 localized to the exofacial surface of the RBC membrane in close proximity to electrodense knobs.

FIG. 4B is a high power immunogold electron micrograph showing that in live non-permeabilized pRBCs, PfEMMA1 localized to the exofacial surface of the RBC membrane in close proximity to electrodense knobs. Late-stage infected RBCs were probed with mouse anti-PfEMMA1 fragment 2 antibodies (6 nm gold particles; black arrows) and rabbit anti-glycophorin C (10 nm gold particles; white arrows with black outline).

FIG. 4C is a low power immunogold electron micrograph showing that in permeabilized pRBCs, PfEMMA1 localized to Maurer's cleft as well as the inner leaflet of the RBC membrane.

FIG. 4D is a high power immunogold electron micrograph showing that in permeabilized pRBCs, PfEMMA1 localized to Maurer's cleft as well as the inner leaflet of the RBC membrane. Late-stage infected RBCs were probed with mouse anti-PfEMMA1 fragment 2 antibodies (6 nm gold particles; black arrows) and rabbit anti-glycophorin C (10 nm gold particles; white arrows with black outline).

FIG. 5A is a series of immunofluorescence confocal microscopy images showing a non-permeabilized Plasmodium falciparum 3D7 merozoite which was probed with antibodies against PfEMMA1 fragment 2 and PfRH5. It was shown that the portion of PfEMMA1 labeled by anti-Frag 2 antibodies is displayed on the extrafacial surface of merozoites in close association with PfRH5.

FIG. 5B is a series of immunofluorescence confocal microscopy images showing a non-permeabilized Plasmodium falciparum 3D7 merozoite which was probed with antibodies against PfEMMA1 fragment 2 and PfAMA1. It was shown that the portion of PfEMMA1 labeled by anti-Frag 2 antibodies is displayed on the extrafacial surface of merozoites but did not colocalize with PfAMA1.

FIG. 5C is a series of immunofluorescence confocal microscopy images showing a non-permeabilized Plasmodium falciparum 3D7 merozoite which was probed with antibodies against PfEMMA1 fragment 2 and PfMSP1. It was shown that the portion of PfEMMA1 labeled by anti-Frag 2 antibodies is displayed on the extrafacial surface of merozoites but did not colocalize with PfMSP1.

FIG. 5D is a series of immunofluorescence confocal microscopy images showing contact between an uninfected RBC and the apex of Plasmodium falciparum 3D7 merozoite where PfEMMA1 is highly expressed. Both the RBC and merozoite were permeabilized and probed with antibodies against PfEMMA1 and PfRH5, which were in close proximity.

FIG. 5E is a series of immunofluorescence confocal microscopy images showing a non-permeabilized Plasmodium falciparum 3D7 schizont that was probed with antibodies against PfEMMA1 C-terminus and glycophorin C (GPC). It was shown that the PfEMMA1 C-terminus is not displayed on the extrafacial surface of non-permeabilized pRBCs.

FIG. 5F is a series of immunofluorescence confocal microscopy images showing a non-permeabilized Plasmodium falciparum 3D7 merozoite that was probed with antibodies against PfEMMA1 C-terminus and glycophorin C (GPC). It was shown that the PfEMMA1 C-terminus is not displayed on the extrafacial surface of non-permeabilized merozoites.

FIG. 5G is a series of immunofluorescence confocal microscopy images showing a permeabilized Plasmodium falciparum 3D7 trophozoite that was probed with antibodies against PfEMMA1 C-terminus and PfMSP4. It was shown that full length PfEMMA1 containing the C-terminus is retained within the developing trophozoite i.e. it is not exported into the RBC cytoplasm.

FIG. 5H is a series of immunofluorescence confocal microscopy images showing a permeabilized Plasmodium falciparum 3D7 merozoite which was probed with antibodies against PfEMMA1 C-terminus and PfMSP4. It was shown that full length PfEMMA1 containing the C-terminus is expressed in merozoites. For FIG. 5A-H, counterstains included DAPI, Plasmodium falciparum reticulocyte-binding protein homologue 5 (PfRH5), Plasmodium falciparum apical membrane antigen 1 (PfAMA1), and Plasmodium falciparum merozoite surface proteins (PfMSP) 1 and 4. Scale bar for RBCs, 5 μm and merozoites, 2 μm. DIC, digital interference contrast microscopy.

FIG. 6A is a bar graph showing results of a growth/invasion inhibition assay (GIA) in which Plasmodium falciparum 3D7 parasites were inhibited up to 68% by purified polyclonal immunoglobulins against recombinant PfEMMA1 Fragments 1 and 2 in a dose-dependent manner. Inhibitory concentration (IC)₅₀ of these antibodies are shown.

FIG. 6B is a bar graph showing results of a growth/invasion inhibition assay (GIA) in which Plasmodium falciparum Dd2 parasites were significantly inhibited by purified polyclonal immunoglobulins against recombinant PfEMMA1 Fragments 1 and 2 in a dose-dependent manner.

FIG. 6C is a bar graph showing results of a growth/invasion inhibition assay (GIA) in which the neutralizing effect of PfEMMA1 immunoglobulins on Plasmodium falciparum 3D7 parasites was reversed by preincubating antibodies (anti-fragment 1 [αfrag1] and 2 [αfrag2]; 2.5 mg/mL each) with recombinant proteins (recombinant fragment 1 [rfrag1] and 2 [rfrag2], respectively; 650 nM each). * P<0.003. PI, preimmune sera.

FIG. 6D is a graph showing that specific affinity-purified anti-PfEMMA1 fragments 1 and 2 immunoglobulins (0.02 and 0.06 mg/mL) from the plasma of immune Kenyan adults significantly inhibited Plasmodium falciparum 3D7 parasites in growth/invasion inhibition assays (GIA). Experiments were performed in triplicate and are representative of 3 independent assays. * P<0.004. PI, preimmune sera. NA ctrl, North American control.

FIG. 7A is a Kaplan-Meier survival curve showing that mice immunized with rPbEMMA1 survived longer than adjuvant controls after challenge with P. berghei ANKA. In Experiment 1: BALB/cJ mice were immunized three times via intraperitoneal injection (i.p.) with rPbEMMA1 fragment 1 (n=5) and survived significantly longer than mice immunized with adjuvant alone (n=5) after i.p. challenge with 10⁴ P. berghei ANKA-infected RBCs.

FIG. 7B is a Kaplan-Meier survival curve showing that mice immunized with rPbEMMA1 survived longer than adjuvant controls after challenge with P. berghei ANKA. In Experiment 2: BALB/cJ mice were immunized four times by subcutaneous injection (s.c.) with rPbEMMA1. Two of five mice (40%) immunized with fragment 1 eradicated parasitemia after i.p. challenge with 10⁴ P. berghei ANKA-infected RBCs and one of these two mice survived a second challenge (indicated by arrow) with the same inoculum of P. berghei ANKA.

FIG. 7C is a Kaplan-Meier survival curve showing that mice immunized with rPbEMMA1 survived longer than adjuvant controls after challenge with P. berghei ANKA. In Experiment 3: BALB/cJ mice were immunized four times s.c. with rPbEMMA1 fragment 1 (n=10), fragment 2 (n=10) or adjuvant controls (n=8). Mice immunized with fragment 1 survived i.p. challenge with 5×10⁴ P. berghei ANKA-infected RBCs significantly longer than mice immunized with rPbEMMA fragment 2 or adjuvant alone; one mouse from each group of actively immunized animals (10% each) eradicated parasitemia and survived a second challenge with 10⁴ P. berghei pRBC (indicated by arrow) significantly longer than two adjuvant control mice challenged concurrently.

FIG. 7D is a line graph showing P. berghei parasitemia from Experiment 1. Curves representing parasite densities for each mouse monitored longitudinally are shown. Mice that survived beyond the first seven days generally had high parasitemias (>10%). PbEMMA1 immunized mice survived longer than adjuvant controls despite high levels of circulating parasites.

FIG. 7E is a line graph showing P. berghei parasitemia from Experiment 2. Curves representing parasite densities for each mouse monitored longitudinally are shown. Mice that survived beyond the first seven days generally had high parasitemias (>10%). PbEMMA1 immunized mice survived longer than adjuvant controls despite high levels of circulating parasites.

FIG. 7F is a line graph showing P. berghei parasitemia from Experiment 3. Curves representing parasite densities for each mouse monitored longitudinally are shown. Mice that survived beyond the first seven days generally had high parasitemias (>10%). PbEMMA1 immunized mice survived longer than adjuvant controls despite high levels of circulating parasites.

FIG. 8A is a bar graph showing the frequency distribution curve of log-transformed naturally acquired antibody concentrations in plasma from Tanzanian children. Antibody concentrations were dichotomized at various percentiles for statistical analyses using generalized estimating equations to determine if there was an association with parasite density. There was a slight increase in numbers of children (dashed line) above the 97.5^{th} percentile.

FIG. 8B is a bar graph showing that high anti-PfEMMA1 fragment 1 antibodies (>97.5%) were associated with significantly lower parasite densities in Tanzanian children compared with lower antibody concentrations (67 vs. 124 parasites/200 WBC, respectively). * P=0.038.

FIG. 9 is a bar graph showing that purified immunoglobulins to PfEMMA1 recombinant proteins significantly inhibited parasite growth/invasion for PfW2 strain in a dose-dependent manner (* P<0.003). iRBC, infected red blood cells.

FIG. 10A is a schematic of the predicted protein structure of PbEMMA1 with a putative transmembrane helical peptide (red) near the C-terminus as predicted by the Argos transmembrane analytic method (MacVector, Inc.).

FIG. 10B is an image of a protein gel showing the purification of rPbEMMA1 fragment 1 from E. coli inclusion bodies which was achieved with sequential fast protein liquid chromatography (FPLC) using nickel chelate affinity chromatography and anion exchange chromatography. Proteins were resolved by SDS-PAGE and amino acid sequences were verified by LC-MS/MS.

FIG. 10C is an image of a protein gel showing the purification of rPbEMMA1 fragment 2 from E. coli inclusion bodies which was achieved with sequential fast protein liquid chromatography (FPLC) using nickel chelate affinity chromatography and anion exchange chromatography. Proteins were resolved by SDS-PAGE and amino acid sequences were verified by LC-MS/MS.

FIG. 11 is a bar graph showing the relationship between antibody levels to MSP1, MSP3, MSP7, LSA-N and LSA-C and resistance to parasitemia. IgG antibody levels were measured to MSP1 (19 kDa region of the 3D7 strain from BEI Resources/MR4), MSP3 (aa 99-265), MSP7 (aa 117-248), LSA-N(aa 28-150) and LSA-C (aa 1630-1909) using available plasma from 225 two-year old children enrolled in the birth cohort. Rate ratios (RR), [95% CIs] and P values for these proteins are: MSP1-19 (RR=2.04, [0.92-4.51], P=0.08), MSP3 (RR=0.38, [0.15-1.00], P=0.05), MSP7 (RR=0.63, [0.14-2.79], P=0.54), LSA-N(RR=2.36, [1.05-5.31], P=0.04), LSA-C(RR=2.36, [1.15-4.81], P=0.02).

FIG. 12 is a schematic model of PfEMMA1 processing and export mechanisms. After synthesis of full-length PfEMMA1 at the early trophozoite-stage, the protein is integrated in developing merozoites during schizogony and is detected within the parasite in infected RBCs as well as in merozoites after RBC egress by both anti-PfEMMA1 fragment 1 and 2, and anti-PfEMMA1 C-terminus antibodies. Alternatively, after synthesis in the endoplasmic reticulum, PfEMMA1 is trafficked to the parasite plasma membrane in vesicles or in soluble form via secretory or export pathways. The C-terminus domain is then cleaved off and like other PEXEL-negative exported proteins (PNEPs), it translocates across the parasite plasma membrane (PM) into the parasitophorous vacuole (PV) either via an unidentified translocon or a chaperone, or via the PTEX directly into the RBC cytoplasm. From the PV, the protein is either trafficked across the PTEX into the RBC cytoplasm or directly into nascent budding Maurer's clefts (MC). Soluble PfEMMA1 in the RBC cytoplasm may be incorporated directly into MCs or may be transported by vesicles or J-dots into MCs, where the protein is detected by anti-PfEMMA1 fragment 1 and 2 antibodies but not by anti-PfEMMA1 C-terminus antibodies in permeabilized infected RBCs. From MCs, the protein is delivered to knobs, where it resides on the exofacial surface of RBC plasma membranes and is labeled by anti-PfEMMA1 fragment 1 and 2 antibodies but not by anti-PfEMMA1 C-terminus antibodies in non-permeabilized infected RBCs. Solid lines denote pathways based on experimental evidence; dashed lines denote pathways for other PNEPs as proposed previously (C. Gruring et al., Cell Host Microbe 12, 717-729 (2012), J. A. Boddey and A. F. Cowman Annu Rev Microbiol 67, 243-269 (2013), N. J. Spillman, et al., Annu Rev Biochem 84, 813-841 (2015), T. Spielmann and T. W. Gilberger Trends Parasitol 31, 514-525 (2015), T. Spielmann and T. W. Gilberger Trends Parasitol 26, 6-10 (2010), J. M. Przyborski, et al., Mol Microbiol 101, 1-11 (2016), A. Heiber et al., PLoS Pathog 9, e1003546 (2013), and M. Marti and T. Spielmann, Curr Opin Microbiol 16, 445-451 (2013)).

FIG. 13 is a model for trafficking and surface localization of PfEMMA1. PfEMMA1 is observed initially in trophozoites where it has two possible fates: 1) the C-terminus region is cleaved and the remaining portion of the protein is exported via Maurer's clefts to the exofacial surface of RBC knobs, or 2) the full-length protein is expressed in developing merozoites within schizonts and is localized predominantly at the apex of mature circulating merozoites, where it plays a role in RBC invasion. PfEMMA1 on RBC surfaces interact with endothelial cells and/or host immune cells to evade elimination by the host (e.g. by promoting cytoadherence and sequestration, and down-regulating phagocytes and effector cells). Endoth, endothelium; MC, Maurer's cleft; MZ, merozoite; N, N-terminus; C, C-terminus; PfEMP1, Plasmodium falciparum erythrocyte membrane protein 1; PM, plasma membrane; PPM, parasite plasma membrane; PV, parasitophorous vacuole; PVM, parasitophorous vacuolar membrane; RBC, red blood cell; TM, transmembrane domain.

### DETAILED DESCRIPTION

The invention provides a solution to the urgent need to develop malaria vaccines. P. falciparum blood-stage cDNA lambda phage library was previously screened, and a protein (PF3D7_1134300) that is uniquely recognized by antibodies from a group of malaria-resistant Tanzanian children who controlled parasite density during infection but not by those from a group of susceptible children whose non-immunological risk factors were carefully balanced was identified.

Described herein are findings from a large human cohort, immunolocalized protein, and data indicating that this protein induced growth-limiting antibodies and assessed protection against parasite challenge in a murine model. After adjusting for repeated measures and potential confounders using generalized estimating equations, high levels of monospecific human antibodies (upper 2.5 percentiles) from an actively monitored longitudinal cohort of 450 Tanzanian children 2 to 3.5 years of age (1020 observations) was observed and was associated with an almost 50% reduction in parasite densities on blood smears (124 vs 68 parasites/200 WBC; p=0.04). It was shown by means of immunolocalization studies that the native protein was expressed in trophozoite- and schizont-stage parasites. It was trafficked via Maurer's clefts to the RBC surface membrane despite lacking a recognizable export signal, thereby constituting a PEXEL-negative exported protein (PNEP). In antibody-mediated growth/invasion inhibition assays, murine purified anti-PF3D7_1134300 IgG decreased parasite replication in a dose-dependent fashion by up to 68% compared to pre-immune controls. Mice were immunized with an adjuvanted P. berghei ANKA ortholog of PF3D7_1134300 or adjuvant alone and found that actively immunized mice challenged with 1×10⁴ P. berghei ANKA either survived challenge with P. berghei ANKA or had significantly longer median survival. These data support development of PF3D7_1134300 as a viable vaccine candidate, and present a unique opportunity to elucidate poorly understood mechanisms of protein export and immune protection.

A P. falciparum 3D7 strain blood stage cDNA Lambda Zap phage library (MR4) was screened using a differential approach and identified three antigens encoded by PF3D7_1335100 [MSP-7], PF3D7_1021800 [PfSEA-1] and PF3D7_1134300 that were uniquely recognized by antibodies in plasma from resistant Tanzanian children but not by those from susceptible children. Resistance was defined by a median parasite density of zero (IQR, 25) documented in monthly blood smears obtained from children between 2 and 3.5 years. The disclosure herein characterizes the effector function of murine polyclonal antibodies directed against the immunorelevant portion of PF3D7_1134300. In silico analysis (PlasmoDB.org) predicted that the 6684 bp gene encodes a 263 kDa phospho-protein, contains no introns and has non-human primate, rodent and avian syntenic orthologs. The immunorelevant region was cloned (nt 1,337,023-1,338,945) as well as three overlapping constituent fragments into a eukaryotic expression plasmid (VR2001) and immunized mice to generate antisera. To confirm that PF3D7_1134300 encodes a parasite protein, P. falciparum 3D7 infected and uninfected erythrocytes were probed with antisera, which recognized the protein only in infected erythrocytes. GIA were performed by synchronizing Pf3D7 parasites three times with sorbitol and cultivating them to obtain mature trophozoites that were plated at 1.5% parasitemia (hematocrit 1.0%). Trophozoites were cultured in the presence of heat-inactivated and pre-adsorbed PF3D7_1134300 anti-sera or pre-immune mouse sera for 24 hours and ring stage parasites were enumerated. Antisera directed against the antigen of interest and its constituent fragments inhibited parasite invasion by 31-53% compared to controls (all P<0.01). The data indicated that PF3D7_1134300 is a vaccine candidate for pediatric falciparum malaria.

Bioinformatics analyses of PF3D7_1134300 predict a 6684-base pair single-copy gene that has syntenic orthologs in all human (Plasmodium falciparum, P. vivax, P. ovale, P. malariae, P. knowlesi) and non-human primate, rodent, and avian species studied to date (C. Aurrecoechea et al., Nucleic Acids Res 37, D539-543 (2009)). Herein, it was demonstrated for the first time that the protein encoded by this single-copy gene is exported beyond the parasitophorous vacuole to the exofacial surface of the erythrocyte plasma membrane, despite having no canonical export signals, and is also on the surface of merozoites.

Based on its dual localization, which is a unique feature of this blood-stage malaria protein, the protein was designated as P. falciparum erythrocyte membrane and merozoite antigen 1 (PfEMMA1), and the corresponding gene as PfEMMA1. This blood-stage PEXEL-negative exported protein, which is conserved in all Plasmodium species and lacks homology with mammalian proteins.

Described herein, the utility of PfEMMA1 as a blood-stage malaria subunit vaccine candidate was evaluated. The data here demonstrated that PfEMMA1 was remarkably conserved across field isolates from Africa despite being expressed on erythrocytic and merozoite surfaces; high levels of natural human anti-PfEMMA1 antibodies were associated with significantly lower parasite density (about 50% reduction) in a large immunoepidemiological study; parasite growth was substantially restricted by mouse hyperimmune globulin; and a recombinant protein-based vaccine derived from the Plasmodium berghei ortholog of PfEMMA1 was highly immunogenic, durable and protective in mice leading to eradication of parasites in a subgroup of mice infected with P. berghei ANKA.

Compared to previous vaccine candidates for malaria, the compositions described herein have demonstrated advantages in many different aspects. First, the production level of the recombinant proteins described herein (e.g., Frag1, Frag2 and C-terminus fragments) in E. coli is substantially higher than a longer fragment. In fact, it was demonstrated herein that high level heterologous expression of rPfEMMA1 in bacteria was achieved in inclusion bodies and that it is time- and cost-effective to purify the protein using FPLC with a solubilization and refolding protocol that produces a thermostable product that is amenable to scaling up. Moreover, it is demonstrated herein that PfEMMA1 is highly conserved with limited number of SNPs and low measures of variation (see Example 1), which is an essential prerequisite for a vaccine candidate. In addition, the data here further demonstrate that the native protein is expressed on the surface of both parasitized RBCs (pRBCs) and merozoites, which makes PfEMMA1 a first-in-class vaccine candidate. Being located on both pRBCs and merozoites exposes the protein targets to the host's immune system on the order of hours, thereby maximizing exposure to vaccine-induced immunity during an extended window of susceptibility. Other known blood-stage malaria candidates, however, induce antibodies against only merozoite proteins and are significantly limited by the fact that merozoites circulate for less than ˜2 minutes before they invade RBCs. Indeed, as demonstrated herein (see Example 4), anti-PfEMMA1 antibodies confer heterologous protection against various strains of Plasmodium falciparum malaria (e.g., strains 3D7, Dd2 and W2). It is also demonstrated herein that PfEMMA1 is the only vaccine candidate that has ever prevented mortality in adult mice given a lethal challenge of P. berghei ANKA.

### Polynucleotide Sequences and Encoded Polypeptides

The invention is directed in part to polynucleotides and polypeptides that are useful, for example, for antigens for vaccines against malaria (e.g., P. falciparum malaria, P. berghei).

Fragment 1 (“Frag1”) amino acid sequence of PfEMMA1:
aa 1164-1401 (238aa long)
(SEQ ID NO: 1)
DNVNNNNNKESCDNIKHMRTKSLNFVSRESYGEHKSLDVYQECYVKNNKLINKV
NDKKYEDNNNSYLNEDDNASMQFYEETNSNPYIVDQENNMKNYVNNVLYNNNS
NYYVDSKNYDKSKENAENKSDDILNNENIHTLKDQKKKIQNNNEFISEQADIENIR
NSQEEVYEKEHEPLWVINASNEEKKSYEELIYSDMSSNRVTKNKYSDMNNVEVLL
NEDNLLTTEKYKVQLEKENK
Frag1 nucleotide sequence: nt 3490-4203
(SEQ ID NO: 2)
GATAATGTTAATAATAATAATAATAAAGAAAGTTGTGATAATATTAAACATAT
GAGAACAAAAAGTTTAAATTTTGTAAGTAGAGAATCCTATGGCGAACATAAAA
GTCTAGATGTTTACCAGGAATGTTATGTAAAAAATAATAAACTTATTAATAAG
GTAAATGATAAAAAATATGAGGACAATAATAATTCCTATCTTAATGAAGATGA
TAACGCTAGTATGCAATTTTATGAAGAAACTAATAGTAATCCATATATTGTAGA
CCAGGAAAATAATATGAAAAATTATGTCAATAATGTTTTATATAACAACAATA
GCAATTATTATGTTGATTCAAAGAATTATGATAAATCTAAAGAGAATGCAGAA
AATAAATCAGATGATATATTAAATAATGAAAATATACATACCTTAAAAGATCA
AAAAAAGAAAATACAAAATAATAATGAATTCATTAGTGAACAGGCTGATATA
GAAAATATAAGAAATTCTCAAGAAGAAGTATATGAGAAAGAACACGAACCTT
TGTGGGTAATAAATGCATCTAATGAAGAAAAGAAATCATATGAAGAATTGATA
TACAGCGATATGTCATCTAATCGTGTTACGAAAAATAAATATAGTGATATGAA
TAATGTTGAGGTATTATTAAATGAAGATAATTTATTAACTACTGAAAAATACA
AGGTGCAATTAGAAAAAGAAAATAAA
Fragment 2 (“Frag2”) of PfEMMA1 amino acid sequence:
1364-1600 (237aa long)
(SEQ ID NO: 3)
NRVTKNKYSDMNNVEVLLNEDNLLTTEKYKVQLEKENKMIDMYETVEENINTIK
TENTNDINEEVRNEQKRESINHINDTNINHIIDEYPNDTYNFIKDIECVHNNENNMY
NSIEQYTFYHDTRNNHLVDKNNQNFIFEEEGLNELNFEEKKVYIENNTKDDHKGDS
KTSNLTSLRNTICKSENDHNEKNENTYVVRKGEKGIKRKVSMKKRNEKLNEENYI
NNIYDKMDNHRQNDI
Frag2 nucleotide sequence: nt 4090-4800
(SEQ ID NO: 4)
AATCGTGTTACGAAAAATAAATATAGTGATATGAATAATGTTGAGGTATTATT
AAATGAAGATAATTTATTAACTACTGAAAAATACAAGGTGCAATTAGAAAAAG
AAAATAAAATGATTGATATGTATGAAACGGTAGAGGAGAATATAAATACAATT
AAAACAGAAAATACGAACGACATAAATGAAGAAGTTAGAAACGAACAAAAAA
GAGAAAGTATCAATCATATTAATGATACAAATATAAATCATATAATAGATGAA
TATCCCAATGATACATATAATTTCATAAAAGATATAGAATGTGTACATAACAA
TGAAAATAACATGTACAATTCTATTGAACAATATACATTTTATCATGATACACG
TAATAATCATTTAGTTGATAAAAATAATCAAAATTTTATATTCGAAGAGGAAG
GTTTAAATGAATTGAACTTTGAAGAAAAAAAGGTATATATAGAAAATAATACC
AAGGATGATCACAAGGGAGATAGCAAAACAAGTAACTTAACATCTTTAAGGA
ATACCATATGTAAAAGTGAAAACGATCATAATGAAAAAAATGAAAACACATA
TGTGGTTAGAAAAGGCGAAAAAGGAATTAAACGTAAGGTTTCCATGAAGAAA
AGAAATGAAAAGCTAAATGAAGAAAATTATATTAATAATATATACGATAAAAT
GGATAACCATAGACAAAATGATATT
C-Terminal fragment (“C-Term”) of PfEMMA1 amino acid
sequence: aa 2140-2223 (84aa long)
(SEQ ID NO: 5)
LKINKKVPKYKLENMNINSPNIPYTYLFICDGSNYLCINDNSLNNEVYENKMKLNN
IIGYYHYINLNRLTYYLEKVNANFVYNH
C-Term nucleotide sequence: nt 6418-6669
(SEQ ID NO: 6)
TTAAAGATAAATAAGAAAGTTCCTAAATATAAATTGGAAAATATGAATATTAA
CTCACCAAATATTCCATATACATATCTTTTTATATGTGATGGAAGTAACTATTT
ATGTATTAATGACAATTCATTAAATAACGAGGTATATGAAAACAAGATGAAAT
TGAACAATATCATTGGATATTACCATTATATTAATTTGAATAGATTAACATATT
ATTTAGAAAAGGTAAATGCTAATTTTGTTTATAACCAT
Amino acid sequence of PfEMMA1 (PF11_0354)
(SEQ ID NO: 21)
MRSKSISYFLFFKKNKKKNDSCDSVIISSNKNLSIQLSKGEDDEKNEINEEKSYIKNE
DVYKKEKLKKKKENKENNKKKDKNEVVYDYHDISNDATSDYVNNYKVYEMNT
CNIKKKRESFFKKINILQKYKNYKIRKAASTFHTIGHKTSFSGTDDEIENNQKKQKK
YKIKISEWKDDKSHTFHKKNDILVFDKMDKNKKFKIDNNKNNQINIDNEERVNKN
YPMATNVQNFNIKYTSIDVTNDEYIIDSNKPEGSIMSTDKKNNKLNYNNDTYDVD
KSSDINKLGNIKKNKFDIITKTTHNINNNVNNIHNYMMYTNKENIKININHGNLNGR
EQNNYDEERKANVYEIFENAKKLEPNNININTEEHIHISEPSIPFDMKDHKNDINEK
DIILKLMYNNNGIYFDDDDENHKNLLYKNKDTHVKHLNNKFNHNFIIYNDREEGV
NQKHAQKKLKKKNTILNKNENEDINHNSFKRPLSNTNICYKDKDDKIKNGSNKYD
ILNNDYSNEHEKNKYNDHITKNKRNQSANEVKSNNNDNHNNKKNNNFNININDS
YSTNINRNQNVMINDVNDVIKDPNMQENTQGDDEGGIINKYLINPIYNLFLRANEEI
QNSNSTNNKLKMNNITKSYTNELQKTYKSMYDINDISNKRKINNKDIRGTNLYNT
KLCNNKLYNSNPYNMIPYNINTYNNNNNNKETCTSINIKHSENKYPFNKSHVNSY
MKNTNHLPHRNAITSNNRNNEEYEKEKEKDRNITNGNNNYLVEYNNSCIPPPLKK
MIPIDGVRNKSINKLNNVTNTQRTSSVSYTNKNIDENSFDMPIINGIRESKYISNNNN
INGNSIGFNSSKLDNYHHQSMNVNESYPLKNMMKNNYIEHNYDDKNNIFLVKNY
EDTYSNIHNGIHENSMLKNYNLKKACTFHGYSRNHQKNMYTEENLNINQKKNYS
HYHNNGTVLKPLVNTNNVAVNEFADINLSAQKRLHSLKSMGYEDKSMENYRNKI
YNNINNNNNNNNDNNIYNDNEYCQYNNSYCFDHSDLKNMFPLNHQNSKLLTHSN
NKNSFFNGINVESKHHLANPEIKTFAHNSYPILNQGLINCNPLQCLGYDSNQRNKH
NVVYIKKNEYLNKNIGSIINVLKREGLRKISTHNGKFESFSNMDNKNVYMEGLNIQ

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

MIDMYETVEENINTIKTENTNDINEEVRNEQKRESINHIND
TNINHIIDEYPNDTYNFIKDIECVHNNENNMYNSIEQYTFYHDTRNNHLVDKNNQNFIFE
EEGLNELNFEEKKVYIENNTKDDHKGDSKTSNLTSLRNTICKSENDHNEKNENTYVVRK
GEKGIKRKVSMKKRNEKLNEENYINNIYDKMDNHRQNDITKKENDEENYILYNNVKV
NYDEYIENGNKIKITEESLNVFYKENQNEEDSSTKKLNSTSKIKRANKGKTKKKNV
ITRVHKTKQKIEYVTNSFNKSSKGENSEIGKIGGRSKSLLTHSKKVSERNKNKIEKIN
DTNSKIIKGKKSNSQSKLGKDTKIRGKSKTGEYIKNKDLRKKSNEKNKTVMDNINT
INNSSVSNLKSKKHKLKKKKKKNISMENINKNITNEFCSMERKGTVLLSNMSIKKI
DNANSCTLNEPLEENTLNYESNNNCSNSNLSKDKEKDRNILCNKYYSDEETNSLN
KMYTSNIPEISNYYKEIQAINYILSNINNPNFLNSLELNDLINIEKKFINENIYINKQIIA
CNVKNEKSNDEMVEKNERKVDEEKGEDEQEIKAKENNNKEENQDNENNNKEEN
HDNENNNKEENQDNENNNKEENQDNENNNKEENQDNENNNKEENQKNENGIIY
DSRFSIIYLEHDLIYLKKNNLKVILNVLLSNVYCFFEIKLTIILLNFFISNNCQWSFSLF
PLSLINKLIHKFS [symbol] [symbol] [symbol] [symbol] [symbol] [symbol] [symbol]
HIYE,
Sequence Length: 2227
Bold/Italic: Frag1 (aa 1164-1401)
Underlined: Frag2 (aa 1364-1600)
Bold/Double underlined: C-terminal fragment (aa 2140-2223)
Coding Nucleic acid sequence gene PfEMMA1 (PF11_0354--old ID)
(PF3D7_1134300)
(SEQ ID NO: 22)
ATGAGATCGAAATCCATTTCGTATTTCTTATTTTTTAAAAAAAACAAAAAGAAA
AATGATTCTTGTGATAGTGTCATAATATCTAGCAATAAGAATTTATCCATTCAA
TTATCGAAAGGTGAGGATGATGAAAAAAATGAAATAAATGAGGAAAAGAGTT
ATATAAAAAATGAAGATGTATATAAAAAGGAAAAATTAAAAAAGAAGAAAGA
AAACAAGGAAAATAATAAAAAGAAAGATAAAAATGAAGTAGTATATGATTAT
CATGACATTTCAAATGATGCTACTAGTGATTATGTTAATAATTATAAAGTATAT
GAAATGAATACTTGTAATATAAAAAAGAAGAGAGAAAGTTTTTTTAAAAAAAT
TAATATTTTACAAAAATATAAAAATTACAAAATTAGAAAGGCAGCTAGTACCT
TTCATACCATAGGACATAAAACATCTTTTTCTGGTACAGATGATGAAATAGAA
AATAATCAAAAGAAACAAAAAAAATATAAAATAAAAATTTCTGAATGGAAGG
ATGATAAATCACATACTTTTCATAAAAAAAATGACATATTGGTATTTGATAAG
ATGGATAAAAATAAAAAATTTAAAATTGATAACAACAAAAACAATCAAATTA
ATATAGATAATGAAGAAAGAGTTAATAAAAATTATCCTATGGCTACTAATGTA
CAAAATTTTAATATAAAATATACATCAATAGATGTAACAAATGACGAATATAT
TATAGATTCTAATAAACCTGAAGGTTCTATTATGTCTACAGATAAAAAGAATA
ATAAACTTAATTATAATAATGATACATATGATGTAGACAAAAGCTCTGATATA
AATAAGTTAGGTAATATAAAAAAGAATAAATTTGATATTATTACTAAAACAAC
ACATAATATTAATAATAATGTAAATAATATACATAATTATATGATGTATACAA
ATAAAGAAAATATAAAAATAAATATAAATCATGGAAATCTAAATGGAAGAGA
ACAAAACAATTATGATGAAGAAAGGAAAGCAAATGTTTATGAAATATTTGAAA
ATGCAAAAAAATTAGAACCTAATAATATTAATATCAACACAGAAGAACATATT
CATATTAGTGAACCCAGCATACCATTTGATATGAAGGATCATAAAAATGATAT
AAATGAAAAAGATATAATATTAAAATTGATGTATAACAATAACGGTATTTATT
TTGATGATGATGATGAAAATCACAAGAATTTATTATACAAAAATAAAGATACA
CATGTAAAACATTTAAATAATAAATTTAACCATAATTTTATTATATATAATGAT
CGCGAAGAAGGGGTAAATCAGAAACACGCACAAAAAAAATTAAAAAAAAAA
AATACTATTCTTAACAAAAACGAAAATGAAGATATTAATCATAATAGTTTCAA
AAGACCTTTATCTAATACGAATATATGTTATAAGGACAAAGATGATAAAATTA
AAAATGGTTCTAATAAGTATGATATATTAAATAATGACTATTCTAATGAACAC
GAAAAAAATAAATATAATGATCATATAACAAAAAATAAAAGAAATCAATCAG
CAAATGAAGTAAAATCTAATAATAATGATAACCACAATAATAAAAAAAATAAT
AATTTTAATATTAATATTAATGATTCATATTCTACAAATATAAATAGAAACCAA
AATGTGATGATAAATGATGTAAACGATGTTATTAAGGATCCAAATATGCAGGA
AAATACACAAGGTGATGACGAAGGTGGTATTATAAACAAATATTTAATTAACC
CTATTTACAATTTATTTCTACGTGCTAATGAAGAAATACAAAATTCAAATAGTA
CAAACAATAAATTAAAAATGAATAATATAACAAAAAGTTATACAAACGAACT
ACAAAAGACATATAAAAGTATGTACGATATAAATGATATATCAAATAAGAGA
AAAATTAATAATAAAGATATACGTGGAACTAATTTGTATAACACCAAATTATG
TAATAATAAATTATATAATTCGAATCCATATAATATGATTCCATATAATATAAA
CACATATAATAATAATAATAATAATAAGGAAACTTGTACCAGCATAAATATCA
AACATTCCGAAAATAAATATCCCTTCAATAAATCTCATGTAAACTCATATATGA
AAAATACAAATCATCTTCCTCATAGAAATGCGATTACATCAAATAATAGAAAC
AATGAAGAATATGAGAAAGAAAAAGAAAAAGATCGTAACATTACTAATGGGA
ACAATAATTATTTGGTTGAATATAATAATTCTTGTATACCTCCACCACTCAAAA
AAATGATACCAATAGATGGTGTGAGAAATAAAAGTATAAATAAATTAAATAAT
GTAACTAATACGCAACGTACATCAAGTGTTTCATATACGAATAAGAATATTGA
TGAGAATTCGTTTGATATGCCTATAATAAATGGAATAAGAGAATCTAAATATA
TAAGTAATAATAATAATATTAATGGTAATTCCATTGGTTTTAATTCATCTAAGT
TAGATAATTATCATCACCAATCTATGAATGTGAATGAATCTTATCCTCTAAAAA
ATATGATGAAAAATAATTATATTGAACATAATTATGATGATAAAAATAATATT
TTCCTTGTTAAAAATTATGAAGATACATATTCAAATATTCATAATGGCATACAT
GAAAATAGCATGCTAAAAAATTATAATTTAAAAAAAGCGTGCACTTTTCATGG
GTACTCTAGAAATCACCAAAAAAATATGTATACGGAAGAAAATTTAAATATTA
ATCAAAAAAAGAATTATAGTCATTATCATAATAATGGAACGGTATTAAAACCT
TTGGTAAATACTAATAATGTTGCAGTGAACGAATTTGCAGATATTAATTTATCG
GCTCAAAAAAGATTACATAGTTTAAAAAGTATGGGGTACGAGGATAAGAGTAT
GGAAAATTACAGAAACAAAATATACAACAACATCAATAATAATAATAATAAT
AATAATGATAATAATATATATAATGATAATGAATATTGTCAGTATAATAATAG
TTATTGTTTCGATCATAGTGATTTAAAAAATATGTTTCCATTAAATCATCAGAA
TAGCAAGTTATTAACACATAGTAATAATAAAAATTCATTTTTTAACGGAATAA
ATGTAGAATCGAAACATCATTTAGCAAATCCTGAAATAAAAACATTTGCACAC
AATAGTTATCCTATATTAAATCAAGGTTTAATAAATTGTAACCCCTTACAATGC
TTGGGTTATGATTCAAATCAAAGGAATAAGCATAATGTAGTATACATAAAAAA
AAATGAATACCTTAATAAAAACATTGGCTCTATTATAAATGTTCTTAAAAGAG
AAGGACTAAGAAAAATTTCTACACATAATGGAAAATTCGAATCATTTAGTAAT
ATGGATAATAAAAATGTATATATGGAAGGACTAAACATACAA

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

ATGATTGATATG
TATGAAACGGTAGAGGAGAATATAAATACAATTAAAACAGAAAATACGAACG
ACATAAATGAAGAAGTTAGAAACGAACAAAAAAGAGAAAGTATCAATCATAT
TAATGATACAAATATAAATCATATAATAGATGAATATCCCAATGATACATATA
ATTTCATAAAAGATATAGAATGTGTACATAACAATGAAAATAACATGTACAAT
TCTATTGAACAATATACATTTTATCATGATACACGTAATAATCATTTAGTTGAT
AAAAATAATCAAAATTTTATATTCGAAGAGGAAGGTTTAAATGAATTGAACTT
TGAAGAAAAAAAGGTATATATAGAAAATAATACCAAGGATGATCACAAGGGA
GATAGCAAAACAAGTAACTTAACATCTTTAAGGAATACCATATGTAAAAGTGA
AAACGATCATAATGAAAAAAATGAAAACACATATGTGGTTAGAAAAGGCGAA
AAAGGAATTAAACGTAAGGTTTCCATGAAGAAAAGAAATGAAAAGCTAAATG
AAGAAAATTATATTAATAATATATACGATAAAATGGATAACCATAGACAAAAT
GATATTACAAAAAAAGAAAATGACGAAGAAAATTATATTTTGTACAACAACGT
AAAGGTTAATTATGATGAATATATAGAAAATGGAAATAAAATAAAAATAACG
GAAGAATCATTAAATGTCTTTTATAAAGAAAATCAAAATGAGGAAGATTCTTC
TACAAAAAAGTTGAATAGTACAAGTAAAATAAAACGTGCAAACAAAGGGAAA
ACAAAAAAAAAGAATGTTATCACAAGGGTACATAAAACAAAACAAAAAATTG
AATATGTTACAAATAGTTTTAATAAATCTTCCAAAGGTGAAAATTCAGAAATA
GGAAAAATTGGAGGTAGGAGTAAATCATTATTAACACACAGCAAGAAAGTTA
GTGAACGAAATAAAAATAAAATAGAAAAAATTAATGATACAAATTCAAAGAT
AATAAAAGGAAAAAAGAGTAATAGCCAAAGCAAACTTGGGAAGGATACAAAA
ATTAGAGGGAAATCAAAAACTGGGGAATATATAAAAAATAAAGATTTAAGAA
AAAAATCTAACGAAAAAAACAAAACAGTGATGGATAATATAAATACTATAAA
TAATTCTTCAGTATCTAACCTAAAAAGCAAAAAACATAAATTGAAAAAAAAAA
AAAAAAAAAATATATCTATGGAAAATATAAATAAAAATATAACAAATGAATTT
TGTTCTATGGAAAGAAAAGGAACCGTTCTATTATCTAATATGAGTATTAAGAA
GATTGATAATGCAAATAGTTGTACATTAAATGAACCATTAGAGGAAAATACCT
TAAATTATGAAAGTAATAATAACTGTAGTAATAGTAATTTATCTAAGGATAAA
GAAAAAGATAGAAATATATTGTGTAATAAATATTATAGTGATGAGGAAACAAA
CTCTTTAAACAAAATGTATACATCGAATATACCAGAAATAAGTAATTATTATA
AGGAAATTCAAGCAATTAATTACATATTAAGTAATATTAATAATCCAAATTTTT
TAAATTCCCTCGAACTGAATGATTTAATAAATATTGAAAAAAAATTTATTAACG
AAAATATATATATTAATAAGCAGATAATAGCCTGTAATGTAAAAAATGAAAAA
TCAAATGATGAGATGGTCGAGAAAAATGAACGCAAAGTGGATGAAGAAAAAG
GAGAAGACGAACAAGAAATAAAAGCAAAGGAAAATAATAATAAAGAAGAAA
ACCAAGATAATGAAAATAATAATAAAGAAGAAAACCATGATAATGAAAATAA
TAATAAAGAAGAAAATCAAGATAATGAAAATAATAATAAAGAAGAAAACCAA
GATAATGAAAATAATAATAAAGAAGAAAATCAAGATAATGAAAATAATAATA
AAGAAGAAAACCAAAAAAATGAAAATGGTATTATTTATGATAGCAGGTTTAGT
ATTATCTATTTAGAACACGATTTAATATATTTAAAAAAAAATAATTTAAAAGTG
ATACTTAATGTTTTGCTGTCAAATGTGTATTGCTTTTTTGAAATTAAATTAACCA
TAATATTGTTAAATTTCTTTATATCTAATAATTGTCAATGGAGTTTCAGTTTATT
TCCCCTTTCATTAATTAATAAATTAATACATAAATTCAGT

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

CATATATATGAATAA,
Sequence Length: 6684 bp
Bold/Italic: Frag1 (nt 3490-4203)
Underlined: Frag2 (nt 4090-4800)
Bold/Double underlined: C-terminus fragment (nt 6418-6669)
N-terminal fragment 1 of PfEMMA1 (NT-1)
(SEQ ID NO: 23)
MRSKSISYFLFFKKNKKKNDSCDSVIISSNKNLSIQLSKGEDDEKNEINEEKSYIKNE
DVYKKEKLKKKKENKENNKKKDKNEVVYDYHDISNDATSDYVNNYKVYEMNT
CNIKKKRESFFKKINILQKYKNYKIRKAASTFHTIGHKTSFSGTDDEIENNQKKQKK
YKIKISEWKDDKSHTFHKKNDILVFDKMDKNKKFKIDNNKNNQINIDNEERVNKN
YPMATNVQNFNIKYTSIDVTNDEYIIDSNKPEGSIMSTDKKNNKLNYNNDTYDVD
N-terminal fragment 2 of PfEMMA1 (NT-2)
(SEQ ID NO: 24)
KSSDINKLGNIKKNKFDIITKTTHNINNNVNNIHNYMMYTNKENIKININHGNLNGR
EQNNYDEERKANVYEIFENAKKLEPNNININTEEHIHISEPSIPFDMKDHKNDINEK
DIILKLMYNNNGIYFDDDDENHKNLLYKNKDTHVKHLNNKFNHNFIIYNDREEGV
NQKHAQKKLKKKNTILNKNENEDINHNSFKRPLSNTNICYKDKDDKIKNGSNKYD
ILNNDYSNEHEKNKYNDHITKNKRNQSANEVKSNNNDNHNNKKNNNFNININD
N-terminal fragment 3 of PfEMMA1 (NT-3)
(SEQ ID NO: 25)
SYSTNINRNQNVMINDVNDVIKDPNMQENTQGDDEGGIINKYLINPIYNLFLRANE
EIQNSNSTNNKLKMNNITKSYTNELQKTYKSMYDINDISNKRKINNKDIRGTNLYN
TKLCNNKLYNSNPYNMIPYNINTYNNNNNNKETCTSINIKHSENKYPFNKSHVNSY
MKNTNHLPHRNAITSNNRNNEEYEKEKEKDRNITNGNNNYLVEYNNSCIPPPLKK
MIPIDGVRNKSINKLNNVTNTQRTSSVSYTNKNIDENSFDMPIINGIRESKYISNNNN
N-terminal fragment 4 of PfEMMA1 (NT-4)
(SEQ ID NO: 26)
INGNSIGFNSSKLDNYHHQSMNVNESYPLKNMMKNNYIEHNYDDKNNIFLVKNY
EDTYSNIHNGIHENSMLKNYNLKKACTFHGYSRNHQKNMYTEENLNINQKKNYS
HYHNNGTVLKPLVNTNNVAVNEFADINLSAQKRLHSLKSMGYEDKSMENYRNKI
YNNINNNNNNNNDNNIYNDNEYCQYNNSYCFDHSDLKNMFPLNHQNSKLLTHSN
NKNSFFNGINVESKHHLANPEIKTFAHNSYPILNQGLINCNPLQCLGYDSNQRNKH
NVVYIKKNEYLNKNIGSIINVLKREGLRKISTHNGKFESFSNMDNKNVYMEGLNIQ

The invention encompasses “fragments,” “antigenic fragments” or “antigens,” or “antigenic peptides” of SEQ ID NO: 1, 3, 5 or 21. Such fragments and peptides represent portions of the polypeptide that have, for example, specific immunogenic or binding properties. An antigenic fragment can be between 3-10 amino acids, 10-20 amino acids, 20-40 amino acids, 40-56 amino acids in length or even longer. Amino acid sequences having at least 70% amino acid identity, preferably at least 80% amino acid identity, more preferably at least 90% identity, and most preferably 95% identity to the fragments described herein are also included within the scope of the present invention. Exemplary antigenic fragments of SEQ ID NO: 1, 3 or 5 are listed in the tables below. Exemplary antigenic fragments of SEQ ID NO: 21 include SEQ ID NOs: 1, 3, 5, 23, 24, 25, and 26.

TABLE 1

| | Exemplary antigenic fragments of |
|---|---|
| SEQ ID NO | Frag 1 |
| SEQ ID NO: 7 | DNVNNNNNKESCDNIKHMRTKSLNFVSRESYGEHKS |
| | LDVYQE |
| SEQ ID NO: 8 | CYVKNNKLINKVNDKKYEDNNNSYLNEDDNASMQFY |
| | EETNS |
| SEQ ID NO: 9 | NPYIVDQENNMKNYVNNVLYNNNSNYYVDSKNYDKS |
| | KENAE |
| SEQ ID NO: 10 | NKSDDILNNENIHTLKDQKKKIQNNNEFISEQADIE |
| | NIRNSQEE |
| SEQ ID NO: 11 | VYEKEHEPLWVINASNEEKKSYEELIYSDMSSNRVT |
| | KNKYSD |
| SEQ ID NO: 12 | MNNVEVLLNEDNLLTTEKYKVQLEKENK |

TABLE 2

| | Exemplary antigenic fragments of |
|---|---|
| SEQ ID NO | Frag2 |
| SEQ ID NO: 13 | NRVTKNKYSDMNNVEVLLNEDNLLTTEKYKVQLEKE |
| | NKMID |
| SEQ ID NO: 14 | MYETVEENINTIKTENTNDINEEVRNEQKRESINHI |
| | NDTNINHII |
| SEQ ID NO: 15 | DEYPNDTYNFIKDIECVHNNENNMYNSIEQYTFYHD |
| | TRNNHL |
| SEQ ID NO: 16 | VDKNNQNFIFEEEGLNELNFEEKKVYIENNTKDDHK |
| | GDSKTSN |
| SEQ ID NO: 17 | LTSLRNTICKSENDHNEKNENTYVVRKGEKGIKRKV |
| | SMKKRN |
| SEQ ID NO: 18 | EKLNEENYINNIYDKMDNHRQNDI |

TABLE 3

| | Exemplary antigenic fragments of |
|---|---|
| SEQ ID NO | C-terminus fragment |
| SEQ ID NO: 19 | LKINKKVPKYKLENMNINSPNIPYTYLFICDGSNYL |
| | CINDNSLN |
| SEQ ID NO: 20 | NEVYENKMKLNNIIGYYHYINLNRLTYYLEKVNANF |
| | VYNH |

TABLE 4

| SEQ ID NO | Corresponding Sequence |
|---|---|
| 1 | Frag1 aa (238 AA IN LENGTH-- (AA 1164-1401) |
| 2 | Frag1 nt 3490-4203 |
| 3 | Frag2 aa (237 AA IN LENGTH - AA 1364-1600) |
| 4 | Frag2 nt (aa 4090-4800) |
| 5 | C-term aa (84 AA IN LENGTH--AA 2140-2223) |
| 6 | C-term nt 6418-6669 |
| 7-12 | 6 antigenic fragments of Frag1 AA |
| | 1) 1164-1209 |
| | 2) 1199-1249 |
| | 3) 1239-1289 |
| | 4) 1279-1329 |
| | 5) 1319-1369 |
| | 6) 1359-1401 |
| 13-18 | 6 antigenic fragments of Frag2 AA |
| | 1) 1364-1409 |
| | 2) 1399-1449 |
| | 3) 1439-1489 |
| | 4) 1479-1529 |
| | 5) 1519-1569 |
| | 6) 1559-1600 |
| 19-20 | 2 antigenic fragments of C-term AA |
| | 1) 2140-2187 |
| | 2) 2177-2223 |
| 21 | Full length FfEMMA1 AA 1-2227 |
| 22 | Full length FfEMMA nt 1- 6684 |

Furthermore, the invention encompasses fragments and derivatives of the nucleic acid sequences described herein, as well as fragments and portions of the amino acid sequences described herein.

A “polynucleotide” is a nucleic acid polymer of RNA, DNA, modified RNA or DNA, or RNA or DNA mimetics (such as peptide nucleic acids (PNAs)), and derivatives thereof, and homologues thereof. Thus, polynucleotides include polymers composed of naturally occurring nucleobases, sugars and covalent inter-nucleoside (backbone) linkages as well as polymers having non-naturally-occurring portions that function similarly. Such modified or substituted nucleic acid polymers are well known in the art and for the purposes of the present invention, are referred to as “analogues.” Oligonucleotides are generally short polynucleotides from about 10 to up to about 160 or 200 nucleotides.

A “variant polynucleotide” or a “variant nucleic acid sequence” means a polynucleotide having at least about 60% nucleic acid sequence identity, more preferably at least about 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% nucleic acid sequence identity and yet more preferably at least about 99% nucleic acid sequence identity with the nucleic acid sequence of SEQ ID NO: 2, 4, 6 or 22. Variants do not encompass the native nucleotide sequence. Other variant polynucleotides include those that differ from SEQ ID NO: 2, 4, 6 or 22, but because of the redundancy of the genetic code, encode a polypeptide of SEQ ID No: 1, 3, 5, or 21, respectively, or fragments of variants thereof.

Ordinarily, variant polynucleotides are at least about 8 nucleotides in length, often at least about 9, 10, 11, 12, 13, 14, 15, 16, 17, 18, 19, 20, 21, 22, 23, 24, 25, 26, 27, 28, 29 30, 35, 40, 45, 50, 55, 60 nucleotides in length, or even about 75-200 nucleotides in length, or more.

In general, a polypeptide variant preserves antigenic function and includes any variant in which residues at a particular position in the sequence have been substituted by other amino acids, and further includes the possibility of inserting an additional residue or residues between two residues of the parent polypeptide as well as the possibility of deleting one or more residues from the parent sequence.

“A polypeptide variant” means a polypeptide having at least about 70% amino acid sequence identity with an amino acid sequence of SEQ ID NOs: 1, 3, 5, 7, 8, 9, 10, 11, 12, 13, 14, 15, 16, 17, 18, 19, 20, 23, 24, 25 or 26. For example, polypeptide variants include those wherein one or more amino acid residues are added or deleted at the N- or C-terminus of the full-length native amino acid sequence. A polypeptide variant will have at least about 71%-75% amino acid sequence identity; at least about 76%-79% amino acid sequence identity; at least about 80% amino acid sequence identity, at least about 81% amino acid sequence identity, at least about 82%, 83%, 84%, 85%, 86%, 87%, 88%, 89%, 90%, 91%, 92%, 93%, 94%, 95%, 96%, 97%, 98% amino acid sequence identity and at least about 99% amino acid sequence identity with a full-length sequence. Ordinarily, variant polypeptides are at least about 10 amino acids in length, often at least about 20 amino acids in length, more often at least about 30, 40, 50, 60, 70, 80, 90, 100, 150, 200, or 300 amino acids in length, or more.

Useful conservative substitutions are shown in Table 4 below. Conservative substitutions whereby an amino acid of one class is replaced with another amino acid of the same type fall within the scope of the subject invention so long as the substitution does not materially alter the biological activity of the compound.

TABLE 4
Conservative amino acid substitutions.
Exemplary substitutions

| Original | | Preferred |
|---|---|---|
| residue | Exemplary substitutions | substitutions |
| Cys (C) | Ser | Ser |
| Gln (Q) | Asn | Asn |
| Glu (E) | Asp | Asp |
| Gly (G) | Pro, Ala | Ala |
| His (H) | Asn, Gln, Lys, Arg | Arg |
| Ile (I) | Leu, Val, Met, Ala, Phe, Norleucine | Leu |
| Leu (L) | Norleucine, Ile, Val, Met, Ala, Phe | Ile |
| Lys (K) | Arg, Gln, Asn | Arg |
| Met (M) | Leu, Phe, Ile | Leu |
| Phe (F) | Leu, Val, Ile, Ala, Tyr | Leu |
| Pro (P) | Ala | Ala |
| Ser (S) | Thr | Thr |
| Thr (T) | Ser | Ser |
| Trp (W) | Tyr, Phe | Tyr |
| Typ (Y) | Trp, Phe, Thr, Ser | Phe |
| Val (V) | Ile, Leu, Met, Phe, Ala, Norleucine | Leu |

The polypeptides of the invention can be either synthesized in vitro or expressed recombinantly from the polynucleotide sequences. Because of redundancy in the genetic code, the sequences need not be identical to practice the invention. Polynucleotide and polypeptide sequence identities can be from 70%-100%, such as 70%, 75%, 80%, 81%, 82%, 83%, 84%, 85%, 86%, 87%, 88%, 89%, 90%, 91%, 92%, 93%, 94%, 95%, 96%, 97%, 98%, 99% and 100%.

The polypeptides of the invention can be readily synthesized in vitro using polypeptide chemistry. For example, polypeptide synthesis can be carried out in a stepwise manner on a solid phase support using an automated polypeptide synthesizer, such as a Rainin Symphony Peptide Synthesizer, Advanced Chemtech Peptide Synthesizer, Argonaut Parallel Synthesis System, or an Applied Biosystems Peptide Synthesizer. The peptide synthesizer instrument combines the Fmoc chemistry with HOBt/HBTU/DIEA activation to perform solid-phase peptide synthesis.

The side chains of many amino acids contain chemically reactive groups, such as amines, alcohols, or thiols. These side chains must be additionally protected to prevent undesired side-reactions during the coupling step. Side chain protecting groups that are base-stable, more preferably, both base-stable and acid-labile are most useful.

“Percent (%) nucleic acid sequence identity” with respect to nucleic acid sequences is defined as the percentage of nucleotides in a candidate sequence that are identical with the nucleotides in the sequence of interest, after aligning the sequences and introducing gaps, if necessary, to achieve the maximum percent sequence identity. Alignment for purposes of determining % nucleic acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

“Consisting essentially of a polynucleotide having a % sequence identity” means that the polynucleotide does not substantially differ in length, but may differ substantially in sequence. Thus, a polynucleotide “A” consisting essentially of a polynucleotide having at least 80% sequence identity to a known sequence “B” of 100 nucleotides means that polynucleotide “A” is about 100 nts long, but up to 20 nts can vary from the “B” sequence. The polynucleotide sequence in question can be longer or shorter due to modification of the termini, such as, for example, the addition of 1-15 nucleotides to produce specific types of probes, primers and other molecular tools, etc., such as the case of when substantially non-identical sequences are added to create intended secondary structures. Such non-identical nucleotides are not considered in the calculation of sequence identity when the sequence is modified by “consisting essentially of.”

### Vaccine Compositions

The invention described herein is further directed to an immunogenic composition, e.g., a vaccine composition capable of blocking P. falciparum infection, for example, a peptide vaccine or a DNA vaccine capable of reducing or inhibiting human to mosquito transmission (i.e., transmission blocking, which refers to protection achieved by directly blocking or inhibiting gametocytes in the human circulation or by inhibiting the formation of oocysts in the mosquito midgut after the mosquito ingests gametocytes, thereby preventing replication of the parasite sexual forms), inhibiting growth of parasites in RBCs, and/or eliciting an antibody or cellular immune response to the vaccine antigen(s). The vaccine composition comprises one or more of the polypeptides, the nucleic acid sequences, or antigens thereof, as described herein.

A person skilled in the art will be able to select preferred peptides, polypeptides, nucleic acid sequences or combination of thereof by testing vaccine candidates as described herein. The Peptides with the desired activity are then identified and formulated as a vaccine, e.g., together with an immunologically inactive excipient and/or an adjuvant. A suitable vaccine will preferably contain between 1 and 20 peptides, more preferably 2, 3, 4, 5, 6, 7, 8, 9, 10, 11, 12, 13, 14, 15, 16, 17, 18, 19, or 20 different peptides, further preferred 6, 7, 8, 9, 10 11, 12, 13, or 14 different peptides, and most preferably 12, 13 or 14 different peptides. Alternatively, a suitable vaccine will preferably contain between 1 and 20 nucleic acid sequences, more preferably 2, 3, 4, 5, 6, 7, 8, 9, 10, 11, 12, 13, 14, 15, 16, 17, 18, 19, or 20 different nucleic acid sequences, further preferred 6, 7, 8, 9, 10 11, 12, 13, or 14 different nucleic acid sequences, and most preferably 12, 13 or 14 different nucleic acid sequences.

Such a vaccine is used for active immunization of a mammal, for example, a human who risks being exposed to one or more Plasmodium antigens (for example, due to travel within a region in which malaria is prevalent). For example, the vaccine can contain at least one antigen selected from the group consisting of: 1) an antigen comprising a polypeptide having at least 70% (e.g. 70%, 75%, 80%, 85%, 90%, 91%, 92%, 93%, 94%, 95%, 96%, 97%, 98%, 99% or 100%) sequence identity with an amino acid sequence selected from the group consisting of SEQ ID NOs: 1, 3, 5, 7, 8, 9, 10, 11, 12, 13, 14, 15, 16, 17, 18, 19, 20, 23, 24, 25, 26, and fragments thereof; 2) an antigen comprising a polypeptide having at least 70% to 99% (e.g., 70%, 75%, 80%, 85%, 90%, 91%, 92%, 93%, 94%, 95%, 96%, 97%, 98% or 99%) sequence identity to an amino acid sequence selected from the group consisting of SEQ ID NOs: 1, 3, 5, 7, 8, 9, 10, 11, 12, 13, 14, 15, 16, 17, 18, 19, 20, 23, 24, 25, 26, and fragment thereof; 3) an antigen comprising a polypeptide consisting essentially of the amino acid sequence of SEQ ID NO: 1, 3, 5, 7, 8, 9, 10, 11, 12, 13, 14, 15, 16, 17, 18, 19, 20, 23, 24, 25, 26, or fragment thereof; 4) an antigen consisting of the amino acid sequence of SEQ ID NO: 1, 3, 5, 7, 8, 9, 10, 11, 12, 13, 14, 15, 16, 17, 18, 19, 20, 23, 24, 25, 26, or fragment thereof; 5) a nucleic acid sequence having at least 70% (e.g. 70%, 75%, 80%, 85%, 90%, 91%, 92%, 93%, 94%, 95%, 96%, 97%, 98%, 99% or 100%) sequence identity with a nucleic acid sequence encoding any one of the peptides listed above; 6) a nucleic acid sequence having at least 70% to 99% (e.g., 70%, 75%, 80%, 85%, 90%, 91%, 92%, 93%, 94%, 95%, 96%, 97%, 98% or 99%) sequence identity to a nucleic acid sequence encoding the listed polypeptides; 7) a nucleic acid sequence consisting essentially of the nucleic acid sequence described above; and 8) a nucleic acid sequence consisting of the nucleic acid sequence described above. A fragment of these polypeptides can be approximately 8-56 amino acid residues, such as 8, 9, 10, 20, 30, 40, 50, 51, 52, 53, 54, 55, and 56 residues. A fragment of these nucleic acid sequences can be approximately 10-300 nucleotides, such as 10, 20, 30, 40, 50, 60, 70, 80, 90, 100, 110, 120, 130, 140, 150, 160, 170, 180, 190, 200, 210, 220, 230, 240, 250, 260, 270, 280, 290, or 300 nucleotides.

Alternatively, if passive immunization is desired, one can administer one or more antibodies to the following antigens (as a vaccination): 1) a polypeptide having at least 70% (e.g. 70%, 75%, 80%, 85%, 90%, 91%, 92%, 93%, 94%, 95%, 96%, 97%, 98%, 99% or 100%) sequence identity with an amino acid sequence selected from the group consisting of SEQ ID NOs: 1, 3, 5, 7, 8, 9, 10, 11, 12, 13, 14, 15, 16, 17, 18, 19, 20, 23, 24, 25, 26, and fragment thereof; 2) a polypeptide having at least 70% to 99% (e.g., 70%, 75%, 80%, 85%, 90%, 91%, 92%, 93%, 94%, 95%, 96%, 97%, 98% or 99%) sequence identity to an amino acid sequence selected from the group consisting of SEQ ID NOs: 1, 3, 5, 7, 8, 9, 10, 11, 12, 13, 14, 15, 16, 17, 18, 19, 20, 23, 24, 25, 26, and fragments thereof; 3) a polypeptide consisting essentially of the amino acid sequence of SEQ ID NO: 1, 3, 5, 7, 8, 9, 10, 11, 12, 13, 14, 15, 16, 17, 18, 19, 20, 23, 24, 25, 26, or fragment thereof; 4) a polypeptide consisting of the amino acid sequence of SEQ ID NO: 1, 3, 5, 7, 8, 9, 10, 11, 12, 13, 14, 15, 16, 17, 18, 19, 20, 23, 24, 25, 26, or fragment thereof; 5) a nucleic acid sequence having at least 70% (e.g. 70%, 75%, 80%, 85%, 90%, 91%, 92%, 93%, 94%, 95%, 96%, 97%, 98%, 99% or 100%) sequence identity with a nucleic acid sequence encoding any one of the peptides listed above; 6) a nucleic acid sequence having at least 70% to 99% (e.g., 70%, 75%, 80%, 85%, 90%, 91%, 92%, 93%, 94%, 95%, 96%, 97%, 98% or 99%) sequence identity to a nucleic acid sequence encoding the listed polypeptides; 7) a nucleic acid sequence consisting essentially of the nucleic acid sequence described above; and 8) a nucleic acid sequence consisting of the nucleic acid sequence described above. A fragment of these polypeptides can be approximately 8-56 amino acid residues, such as 8, 9, 10, 20, 30, 40, 50, 51, 52, 53, 54, 55, and 56 residues.

The vaccine composition can further comprise an adjuvant and/or a carrier. Examples of useful adjuvants and carriers are given herein below. The peptides and/or polypeptides in the composition can be associated with a carrier such as e.g. a protein or an antigen-presenting cell such as e.g. a dendritic cell (DC) capable of presenting the peptide to a T-cell.

Adjuvants are any substance whose admixture into the vaccine composition increases or otherwise modifies the immune response to the mutant peptide. Carriers are scaffold structures, for example a polypeptide or a polysaccharide, to which the neoantigenic peptides, are capable of being associated. Optionally, adjuvants are conjugated covalently or non-covalently to the peptides or polypeptides of the invention.

The ability of an adjuvant to increase the immune response to an antigen is typically manifested by a significant increase in immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th response into a primarily cellular, or Th response.

Suitable adjuvants include, but are not limited to aluminium salts, Montanide ISA 206, Montanide ISA 50V, Montanide ISA 50, Montanide ISA-51, Montanide ISA-720, 1018 ISS, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, Juvlmmune, LipoVac, MF59, monophosphoryl lipid A, Montanide IMS 1312, OK-432, OM-174, OM-197-MP-EC, ONTAK, PepTel® vector system, PLG microparticles, resiquimod, SRL172, Virosomes and other Virus-like particles, YF-17D, VEGF trap, R848, beta-glucan, Pam3Cys, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Adjuvants such as incomplete Freund's or GM-CSF are preferred. Several immunological adjuvants (e.g., MF59) specific for dendritic cells and their preparation have been described previously (Dupuis M, et al., Cell Immunol. 1998; 186(1):18-27; Allison A C; Dev Biol Stand. 1998; 92:3-11). Also cytokines may be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12) (Gabrilovich D I, et al., J Immunother Emphasis Tumor Immunol. 1996 (6):414-418).

Other examples of useful immunostimulatory agents include, but are not limited to, Toll-like Receptor (TLR) agonists such as chemically modified CpGs (e.g. CpR, Idera), Poly(I:C)(e.g. polyi:CI2U), non-CpG bacterial DNA or RNA as well as immunoactive small molecules, such as cyclophosphamide, which may act therapeutically and/or as an adjuvant. The amounts and concentrations of adjuvants and additives useful in the context of the present invention can readily be determined by the skilled artisan without undue experimentation. Additional adjuvants include colony-stimulating factors, such as Granulocyte Macrophage Colony Stimulating Factor (GM-CSF, sargramostim). The vaccine may also contain a blocker of PD-L1 (CD274) binding to its receptor (PD-1) or to CD80 to prevent/inhibit the development of T regulatory cells (Treg) and thereby reducing the development of tolerance to the vaccine antigen. An exemplary PD-1 inhibitor is Bristol Meyers Squibb's BMS-936558 (also known as MDX-1106 and ONO-4538).

A vaccine composition according to the invention may comprise more than one different adjuvant. Furthermore, the invention encompasses a therapeutic composition comprising any adjuvant substance including any of the above or combinations thereof. It is also contemplated that the peptide or polypeptide, and the adjuvant can be administered separately in any appropriate sequence.

A carrier may be present independently of an adjuvant. The function of a carrier can for example be to increase the molecular weight of a particular mutant in order to increase their activity or immunogenicity, to confer stability, to increase the biological activity, or to increase serum half-life. Furthermore, a carrier may aid presenting peptides to T-cells. The carrier may be any suitable carrier known to the person skilled in the art, for example a protein or an antigen presenting cell. A carrier protein could be but is not limited to keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier must be a physiologically acceptable carrier acceptable to humans and safe. However, tetanus toxoid and/or diphtheria toxoid are suitable carriers in one embodiment of the invention. Alternatively, the carrier may be dextrans for example sepharose.

Cytotoxic T-cells (CTLs) recognize an antigen in the form of a peptide bound to an MHC molecule rather than the intact foreign antigen itself. The MHC molecule itself is located at the cell surface of an antigen presenting cell. Thus, an activation of CTLs is only possible if a trimeric complex of peptide antigen, MHC molecule, and antigen-presenting cell (APC) is present. Correspondingly, it may enhance the immune response if not only the peptide is used for activation of CTLs, but if additionally APCs with the respective MHC molecule are added. Therefore, in some embodiments the vaccine composition according to the present invention additionally contains at least one antigen presenting cell.

In the case of a DNA vaccine, a nucleic acid comprising the sequence of SEQ ID NOs: 2, 4, 6 or fragment thereof is formulated in a eukaryotic vector for use as a vaccine that is administered to human subjects. The nucleotides encoding the antigen are operably linked promoter and other regulatory sequences in the vector. Such eukaryotic, e.g., mammalian vectors, are known in the art [e.g., pcDNA™ (Invitrogen) and vectors available from Vical Inc. (San Diego, Calif.)]. Other exemplary vectors, e.g., pNGVL4a, and derivatives thereof, are described in Moorty et al., 2003, Vaccine 21:1995-2002; Cebere et al., 2006, Vaccine 24:41-425; or Trimble et al., 2009, Clin. Cancer Res. 15:364-367; hereby incorporated by reference).

### Recombinant Expression Vectors and Host Cells

The antigens described herein can be made by any recombinant method that provides the epitope of interest. Accordingly, another aspect of the invention pertains to vectors, preferably expression vectors, containing a nucleic acid encoding any polypeptides described herein, or derivatives, fragments, analogs or homologs thereof (e.g., SEQ ID Nos: 1, 3, 5, 7, 8, 9, 10, 11, 12, 13, 14, 15, 16, 17, 18, 19, and 20). As used herein, the term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Additionally, some viral vectors are capable of targeting a particular cell type either specifically or non-specifically.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, “operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue specific regulatory sequences). It will be appreciated by those skilled in the art that the design of the expression vector can depend on such factors as the choice of the host cell to be transformed, the level of expression of protein desired. The expression vectors of the invention can be introduced into host cells to thereby produce proteins or peptides, including fusion proteins or peptides, encoded by nucleic acids as described herein (e.g., Frag1, Frag2, C-Term, mutant forms of Frag1, Frag2, C-Term, fusion proteins, etc.).

The recombinant expression vectors of the invention can be designed for expression of any of the polypeptides or polynucleotide sequences of the present invention in prokaryotic or eukaryotic cells. For example, any of the polypeptides or polynucleotide sequences of the present invention can be expressed in bacterial cells such as E. coli, insect cells (using baculovirus expression vectors), yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31 40), pMAL (New England Biolabs, Beverly, Mass.) and pRIT5 (Pharmacia, Piscataway, N.J.) that fuse glutathione S transferase (GST), maltose E binding protein, or protein A, respectively, to the target recombinant protein. Examples of suitable inducible non fusion E. coli expression vectors include pTrc (Amrann et al., (1988) Gene 69:301 315) and pET11d (Studier et al., GENE EXPRESSION TECHNOLOGY: METHODS IN ENZYMOLOGY 185, Academic Press, San Diego, Calif. (1990) 60 89).

One strategy to maximize recombinant protein expression in E. coli is to express the protein in host bacteria with an impaired capacity to proteolytically cleave the recombinant protein. See, Gottesman, GENE EXPRESSION TECHNOLOGY: METHODS IN ENZYMOLOGY 185, Academic Press, San Diego, Calif. (1990) 119 128. Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111 2118). Such alteration of nucleic acid sequences of the invention can be carried out by standard DNA synthesis techniques. We used ATUM's (formally DNA2.0, www.atum.bio) method to codon optimize the nucleotide sequences for rPfEMMA1 and rPbEMMA1 (See SEQ ID NOs: 27 and 28).

In another embodiment, the expression vector is a yeast expression vector. Examples of vectors for expression in yeast S. cerevisiae include pYepSec1 (Baldari, et al., (1987) EMBO J 6:229 234), pMFa (Kurjan and Herskowitz, (1982) Cell 30:933 943), pJRY88 (Schultz et al., (1987) Gene 54:113 123), pYES2 (Invitrogen Corporation, San Diego, Calif.), and picZ (InVitrogen Corp, San Diego, Calif.).

Alternatively, any of the polypeptides or polynucleotide sequences described herein can be expressed in insect cells using baculovirus expression vectors. Baculovirus vectors available for expression of proteins in cultured insect cells (e.g., SF9 cells) include the pAc series (Smith et al., (1983) Mol Cell Biol 3:2156 2165) and the pVL series (Lucklow and Summers (1989) Virology 170:31 39). Another commonly used cell line for malaria antigen expression is the Schneider 2 (S2) cell line derived from the insect, Drosophila melanogaster.

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al., (1987) EMBO J 6: 187 195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see, e.g., Chapters 16 and 17 of Sambrook et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue specific regulatory elements are used to express the nucleic acid). Tissue specific regulatory elements are known in the art. Non limiting examples of suitable tissue specific promoters include the albumin promoter (liver specific; Pinkert et al., (1987) Genes Dev 1:268 277), lymphoid specific promoters (Calame and Eaton (1988) Adv Immunol 43:235 275), in particular promoters of T cell receptors (Winoto and Baltimore (1989) EMBO J 8:729 733) and immunoglobulins (Banerji et al., (1983) Cell 33:729 740; Queen and Baltimore (1983) Cell 33:741 748), neuron specific promoters (e.g., the neurofilament promoter; Byrne and Ruddle (1989) PNAS 86:5473 5477), pancreas specific promoters (Edlund et al., (1985) Science 230:912 916), and mammary gland specific promoters (e.g., milk whey promoter; U.S. Pat. No. 4,873,316 and European Application Publication No. 264,166). Developmentally regulated promoters are also encompassed, e.g., the murine hox promoters (Kessel and Gruss (1990) Science 249:374 379) and the α-fetoprotein promoter (Campes and Tilghman (1989) Genes Dev 3:537 546).

The invention further provides a recombinant expression vector comprising a DNA molecule of the invention cloned into the expression vector in an antisense orientation. That is, the DNA molecule is operatively linked to a regulatory sequence in a manner that allows for expression (by transcription of the DNA molecule) of an RNA molecule that is antisense to messenger RNA (mRNA) of any of the polynucleotide sequences of the present invention. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al., “Antisense RNA as a molecular tool for genetic analysis,” Reviews Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, any of the polypeptides or polynucleotide sequences of the present invention can be expressed in bacterial cells such as E. coli, insect cells, yeast or mammalian cells (such as Chinese hamster ovary cells (CHO) or CV-1 in Origin with SV40 genes (COS) cells). Alternatively, a host cell can be a premature mammalian cell, i.e., pluripotent stem cell. A host cell can also be derived from other human tissue. Other suitable host cells are known to those skilled in the art.

Vector DNA can be introduced into prokaryotic or eukaryotic cells via conventional transformation, transduction, infection or transfection techniques. As used herein, the terms “transformation” “transduction”, “infection” and “transfection” are intended to refer to a variety of art recognized techniques for introducing foreign nucleic acid (e.g., DNA) into a host cell, including calcium phosphate or calcium chloride co precipitation, DEAE dextran mediated transfection, lipofection, or electroporation. In addition transfection can be mediated by a transfection agent. By “transfection agent” is meant to include any compound that mediates incorporation of DNA in the host cell, e.g., liposome. Suitable methods for transforming or transfecting host cells can be found in Sambrook, et al., (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals. Transfection may be “stable” (i.e. integration of the foreign DNA into the host genome) or “transient” (i.e., DNA is episomally expressed in the host cells).

For stable transfection of mammalian cells, it is known that, depending upon the expression vector and transfection technique used, only a small fraction of cells may integrate the foreign DNA into their genome the remainder of the DNA remains episomal. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding any of the polypeptides or polynucleotide sequences of the present invention or it can be introduced on a separate vector. Cells stably transfected with the introduced nucleic acid can be identified by drug selection (e.g., cells that have incorporated the selectable marker gene will survive, while the other cells die). In a specific embodiment, the promoter is the insulin promoter driving the expression of green fluorescent protein (GFP).

In one embodiment nucleic acid of any of the polypeptides or polynucleotide sequences described herein is present in a viral vector. In another embodiment, the nucleic acid is encapsulated in a virus. In some embodiments, the virus preferably infects pluripotent cells of various tissue types, e.g. hematopoietic stem cells, neuronal stem cells, hepatic stem cells or embryonic stem cells, preferably the virus is hepatropic. By “hepatotropic” it is meant that the virus has the capacity to preferably target the cells of the liver either specifically or non-specifically. In further embodiments the virus is a modulated hepatitis virus, SV-40, or Epstein-Bar virus. In yet another embodiment, the virus is an adenovirus.

“Recombinant” refers to an artificial combination of two otherwise separated segments of sequence, e.g., by chemical synthesis or by the manipulation of isolated segments of nucleic acids by genetic engineering techniques.

A transgenic mammal can also be used in order to express the protein of interest encoded by one or both of the above-described nucleic acid sequences. More specifically, once the above-described construct is created, it can be inserted into the pronucleus of an embryo. The embryo can then be implanted into a recipient female. Alternatively, a nuclear transfer method could also be utilized (Schnieke et al., 1997). Gestation and birth are then permitted to occur (see, e.g., U.S. Pat. Nos. 5,750,176 and 5,700,671), and milk, tissue or other fluid samples from the offspring should then contain the protein of interest. The mammal utilized as the host can be selected from the group consisting of, for example, a mouse, a rat, a rabbit, a pig, a goat, a sheep, a horse and a cow. However, any mammal can be used provided it has the ability to incorporate DNA encoding the protein of interest into its genome.

### Antibodies

“Antibody” (Ab) comprises single Abs directed against a target antigen (an anti-target antigen Ab), anti-target antigen Ab compositions with poly-epitope specificity, single chain anti-target antigen Abs, and fragments of anti-target antigen Abs. A “monoclonal antibody” (mAb) is obtained from a population of substantially homogeneous Abs, i.e., the individual Abs comprising the population are identical except for possible naturally-occurring mutations that can be present in minor amounts. Exemplary Abs include polyclonal (pAb), monoclonal (mAb), humanized, bi-specific (bsAb), and heteroconjugate Abs. The invention encompasses not only an intact monoclonal antibody, but also an immunologically-active antibody fragment, e. g., a Fab or (Fab)2 fragment; an engineered single chain Fv molecule; or a chimeric molecule, e.g., an antibody which contains the binding specificity of one antibody, e.g., of murine origin, and the remaining portions of another antibody, e.g., of human origin.

Also provided herein are antibodies to the following antigens (as a vaccination): 1) a polypeptide having at least 70% (e.g. 70%, 75%, 80%, 85%, 90%, 91%, 92%, 93%, 94%, 95%, 96%, 97%, 98%, 99% or 100%) sequence identity with an amino acid sequence selected from the group consisting of SEQ ID NOs: 1, 3, 5, 7, 8, 9, 10, 11, 12, 13, 14, 15, 16, 17, 18, 19, 20, 23, 24, 25, 26, and fragments thereof; 2) a polypeptide having at least 70% to 99% (e.g., 70%, 75%, 80%, 85%, 90%, 91%, 92%, 93%, 94%, 95%, 96%, 97%, 98% or 99%) sequence identity to an amino acid sequence selected from the group consisting of SEQ ID NOs: 1, 3, 5, 7, 8, 9, 10, 11, 12, 13, 14, 15, 16, 17, 18, 19, 20, 23, 24, 25, 26, and fragment thereof; 3) a polypeptide consisting essentially of the amino acid sequence of SEQ ID NOs: 1, 3, 5, 7, 8, 9, 10, 11, 12, 13, 14, 15, 16, 17, 18, 19, 20, 23, 24, 25, 26, or fragment thereof; 4) a polypeptide consisting of the amino acid sequence of SEQ ID NOs: 1, 3, 5, 7, 8, 9, 10, 11, 12, 13, 14, 15, 16, 17, 18, 19, 20, 23, 24, 25, 26, or fragment thereof. A fragment of these polypeptides can be approximately 8-56 amino acid residues, such as 8, 9, 10, 20, 30, 40, 50, 51, 52, 53, 54, 55, and 56 residues.

Polyclonal Abs can be raised in a mammalian host by one or more injections of an immunogen and, if desired, an adjuvant. Monoclonal antibodies of the invention can be produced by any hybridoma liable to be formed according to classical methods from splenic or lymph node cells of an animal, particularly from a mouse or rat, immunized against the polypeptides or peptides according to the invention.

The antigen and antibody of the present invention can be attached to a signal generating compound or “label”. This signal generating compound or label is in itself detectable or can be reacted with one or more additional compounds to generate a detectable product. Examples of such signal generating compounds include chromogens, radioisotopes (e.g., ¹²⁵I, ¹³¹I, ³²P, ³²H, ³⁵S, and ¹⁴C), fluorescent compounds (e.g., fluorescein, rhodamine), chemiluminescent compounds, particles (visible or fluorescent), nucleic acids, complexing agents, or catalysts such as enzymes (e.g., alkaline phosphatase, acid phosphatase, horseradish peroxidase, β-galactosidase, and ribonuclease). In the case of enzyme use, addition of chromo-, fluoro-, or lumo-genic substrate results in generation of a detectable signal. Other detection systems such as time-resolved fluorescence, internal-reflection fluorescence, amplification (e.g., polymerase chain reaction) and Raman spectroscopy are also useful.

### Therapeutic Methods

The invention further provides a method of inducing a P. falciparum specific immune response in a subject, vaccinating against malaria, reducing the need for treatment and/or alleviating a symptom of malaria in a subject by administering the subject a peptide or vaccine composition of the invention.

The subject has been diagnosed with malaria or is at risk of developing malaria. The subject has resistant malaria. The subject is a human, dog, cat, horse or any animal in which a P. falciparum specific immune response is desired. The subject is a pregnant female. Preferably, the subject is a child under 5 years old of age. More preferably, the subject is an infant, e.g., at least about 6-8 weeks old of age.

The peptide or composition of the invention is administered in an amount sufficient to induce an immune response.

The invention provides methods of treating, reducing the severity of or preventing malaria by administering to a subject one or more peptides of the instant invention. The antigen peptide, polypeptide, nucleic acid sequences or vaccine composition of the invention can be administered alone or in combination with one or more therapeutic agents. The therapeutic agent is, for example, one, two, three, four, or more additional vaccines, antimalarials such as artemisinin-combination therapy, or an immunotherapy. Any suitable therapeutic treatment for malaria may be administered. The additional vaccine may comprise an inhibitor of parasite liver invasion or an inhibitor of parasite RBC invasion. Such additional vaccines include, but are not limited to, anti-RBC invasion vaccines (PfMSP-1, PfRH5), RTS,S (Mosquirix), NYVAC-Pf7, PfCSP, and [NANP]19-5.1. The antigen peptide, polypeptide, nucleic acid sequences, or vaccine composition of the invention can be administered prior to, concurrently, or after other therapeutic agents.

A suitable amount of each peptide to be included in the vaccine composition and the optimum dosing regimen can be determined by one skilled in the art without undue experimentation. For example, the peptide or its variant may be prepared for intravenous (i.v.) injection, subcutaneous (s.c.) injection, intradermal (i.d.) injection, intraperitoneal (i.p.) injection, intramuscular (i.m.) injection. Preferred methods of peptide injection include s.c., i.d., i.p., i.m., and i.v. Preferred methods of DNA injection include i.d., i.m., s.c., i.p. and i.v. For example, doses of between 1 and 500 mg, 50 μg and 1.5 mg, preferably 125 μg to 500 μg, of peptide or DNA may be given and will depend on the respective peptide or DNA. Doses of this range were successfully used in previous trials (Brunsvig P F, et al., Cancer Immunol Immunother. 2006; 55(12):1553-1564; M. Staehler, et al., ASCO meeting 2007; Abstract No 3017). Other methods of administration of the vaccine composition are known to those skilled in the art.

Pharmaceutical compositions comprising the peptide of the invention may be administered to an individual already suffering from malaria. In therapeutic applications, compositions are administered to a patient in an amount sufficient to elicit an effective immune response to the present antigen and to cure or at least partially arrest symptoms and/or complications. An amount adequate to accomplish this is defined as “therapeutically effective dose.” Amounts effective for this use will depend on, e.g., the peptide composition, the manner of administration, the stage and severity of the disease being treated, the weight and general state of health of the patient, and the judgment of the prescribing physician, but generally range for the initial immunization (that is for therapeutic or prophylactic administration) from about 1.0 μg to about 50,000 μg of peptide for a 70 kg patient, followed by boosting dosages or from about 1.0 μg to about 10,000 μg of peptide pursuant to a boosting regimen over weeks to months depending upon the patient's response and condition by measuring specific immune activity in the patient's blood.

The pharmaceutical compositions (e.g., vaccine compositions) for therapeutic treatment are intended for parenteral, topical, nasal, oral or local administration. Preferably, the pharmaceutical compositions are administered parenterally, e.g., intravenously, subcutaneously, intradermally, or intramuscularly. Preferably, the vaccine is administered intramuscularly. The invention provides compositions for parenteral administration which comprise a solution of the peptides and vaccine compositions are dissolved or suspended in an acceptable carrier, preferably an aqueous carrier. A variety of aqueous carriers may be used, e.g., water, buffered water, 0.9% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

The concentration of peptides of the invention in the pharmaceutical formulations can vary widely, i.e., from less than about 0.1%, usually at or at least about 2% to as much as 20% to 50% or more by weight, and will be selected primarily by fluid volumes, viscosities, etc., in accordance with the particular mode of administration selected.

The peptide of the invention may also be administered via liposomes, which target the peptides to a particular tissue, such as lymphoid tissue. Liposomes are also useful in increasing the half-life of the peptides. Liposomes include emulsions, foams, micelles, insoluble monolayers, liquid crystals, phospholipid dispersions, lamellar layers and the like. In these preparations, the peptide to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes filled with a desired peptide of the invention can be directed to the site of lymphoid cells, where the liposomes then deliver the selected therapeutic/immunogenic peptide compositions. Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al., Ann. Rev. Biophys. Bioeng. 9; 467 (1980), USAU.S. Pat. Nos. 4,235,871, 4,501,728 4,501,728, 4,837,028, and 5,019,369.

For targeting to the immune cells, a ligand to be incorporated into the liposome can include, e.g., antibodies or fragments thereof specific for cell surface determinants of the desired immune system cells. A liposome suspension containing a peptide may be administered intravenously, locally, topically, etc. in a dose which varies according to, inter alia, the manner of administration, the peptide being delivered, and the stage of the disease being treated.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more peptides of the invention, and more preferably at a concentration of 25%-75%.

For aerosol administration, the immunogenic peptides are preferably supplied in finely divided form along with a surfactant and propellant. Typical percentages of peptides are 0.01%-20% by weight, preferably 1%-10%. The surfactant must be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included as desired, as with, e.g., lecithin for intranasal delivery.

For therapeutic or immunization purposes, nucleic acids encoding the peptide of the invention and optionally one or more of the peptides described herein can also be administered to the patient. A number of methods are conveniently used to deliver the nucleic acids to the patient. For instance, the nucleic acid can be delivered directly, as “naked DNA”. This approach is described, for instance, in Wolff et al., Science 247: 1465-1468 (1990) as well as USA U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles.

The nucleic acids can also be delivered complexed to cationic compounds, such as cationic lipids. Lipid-mediated gene delivery methods are described, for instance, in 9618372WOAWO 96/18372; 9324640WOAWO 93/24640; Mannino & Gould-Fogerite, BioTechniques 6(7): 682-691 (1988); U.S. Pat. No. 5,279,833 Rose U.S. Pat. Nos. 5,279,833; 9,106,309WOAWO 91/06309; and Felgner et al., Proc. Natl. Acad. Sci. USA 84: 7413-7414 (1987).

The peptides and polypeptides of the invention can also be expressed by attenuated viral hosts, such as vaccinia or fowlpox. This approach involves the use of vaccinia virus as a vector to express nucleotide sequences that encode the peptide of the invention. Upon introduction into an acutely or chronically infected host or into a noninfected host, the recombinant vaccinia virus expresses the immunogenic peptide, and thereby elicits a host CTL response. Vaccinia vectors and methods useful in immunization protocols are described in, e.g., U.S. Pat. No. 4,722,848. Another vector is BCG (Bacille Calmette Guerin). BCG vectors are described in Stover et al., (Nature 351:456-460 (1991)). A wide variety of other vectors useful for therapeutic administration or immunization of the peptides of the invention, e.g., Salmonella typhi vectors and the like, will be apparent to those skilled in the art from the description herein.

A preferred means of administering nucleic acids encoding the peptide of the invention uses minigene constructs encoding multiple epitopes. To create a DNA sequence encoding the selected CTL epitopes (minigene) for expression in human cells, the amino acid sequences of the epitopes are reverse translated. A human codon usage table is used to guide the codon choice for each amino acid. These epitope-encoding DNA sequences are directly adjoined, creating a continuous polypeptide sequence. To optimize expression and/or immunogenicity, additional elements can be incorporated into the minigene design. Examples of amino acid sequence that could be reverse translated and included in the minigene sequence include: helper T lymphocyte, epitopes, a leader (signal) sequence, and an endoplasmic reticulum retention signal. In addition, major histocompatibility complex (MHC) presentation of CTL epitopes may be improved by including synthetic (e.g. poly-alanine) or naturally-occurring flanking sequences adjacent to the CTL epitopes.

The dosing regimen that can be used in the methods of the invention includes, but is not limited to, daily, three times weekly (intermittent), two times weekly, weekly, or every 14 days. Alternatively, dosing regimen includes, but is not limited to, monthly dosing or dosing every 6-8 weeks. The vaccine of the present invention can be administered intramuscularly once every two weeks for 1, 2, 3, 4, 5, or more times, alone or in combination with 1, 2, 3, 4, or more additional vaccines in a subject, preferably a human subject.

Also provided herein is a method of treating P. falciparum malaria in a subject in need of by administering a therapeutically effective amount of an antibody described herewith to the subject. Preferably, the antibody is a purified monoclonal antibody, e.g., one that has been raised to and is specific for the protein described herein (e.g., Frag1, Frag2, C-Term). For example, the monoclonal antibody is a humanized antibody. The treatment can be initiated at an early stage after the appearance of recrudescent parasites. The symptoms of the subject may be mild or absent and parasitemia is low but increasing, for example from range 4,000-10,000/ul. Alternative, the subject may have fever <38.5° C. without any other accompanying symptom. The subject can be a child under 10 years of age. The subject can also be an elder child or an adult. In one example, the subject is characterized as suffering from acute P. falciparum malaria but has not responded to treatment with anti-malarial drugs. In this passive immunity approach, the purified humanized monoclonal antibody that binds specifically to the protein described herein, preferably SEQ ID NOs: 1, 3, and 5, is administered to the subject to kill the infective agent and/or inhibit RBC invasion.

The antibody can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form. Preferably, the antibody is administered intravenously or intramuscularly. For example, the antibody is administered in 1-2 gram amounts, 1, 2, 3, or 4 times. The dosing regimen that can be used in the methods of the invention includes, but is not limited to, daily, three times weekly (intermittent), two times weekly, weekly, or every 14 days. Alternatively, dosing regimen includes, but is not limited to, monthly dosing or dosing every 6-8 weeks. The antibody of the present invention can be administered intravenously once, twice or three times alone or in combination with 1, 2, 3, 4, or more additional therapeutic agents in a subject, preferably a human subject. The additional therapeutic agent is, for example, one, two, three, four, or more additional vaccines or antibodies, antimalarials such as artemisinin-combination therapy, or an immunotherapy. Any suitable therapeutic treatment for malaria may be administered. The additional vaccine may comprise an inhibitor of parasite liver invasion or an inhibitor of parasite RBC invasion. Such additional vaccines include, but are not limited to, anti-RBC invasion vaccines (PfMSP-1, PfRH5), RTS,S (Mosquirix), NYVAC-Pf7, PfCSP, and [NANP]19-5.1. The antibody of the invention can be administered prior to, concurrently, or after other therapeutic agents.

Amounts effective for this use will depend on, e.g., the antibody composition, the manner of administration, the stage and severity of P. falciparum malaria being treated, the weight and general state of health of the patient, and the judgment of the prescribing physician, but generally range for the treatment from about 10 mg/kg (weight of a subject) to 300 mg/kg, preferably 20 mg/kg-200 mg/kg.

### Kits

Kits are also included within the scope of the present invention. The present invention includes kits for determining the presence of antibodies to P. falciparum in a test sample. A kit can comprise: 1) an antigen comprising a polypeptide having at least 70% (e.g. 70%, 75%, 80%, 85%, 90%, 91%, 92%, 93%, 94%, 95%, 96%, 97%, 98%, 99% or 100%) sequence identity with an amino acid sequence selected from the group consisting of SEQ ID NOs: 1, 3, 5, 7, 8, 9, 10, 11, 12, 13, 14, 15, 16, 17, 18, 19, 20, 23, 24, 25, 26, and fragment thereof; and (b) a conjugate comprising an antibody attached to a signal-generating compound capable of generating a detectable signal. The kit can also contain a control or calibrator which comprises a reagent which binds to the antigen. The antigen can comprise a polypeptide consisting essentially of the amino acid sequences of SEQ ID NO: 1, 3, 5, 7, 8, 9, 10, 11, 12, 13, 14, 15, 16, 17, 18, 19, 20, 23, 24, 25, 26, or fragment thereof. Finally, the antigen can consist of the amino acid sequences of SEQ ID NO: 1, 3, 5, 7, 8, 9, 10, 11, 12, 13, 14, 15, 16, 17, 18, 19, 20, 23, 24, 25, 26, or fragment thereof. A fragment of these polypeptides can be approximately 8-56 amino acid residues, such as 8, 9, 10, 20, 30, 40, 50, 51, 52, 53, 54, 55, and 56 residues.

The invention described herein also provides a pharmaceutical pack or kit comprising one or more containers filled with the vaccine in a form suitable for intramuscular administration or other routes of administration. The kits described herein may also contain one or more antibodies described herewith. Optionally the kit may contain disposable items, such as biodegradable items. The kit may also contain a sample collection means, including, but not limited to a needle for collecting blood, storage means for storing the collected sample, and for shipment. Alternatively, any kits described herein may contain an instruction for use to diagnose malaria or a receptacle for receiving subject derived bodily fluid or tissue.

The kit further comprises instructions for use or a CD, or CD-ROM with instructions on how to collect sample, ship sample, and means to interpret test results. The kit may also contain a control sample either positive or negative or a standard and/or an algorithmic device for assessing the results and additional reagents and components.

A “biological sample” is any bodily fluid or tissue sample obtained from a subject, including, but is not limited to, blood, blood serum, urine, and saliva.

The kit may further comprise one or more additional compounds to generate a detectable product. Examples of such signal generating compounds include chromogens, radioisotopes (e.g., ¹²⁵I, ¹³¹I, ³²P, ³H, ³⁵S, and ¹⁴C), fluorescent compounds (e.g., fluorescein, rhodamine), chemiluminescent compounds, particles (visible or fluorescent), nucleic acids, complexing agents, or catalysts such as enzymes (e.g., alkaline phosphatase, acid phosphatase, horseradish peroxidase, β-galactosidase, and ribonuclease).

### EXAMPLES

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1: PfEMMA1 is a Highly Conserved, Low Polymorphic Parasite Protein

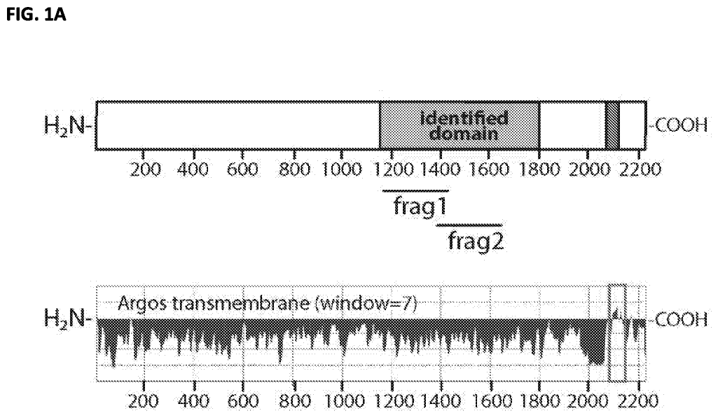

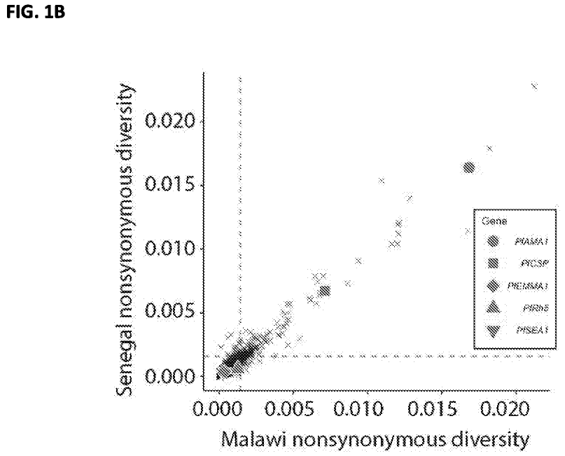

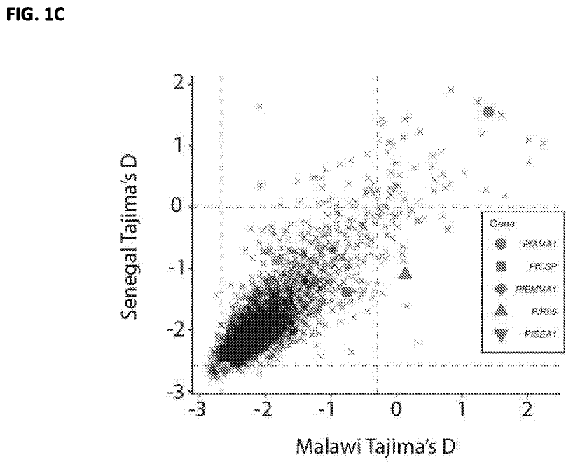

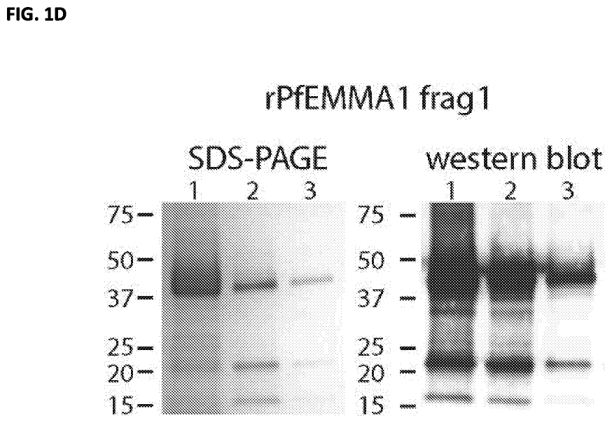

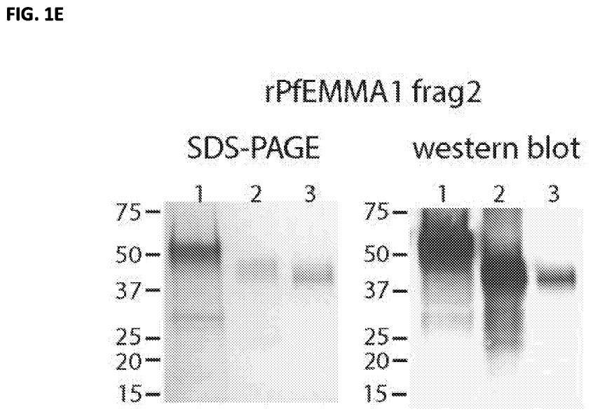

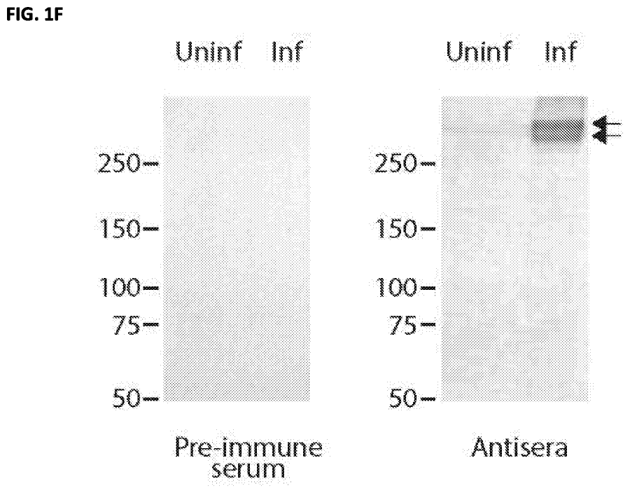

The P. falciparum 3D7 (Pf3D7) blood-stage proteome was screened with plasma from resistant and susceptible 2-year-old Tanzanian children and identified a segment of PF3D7_1134300 (nucleotide [nt] 3490-5412; amino acid [aa] 1164-1804) as a target of protective antibody responses associated with significantly lower parasitemia (D. K. Raj et al., Science 344, 871-877 (2014)). Bioinformatics analyses (PlasmoDB.org) predict a 263-kDa basic phosphoprotein with a single exon (M. Treeck, et al., Cell Host Microbe 10, 410-419 (2011)). Fifty percent of the protein comprises asparagine, glutamic acid, isoleucine and lysine, and approximately 25% of the protein contains low complexity regions (J. C. Wootton Comput Chem 18, 269-285 (1994)). Asparagine and glutamic acid constitute 73% of the six simple tandem repeats (aa 1995-2060), a feature that is common in exported proteins (C. Aurrecoechea et al., Nucleic Acids Res 37, D539-543 (2009) and A. Heiber et al., PLoS Pathog 9, e1003546 (2013)). It has been hypothesized that tandem repeats may elicit T-cell-independent antibody responses and mediate evasion of immunity by diverting effective responses away from critical epitopes (F. Verra, and A. L. Mol Biol Evol 16, 627-633 (1999)). A Protein BLAST search yielded no matches with an E-value <1 between PfEMMA1 and proteins of mammals or other genera suggesting this protein mediates one or more functions that are unique to malaria parasites (S. F. Altschul et al J Mol Biol 215, 403-410 (1990)). Functional analyses of the protein sequence did not classify PfEMMA1 in homologous superfamilies nor did they predict molecular functions or the presence of known domains (InterProScan v5.28-67.0, EMBL-EBI) (P. Jones et al., Bioinformatics 30, 1236-1240 (2014)). There is no predicted Plasmodium export element/host targeting signal (PEXEL/HT) domain, signal peptide or glycophosphatidylinositol (GPI) anchor (T. N. Petersen, et al., Nat Methods 8, 785-786 (2011), T. J. Sargeant et al., Genome Biol 7, R12 (2006), and A. Pierleoni, et al., BMC Bioinformatics 9, 392 (2008). However, there is a predicted transmembrane domain near the C-terminus (FIG. 1A) that corresponds to a hydrophobic region and α-helical structures (D. W. Buchan, et al., Nucleic Acids Res 41, W349-357 (2013)). PfEMMA1 expression increases during the second half of the asexual blood-stage life cycle according to transcriptional analyses reported in the Plasmodium genomics resource (PlasmoDB.org), N. Rovira-Graells et al., Genome Res 22, 925-938 (2012).

Pf gene sequences of single-clone lineages from samples collected in Senegal and Malawi were obtained from the MalariaGen Pf3k project, M. Manske et al., Nature 487,375,379(2012). Single nucleotide polymorphisms (SNPs) of PfEMMA1 were compared with those of PfRH5, PfSEA-1, PfCSP, and PfAMA1 after applying stringent quality-control filters. PfEMMA1 shows average levels of nonsynonymous nucleotide diversity (π_{NS}; 55^{th} percentile in Senegal, 52^{nd} percentile in Malawi), which contrasts with the high nucleotide diversity present in PfCSP and PfAMA1 (99th percentiles in Senegal and Malawi for each gene; FIG. 1B). In addition, the value of Tajima's D, a diversity-based statistic that can detect selection and/or demographic changes, is very low for PfEMMA1 in both populations (2^{nd} percentile in Senegal; 6^{th} percentile in Malawi; FIG. 1C). This indicates an excess of low frequency variants, a pattern suggesting that PfEMMA1 may be undergoing purifying selection. This selection appears uniform across geographic regions as population differentiation between West and East Africa (as measured by F_{ST}) is not elevated (62^{nd} percentile).

Using an expanded set of 1,315 globally distributed, single-clone infections from the Pf3k project, low polymorphism was found to be maintained outside Senegal and Malawi. Only one SNP in PfEMMA1 has a global minor allele frequency (MAF) greater than 0.05 (nt 294, MAF=0.118). Within the identified segment of PfEMMA1 (nt 3490-5412), no SNPs are at a frequency above 0.05 and only five have a combined MAF over 0.01 (N. Rovira-Graells et al., Genome Res 22, 925-938 (2012)). These data indicate that PfEMMA1 contains little amino acid variation on both local and global scales.

### Example 2: PfEMMA1 is Present in Maurer's Clefts and the RBC Membrane

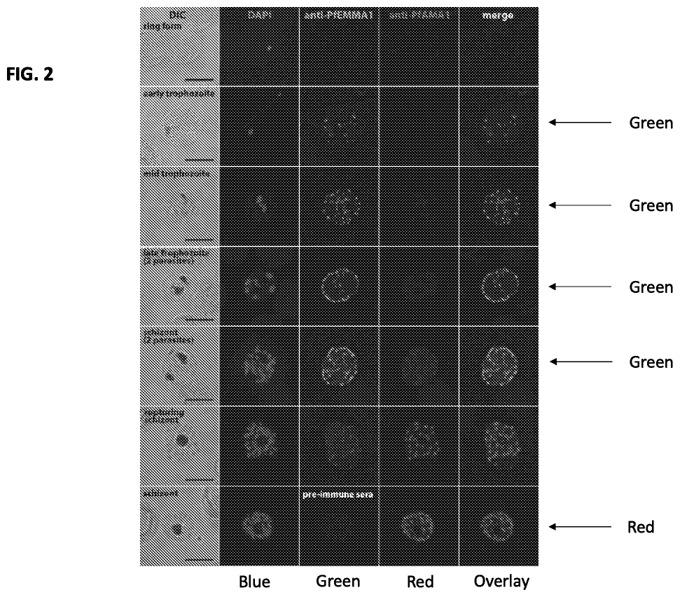

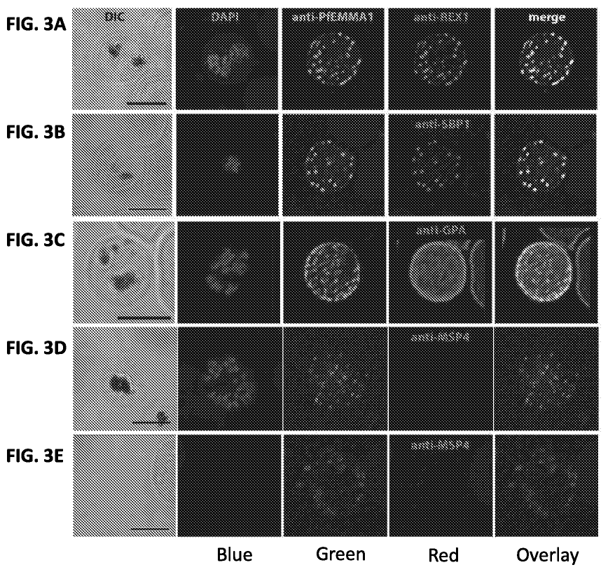

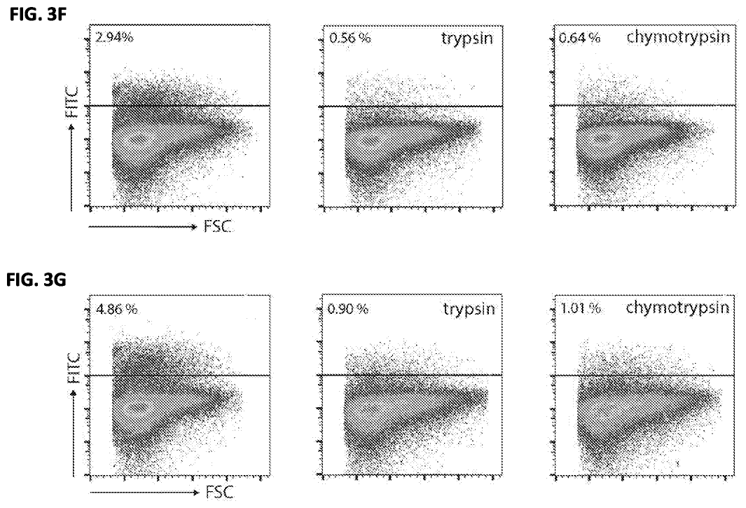

Antibodies to PfEMMA1 were generated by immunizing BALB/cJ mice with two codon-optimized, recombinantly expressed and purified polypeptides (fragments 1 and 2) derived from PfEMMA1 (FIG. 1A, D, E). PfEMMA1 encodes a native parasite protein; by probing lysates of mixed-stage Pf3D7-infected RBCs with murine anti-PfEMMA1 sera: two bands were detected by western blot. The band sizes (MW, 289 and 304 kDa; FIG. 1F) closely approximate the calculated MW (263 kDa) for unmodified full-length PfEMMA1. To determine the cellular localization of PfEMMA1, immunofluorescence confocal microscopy was performed in fixed and permeabilized RBCs infected with parasites of all stages using antibodies targeting PfEMMA1 fragments 1 and 2. The fluorescence pattern for PfEMMA1 reveals punctate structures beyond the parasite's plasma membrane in trophozoites, reminiscent of exported proteins (FIG. 2). Dual immunofluorescence labeling with ring-exported protein-1 (REX1; FIG. 3A) and skeleton-binding protein 1 (SBP1; FIG. 3B) show co-localization of PfEMMA1 with structural components of the Maurer's clefts (MC). Despite the lack of predicted canonical export signals, such as a signal peptide and PEXEL/HT motif, the localization of PfEMMA1 beyond the parasite membrane in addition to the protein having a predicted hydrophobic region categorizes it as a PEXEL-negative exported protein (PNEP) (A. Heiber et al., PLoS Pathog 9, e1003546 (2013)).

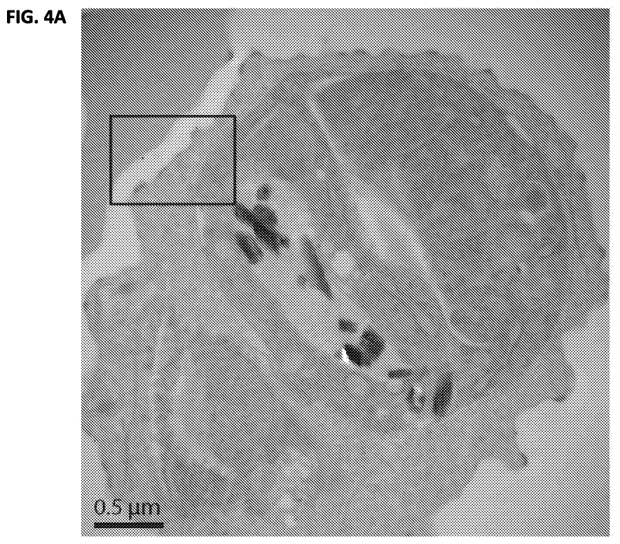

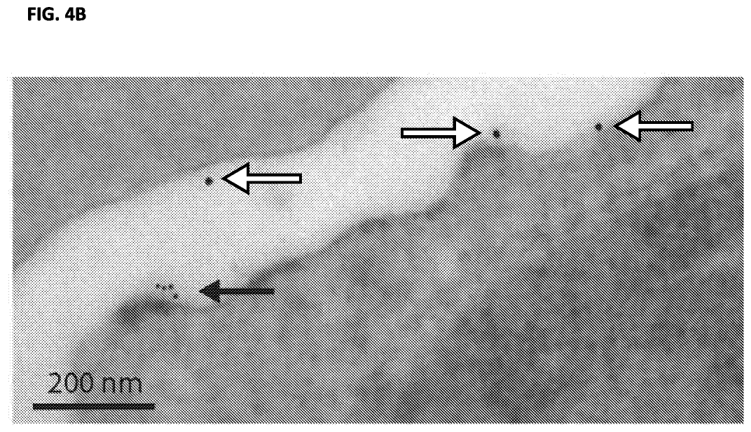

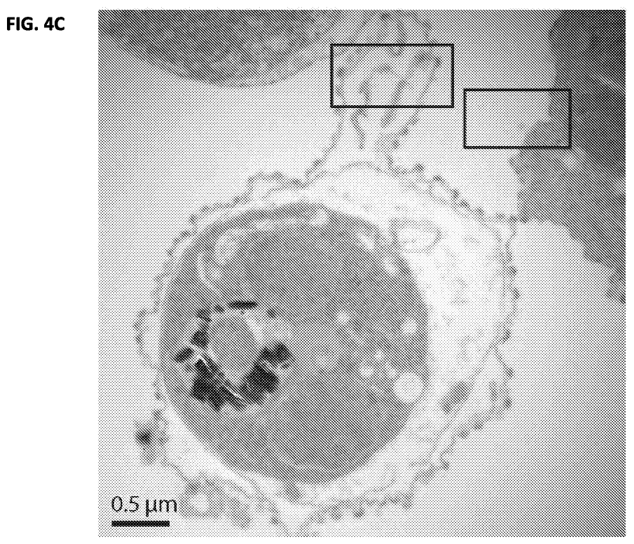

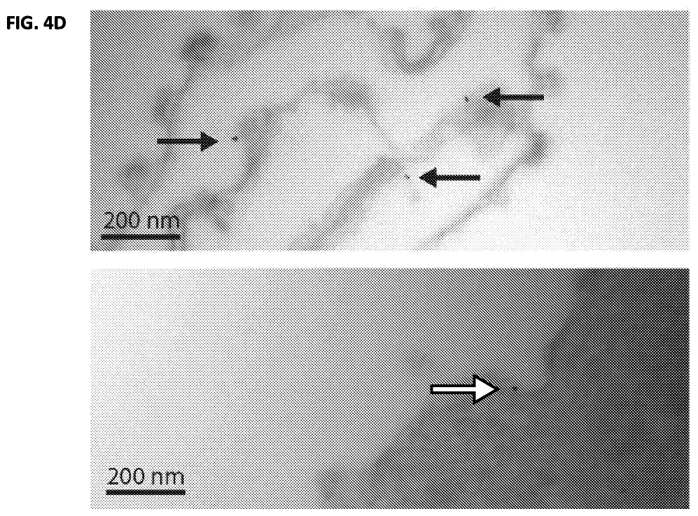

In permeabilized, late-stage trophozoites and schizonts, PfEMMA1 is localized to the periphery of host RBCs, and partially co-localizes with glycophorin A (GPA) detected using an anti-GPA antibody that specifically recognizes an intracellular GPA epitope (FIG. 3C). To determine whether PfEMMA1 is also exposed on the exofacial surface of the RBC plasma membrane, non-permeabilized, unfixed pRBCs were probed with anti-PfEMMA1 antibodies. A superficial stippled staining pattern was observed, indicating that PfEMMA1 is natively exposed on the surface of infected RBCs (FIG. 3D). Furthermore, PfEMMA1 was detected by immunofluorescence labeling of infected RBC remnant membranes of ghost cells devoid of parasite nuclei or RBC contents, indicating that it is integral to the disrupted membrane (FIG. 3E). The PfEMMA1 localization was confirmed on the exofacial surface of non-permeabilized host RBCs using flow cytometry. A small proportion (˜5%) of mixed late trophozoite- and schizont-infected cells were detected by fluorescently labeled anti-PfEMMA1 antibodies (Frag 1, FIG. 3F; Frag 2, FIG. 3G). To confirm surface localization, an identical sample was pretreated with trypsin and chymotrypsin to digest RBC surface proteins prior to surface immunolabeling with anti-PfEMMA1 antibodies. By comparing mean fluorescence intensities (MFI), it was determined that the anti-PfEMMA1 immunofluorescence signal decreased by ˜80% following protease treatment, indicating that at least a portion of PfEMMA1 is surface exposed and sensitive to trypsin and chymotrypsin (FIG. 3F, FIG. 3G). The immunofluorescence confocal microscopy and flow cytometry localization data were further corroborated by immuno-transmission electron microscopy studies in which it was observed that PfEMMA1 is distributed on the exofacial RBC surface on electron-dense knobs under native conditions (FIG. 4A, B), on the inner RBC leaflet, and in MCs after RBC permeabilization with equinatoxin II (EqtII; FIG. 4C, FIG. 4D) (K. E. Jackson et al., Biochem J 403, 167-175 (2007)).

### Example 3: PfEMMA1 is Expressed on the Merozoite Surface

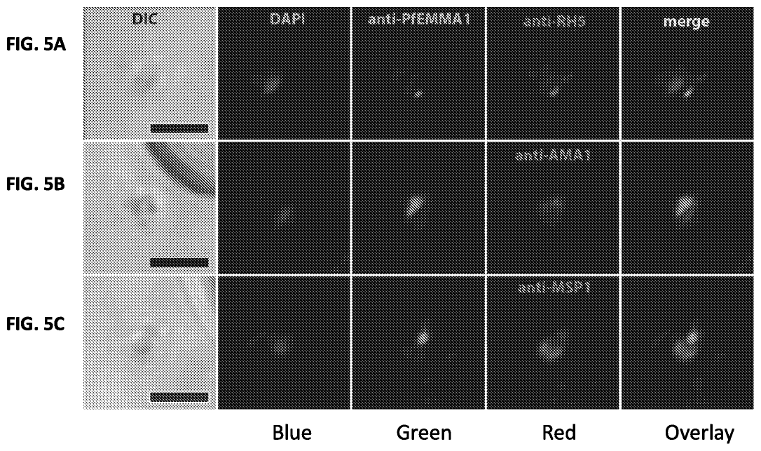

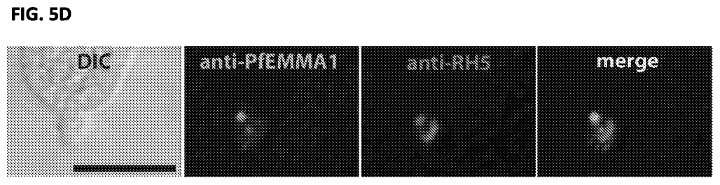

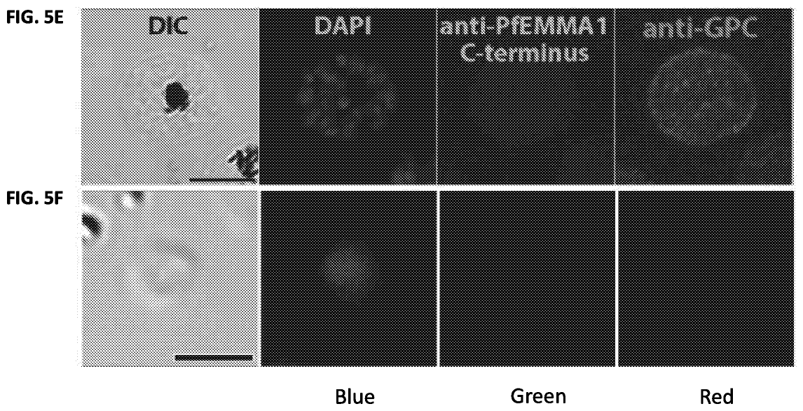

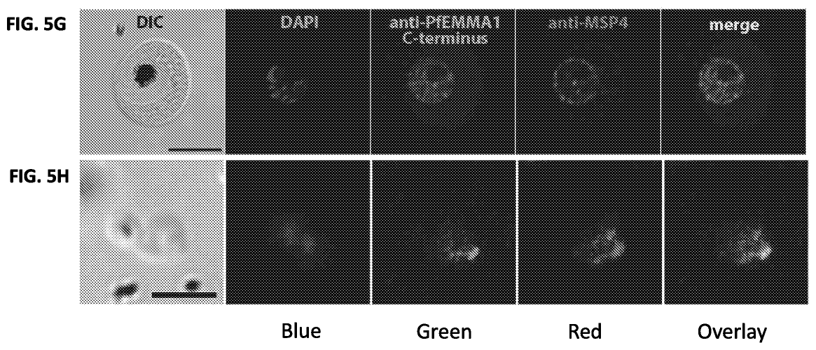

Using immunofluorescence confocal microscopy and antibodies that label fragment 2 of PfEMMA1, it was shown that the protein is displayed on the extrafacial surface of non-permeabilized merozoites (FIG. 5A-C). The protein is highly expressed at the apex of merozoites as demonstrated in a permeabilized merozoite in close contact with an uninfected RBC (FIG. 5D). PfEMMA1 is in close approximation to PfRH5.

### Example 4: PfEMMA1 is Oriented with an Interior C-Terminus

Polyclonal antibodies raised against the entire C-terminus region adjacent to the predicted transmembrane domain (FIG. 1A) failed to detect PfEMMA1 on the external surface of non-permeabilized pRBCs and merozoites (FIG. 5E, F), whereas the same antibodies labeled the protein after permeabilization of pRBCs (FIG. 5G, H). These findings lead us to hypothesize that some of the full length PfEMMA1 likely undergoes catalytic cleavage at or near its predicted transmembrane domain before it is exported to the RBC surface. The small difference in sizes between two immunoreactive proteins (FIG. 1F) is consistent with the calculated molecular weight of the C-terminal region (15 kDa) that is likely detached when the protein is exported. Furthermore, the observations that antibodies against PfEMMA1 fragment 2 recognize presumably cleaved and exported surface proteins (FIG. 2, FIG. 3, FIG. 4 and FIG. 5) whereas anti-C-terminus antibodies do not label such proteins (FIG. 5) support our hypothesis.

### Example 5: PfEMMA1 is not Essential for In Vitro Parasite Survival, but Potentiates Growth Inhibition by Anti-PfEMMA1 Antibodies

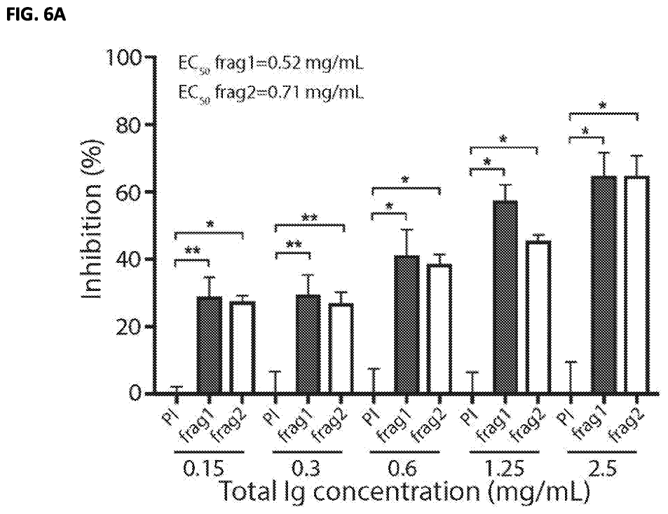

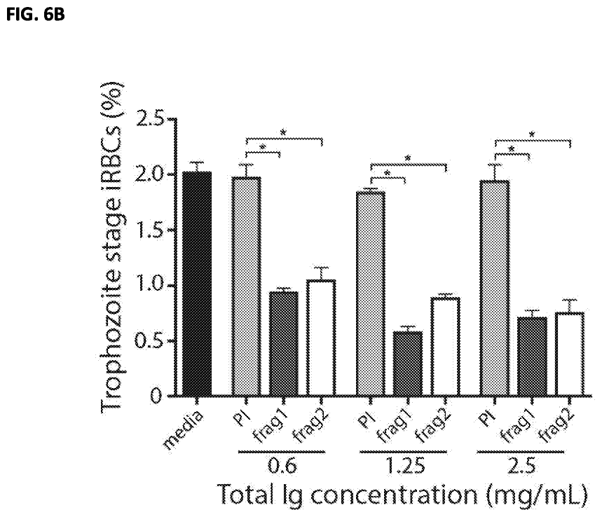

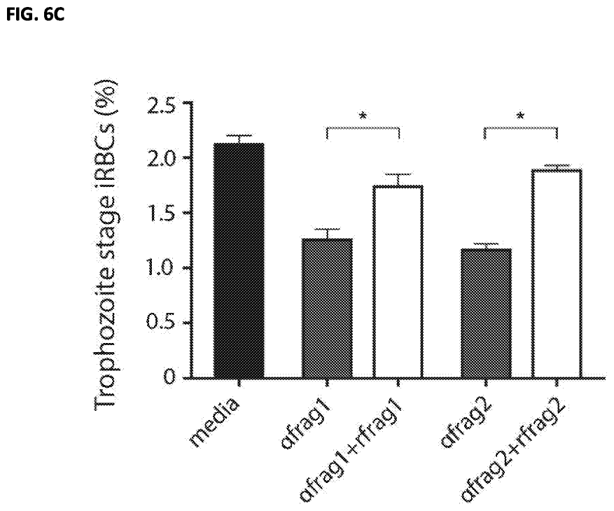

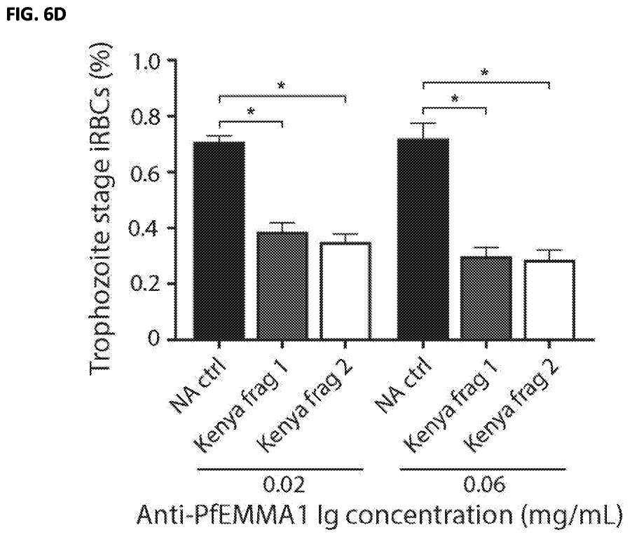

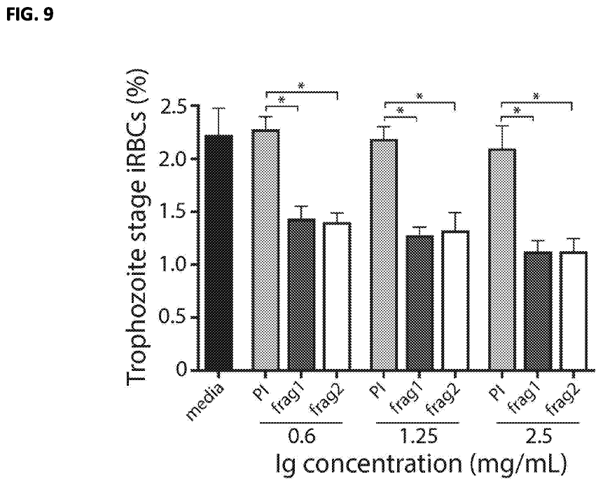

While PfEMMA1 function appears to be dispensable for parasite growth in vitro, studies were carried out to determine whether it could mediate antibody-dependent inhibition of parasite growth/invasion. Growth/invasion inhibition assays (GIA) were performed using total polyclonal immunoglobulins (Ig) purified from mice immunized with rPfEMMA1 fragments (strain 3D7 sequences) to determine if there is an in vitro correlate of protection. Three heterologous Pf strains (3D7, Dd2 and W2) were cultured with anti-rPfEMMA1 or pre-immune antibodies. 3D7 parasite growth was inhibited by 40-68% compared with control antibodies in a dose-dependent manner (all P≤0.005; FIG. 6A), which is similar to results published for GIA using viral vector-induced rabbit IgG against full-length PfRH5, the leading blood-stage vaccine candidate. In contrast to this observation for PfEMMA1, PfRH5-derived subunit vaccines failed to show GIA protection (A. D. Douglas et al., Nat Commun 2, 601 (2011)). GIA with PfDd2 (FIG. 6B) and PfW2 (FIG. 9) strains showed inhibition up to 68% and 47%, respectively. The concentration of total Ig necessary to induce 50% growth inhibitory activity (EC50) for anti-PfEMMA1 fragments 1 and 2 against 3D7 was estimated to be 0.52 mg/mL and 0.71 mg/mL, respectively (FIG. 6A). The inhibitory effect of Ig was confirmed to be attributable to anti-PfEMMA1-specific antibodies by demonstrating a substantial competitive neutralization of Ig by rPfEMMA1 (P<0.003; FIG. 6C). Concordantly, affinity-purified anti-PfEMMA1 Ig from immune Kenyan adults resulted in up to 60% growth inhibition (FIG. 6D).

### Example 6: Immunization with rPbEMMA1 Protects Mice Against Lethal PbANKA Challenge

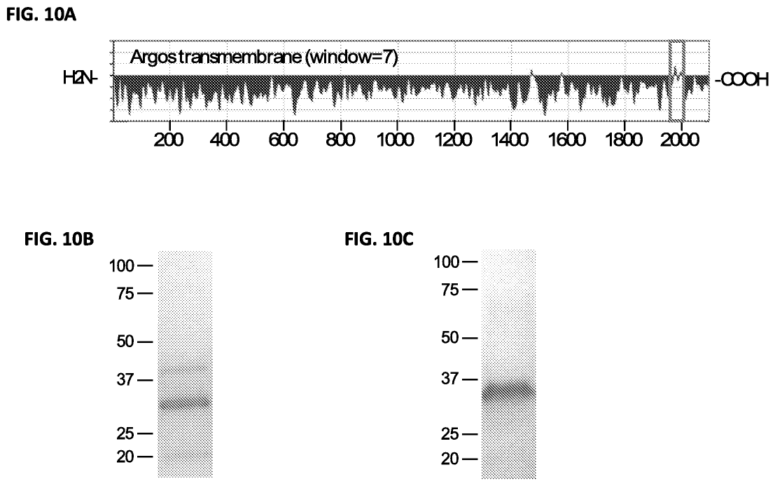

PbEMMA1 (PBANKA_0914100) and PfEMMA1 have 26.5% amino acid identity (57.2% similarity), and identities of fragment 1 and 2 orthologs are 23.4% and 22.0%, respectively. Their overall protein architecture is similar, with a predicted transmembrane domain near the C-terminus (FIG. 10A). Like PfEMMA1, the gene for PbEMMA1 is not essential (E. Bushell et al., Cell 170, 260-272 e268 (2017)), and the recombinant PbEMMA1 (rPbEMMA1) fragments 1 and 2 were successfully expressed and purified using FPLC (FIG. 10B, FIG. 10C).

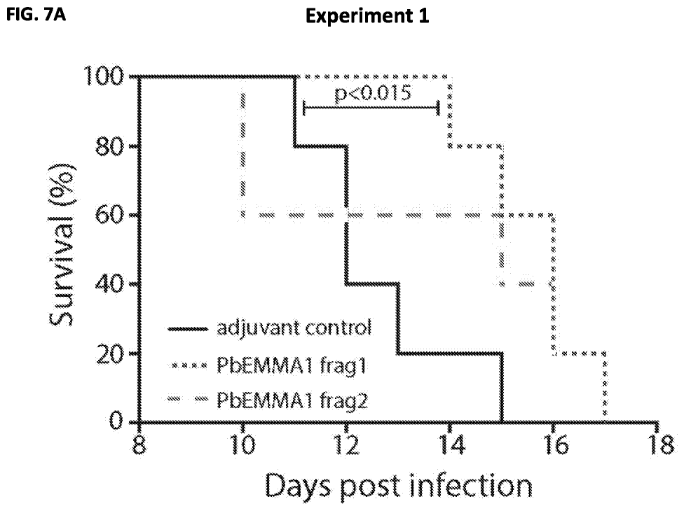

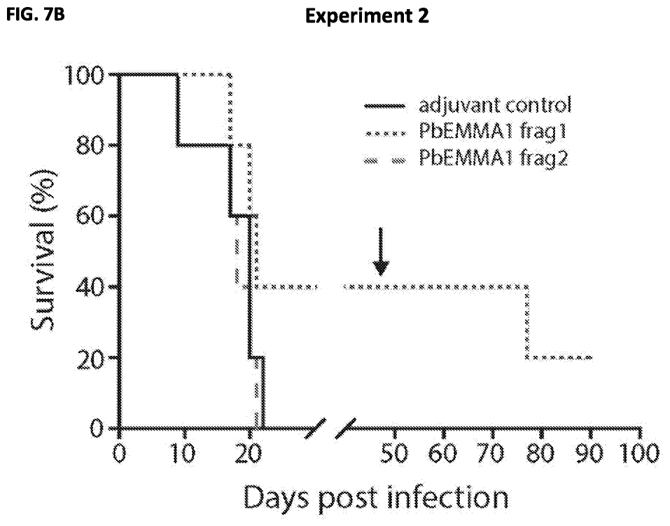

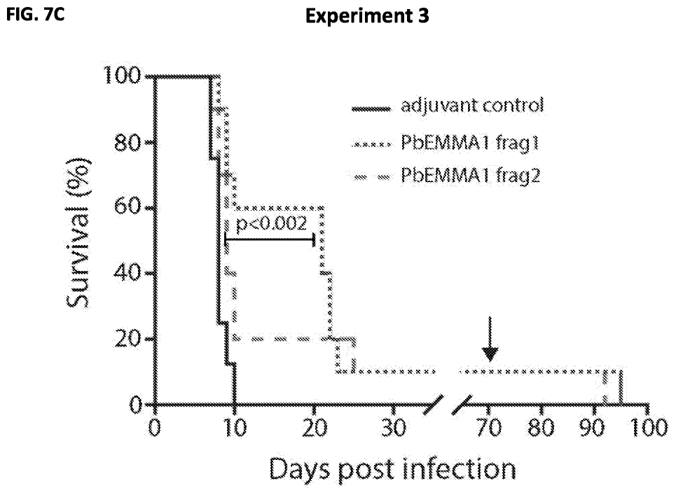

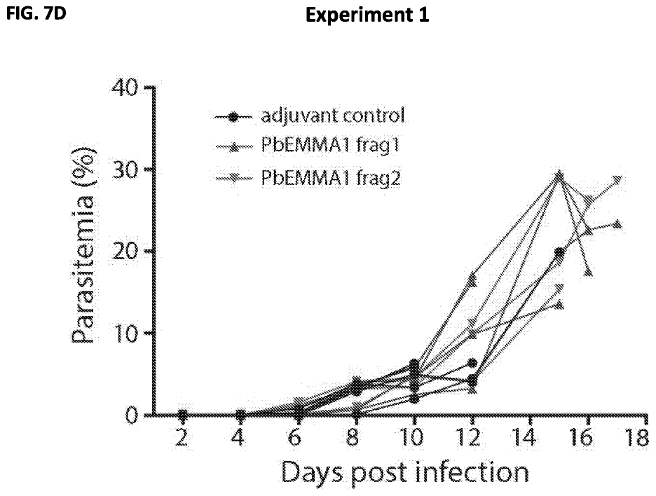

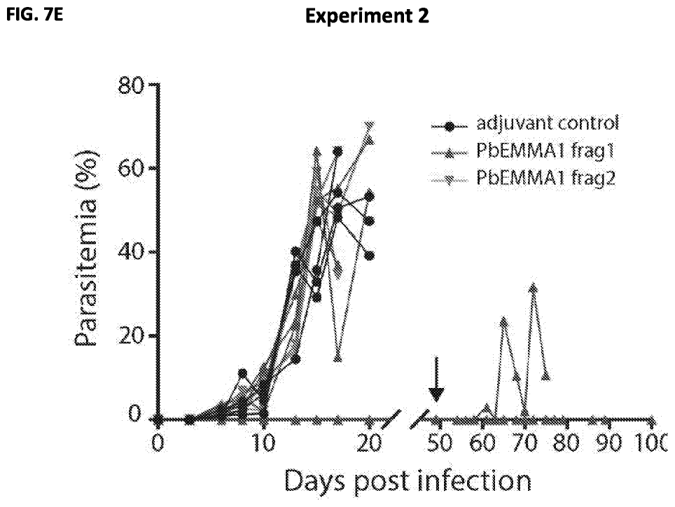

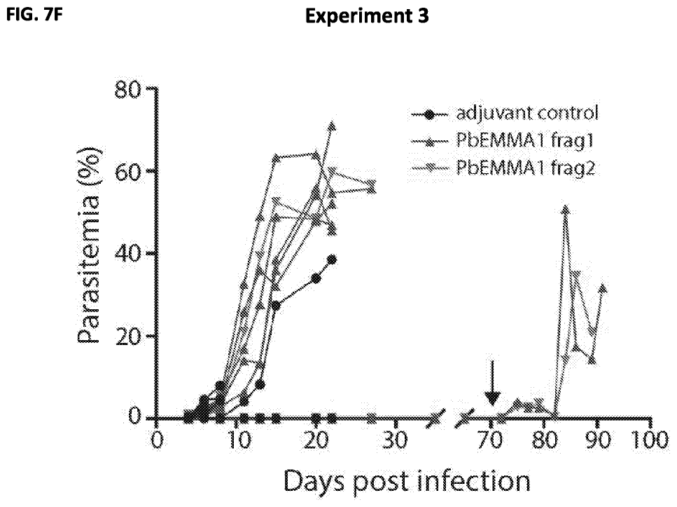

The effectiveness of immunization with rPbEMMA1 to protect BALB/cJ mice against lethal challenge with PbANKA was evaluated. This model in general recapitulates several aspects of severe malaria in humans (A. L. Goodman et al., Sci Rep 3, 1706 (2013)), and provides a stringent test of the protective efficacy of immunization with rPbEMMA1. Three immunization trials were conducted with a total of 58 mice with different inoculum sizes. Mice generated exuberant anti-rPbEMMA1 IgG responses to a biweekly series of immunizations with rPbEMMA1 adjuvanted with TiterMax Gold (geometric mean endpoint titers ≥1:256,000 mean fluorescence intensity [MFI] units) as compared with adjuvant alone (below lower limit of detection). In the first trial (FIG. 7A), BALB/cJ mice were immunized three times via intraperitoneal route (i.p.) before challenge with 10⁴ PbANKA-infected RBCs also administered i.p. rPbEMMA1 fragment 1-immunized mice survived 1.3 times longer than controls (P<0.015).

To enhance efficacy and prevent peritoneal inflammation, four immunizations via the subcutaneous (s.c.) route were used for subsequent experiments. In the second trial (FIG. 7B), immunized BALB/cJ mice were challenged i.p. with 10⁴ P. berghei ANKA pRBCs. Two mice (40%) immunized with rPbEMMA1 fragment 1 (each with antibody titers of 1:512,000 MFI units) had low-density parasitemia (2-4 parasites/10,000 RBCs) on the third day after inoculation, and subsequently completely eradicated parasites and remained healthy for the duration of the study. The remaining mice succumbed within 22 days in a similar timeframe as control mice. To investigate durability of immunity after immunizations and parasite exposure, the two surviving mice were rechallenged on day 49 with the same inoculum as before. Two healthy unimmunized BALB/cJ mice were inoculated concurrently to serve as infected controls. All mice had documented low-density parasitemia three days later. Progressive parasitemia led to death in both controls (12 and 18 days later) and one immunized mouse (28 days later), whereas the other mouse promptly eradicated the parasites and survived until the end of the study (day 100).

In the third trial (FIG. 7C), immunized BALB/cJ mice were challenged i.p. with a 5-fold higher inoculum (5×10⁴ PbANKA pRBCs) to test a more rapidly progressive disease model. All mice had initial parasitemia documented by microcopy. All adjuvant controls died by day 10 (median, day 8), whereas 6 of 10 (60%) mice immunized with rPbEMMA1 fragment 1 survived beyond 10 days (median day of death, 21; P<0.002; FIG. 7C). One mouse (10%) from each group of mice immunized with rPbEMMA1 fragments 1 or 2 (each with antibody titers of 1:2,048,000 MFI units) eradicated parasitemia by day 4. These two surviving mice were rechallenged with 10⁴ PbANKA pRBCs on day 70 after initial challenge, and survived for an additional 25 and 22 days, respectively, compared with two concurrently infected unimmunized controls that died on days 8 and 10

Mice immunized with rPbEMMA1 fragment 1 (n=17 from the 3 experiments) had high parasite densities (assessed with light microscopy) for a significantly longer period than mice immunized with rPbEMMA1 fragment 2 (p<0.002; n=19) or adjuvant alone (p<0.001, n=18), indicating that immunization with rPbEMMA1 fragment 1 conferred a significant survival advantage to mice despite high circulating parasite densities (FIG. 7D, FIG. 7E and FIG. 7F)

### Example 7: Natural Anti-PfEMMA1 Antibodies are Associated with Lower Parasitemia in Tanzanian Children

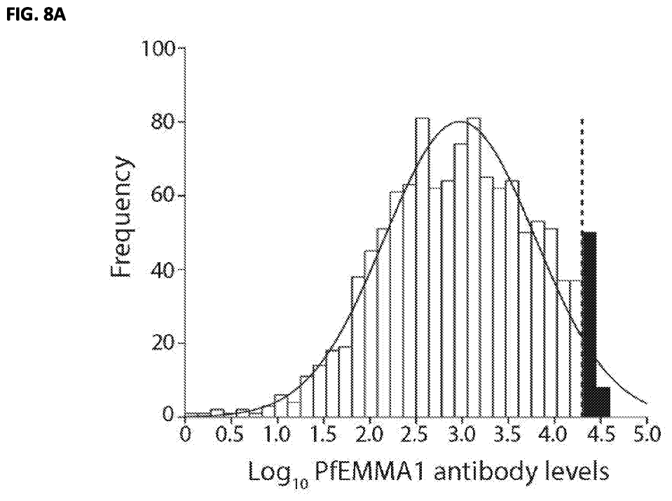

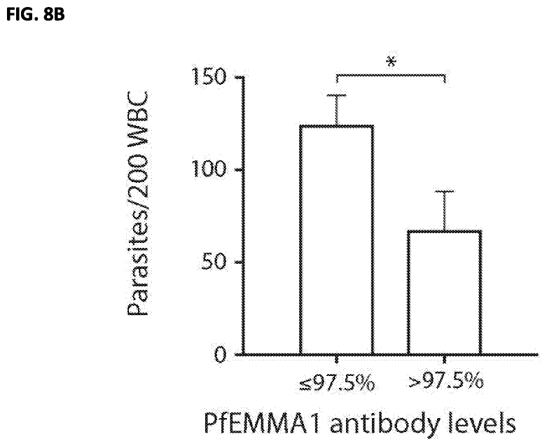

To investigate PfEMMA1 as a potential vaccine candidate, the impact of naturally acquired antibodies to PfEMMA1 on parasite levels was evaluated in children aged 48 weeks to 3.5 years enrolled in a Tanzanian birth cohort (B. P. Goncalves et al., N Engl J Med 370, 1799-1808 (2014)). Anti-PfEMMA1 fragment 1 and 2 IgG levels were measured using a fluorescent bead-based assay, and related these to subsequent parasitemia. A total of 1,274 antibody measurements in plasma was obtained from 540 children at scheduled and sick visits. The average time interval between each antibody measurement and either a subsequent antibody determination or completion of the study was 28.1 weeks. Subjects were followed for a total of 32,064 child-weeks of observation. For the following analyses, various thresholds of antibody levels were used to dichotomize the data (50th, 75th, 90th and 97.5th percentiles). It was found that antibody levels above only the highest cutoff value (97.5th percentile) predicted protection against parasitemia, a phenomenon which has been considered important for other vaccine candidates (K. J. Ewer et al., Vaccine 33, 7444-7451 (2015), F. H. Osier et al., Sci Transl Med 6, 247ra102 (2014)). It was observed that a slightly higher frequency of children had antibody levels above this value (FIG. 8A). The frequency distribution of antibody levels >97.5% percentile in children at various ages was as follows: 48 weeks (n=6); 76 weeks (n=18), 100 weeks (n=16), 124 weeks (n=8) and 148 weeks (n=3). In total, there were 14,722 points of contact with children (scheduled and sick visits). Last observation carried forward (LOCF) antibody and parasite density levels were derived from all points of contact. Blood smears were obtained from 51 children at 646 scheduled or sick visits during the defined periods when their anti-PfEMMA1 fragment 1 levels were >97.5 percentile, and from 535 children at 14,076 visits during periods when their antibody levels were 97.5 percentile.

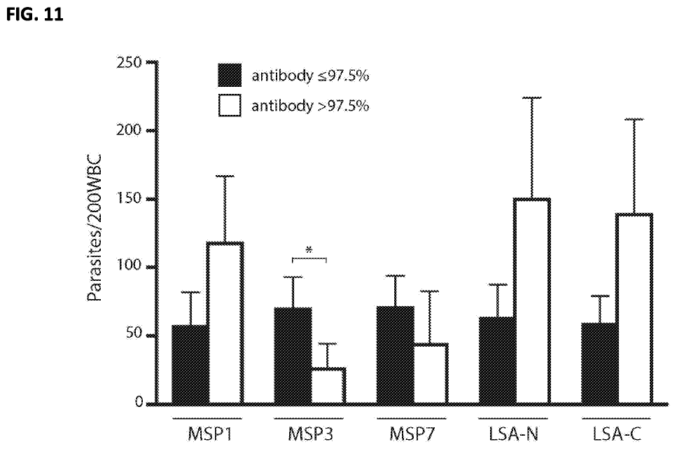

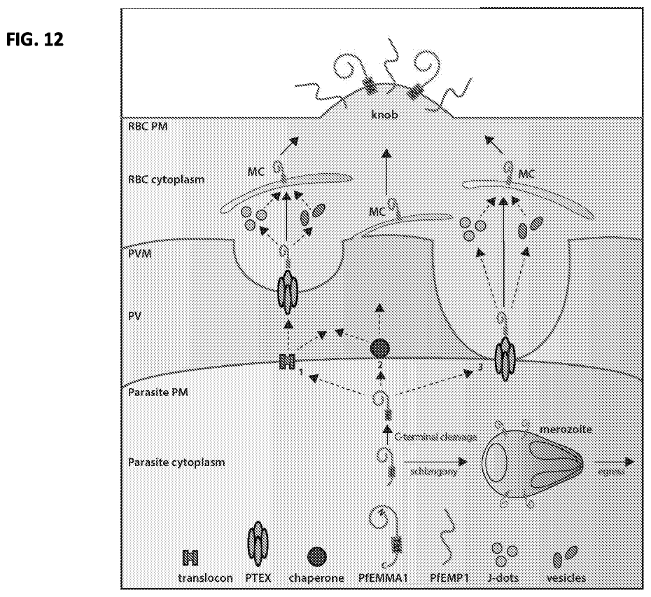

In generalized estimating equation (GEE) repeated-measures modeling, children with anti-PfEMMA1 fragment 1 antibody levels >97.5 percentile had a 46% reduction in parasite densities compared with those with lower antibody levels (rate ratio (RR)=0.54, 95% CI, 0.30-0.97, P=0.038; FIG. 8B) after adjusting for age, scheduled or sick visits, sickle cell trait, bed net usage, placental malaria and season at birth. There were no associations with parasite density when lower antibody level cutoffs (median, 75th and 90th percentiles) were used. In the same model, sickle cell trait (RR=0.54; 95% CI, 0.37-0.77, P<0.001) and bed net usage (RR=0.53; 0.38-0.75, P<0.001) had similar effect sizes, and are similar to previously reported hazard ratios in the same population (B. P. Goncalves et al., N engl J Med 370, 1799-1808 (2014)). No association with parasite density was found for anti-PfEMMA1 fragment 2 antibodies. For comparison, available antibody responses to other recombinant falciparum proteins tested (MSP1, MSP7, LSA) did not predict resistance to parasitemia using the same statistical method and dichotomized outcome as for PfEMMA1 (FIG. 11). However, antibodies to MSP3, an established vaccine candidate (WHO, Geneva, Switzerland (2017), P. Druilhe et al., PLoS Med 2, e344 (2005)), were associated with reduced parasite density (RR=0.38, 95% CI, 0.15-1.0, P=0.05) using the same statistical approaches as for PfEMMA1. GEE modeling did not reveal a significant protective effect of high anti-PfEMMA1 antibody levels against severe malaria. However, the small number of children with the relevant predictor (antibody level >97.5 percentile; n=51) and outcome (severe malaria; n=53) limited the power of this analysis. FIG. 12 shows a model of export of malaria proteins (C. Gruring et al., Cell Host Microbe 12, 717-729 (2012), J. A. Boddey and A. F. Cowman Annu Rev Microbiol 67, 243-269 (2013), N. J. Spillman, et al., Annu Rev Biochem 84, 813-841 (2015), T. Spielmann and T. W. Gilberger Trends Parasitol 31, 514-525 (2015), T. Spielmann and T. W. Gilberger Trends Parasitol 26, 6-10 (2010), J. M. Przyborski, et al., Mol Microbiol 101, 1-11 (2016), A. Heiber et al., PLoS Pathog 9, e1003546 (2013), and M. Marti and T. Spielmann, Curr Opin Microbiol 16, 445-451 (2013)) that illustrates the two distinct trafficking pathways of PfEMMA1.

### Example 8: A Highly Conserved Blood-Stage Malaria Antigen on the Surface of Erythrocytes and Merozoites Induces Protective Antibodies

PF3D7_1134300, a single copy gene with few polymorphisms, was previously identified as a target of antibodies in plasma from resistant, but not susceptible Tanzanian children (D. K. Raj et al., Science 344, 871-877 (2014)). The encoded protein, PfEMMA1, is expressed in blood stage parasites and is dually localized to the RBC and merozoite surfaces. PfEMMA1 appears to be dispensable for parasite growth in vitro. However, anti-PfEMMA1 antibodies (murine- and human-derived) specifically inhibit parasite growth by up to 68% in vitro, and PfEMMA1 expression is absolutely required for mediating this effect.

The PbANKA/BALB-cJ mouse model of malaria was used to test the protective efficacy of PbEMMA1 immunizations in vivo. It has been shown that the PbANKA strain is more virulent than PbNK65 and PbK-173 (C. S. Toebe et al., Am J Trop Med Hyg 56, 192-199 (1997), T. Wang et al., Parasitol Res 99, 238-252 (2006), A. Wan Omar et al., Trop Biomed 24, 119-126 (2007), and T. M. Lopera-Mesa et al., Vaccine 26, 1335-1343 (2008)) and that challenges with PbANKA are uniformly lethal at 20- to 100-fold lower inocula than those used in the current model when mice immunized with AMA1, MSP1₄₂, or MSP9 antigens were infected (A. L. Goodman et al., Sci Rep 3, 1706 (2013)). Although the identity of Pb- and PfEMMA1 is only ˜23% at the amino acid level, self-cure or prolonged survival was demonstrated in a significant number of mice immunized with an adjuvanted polypeptide derived from PbEMMA1, named fragment 1. It was found that immunizing mice via the s.c. route confers greater protection compared with i.p. route. Forty percent (2/5) of immunized mice infected with 10⁴ parasites had self-resolving parasitemia and one of these mice survived rechallenge with the same inoculum, which could reflect boosted natural immunity after Pb exposure and/or a durable vaccine-induced protective response. Similarly, of the ten immunized mice challenged with a five-fold higher inoculum, 60% of immunized mice had more than double the median survival time of controls (21 versus 8 days, respectively) and 10% of immunized mice from the same trial effected self-cure.

Although rPbEMMA1 fragment 1-immunized mice survived longer than controls after challenge with parasites, mice with persistent parasitemia had unexpectedly high densities that persisted significantly longer than that of control mice. It was concluded that the immune responses in these mice conferred a significant survival advantage despite extremely high circulating parasite levels. The imaging studies of P. falciparum revealed that the protein is expressed on the surface of infected RBCs. As P. falciparum and P. berghei EMMA1 both have predicted transmembrane domains that may facilitate protein export and association with RBC membranes, surface exposure of PbEMMA1 may afford clinical protection by altering host-pathogen interactions (e.g. reducing cytoadherence, similar to Pfvap1 (A. Nacer et al., Cell Microbiol 17, 1205-1216 (2015)), reducing deleterious cytokine production by innate immune cells, and/or blocking RBC invasion (T. King et al., PLoS Pathog 11, e1005118 (2015), H. A. Lagasse et al., J Leukoc Biol 99, 659-671 (2016), B. Franke-Fayard et al., PLoS Pathog 6, e1001032 (2010), F. El-Assaad et al., Infect Immun 81, 3984-3991 (2013), B. G. Yipp et al., Blood 101, 331-337 (2003)).

To investigate potential protection mediated by anti-PfEMMA1 antibodies in humans, a longitudinal cohort of Tanzanian children between the ages of 48 weeks and 3.5 years, who had a wide range of naturally acquired anti-PfEMMA1 antibodies, was analyzed. An almost 50% reduction in parasitemia among children with >97.5th percentile of antibody concentrations against a particular domain was observed. This requirement for high antibody levels is consistent with the notion that for subunit vaccines to be effective in humans, they may need to induce immune responses of very high magnitudes that often exceed those stimulated by natural exposure to the same antigens (K. J. Ewer et al., Vaccine 33, 7444-7451 (2015), F. H. Osier et al., Sci Transl Med 6, 247ra102 (2014)). The same statistical model used to analyze the protective effects of PfEMMA1, also predict that high-level antibodies against MSP3, a blood-stage vaccine candidate undergoing clinical trials, sickle cell trait, the strongest known natural protective factor, and bed net usage (WHO, Geneva, Switzerland (2017), P. Druilhe et al., PLoS Med 2, e344 (2005)) were all associated with significantly reduced parasitemia. On the other hand, the observed differences in immunoprotection mediated by two adjacent fragments of PfEMMA1 likely arise from distinct antigenic and/or functional properties of these sequences. It was confirmed that native PfEMMA1 is immunogenic and induces a range of antibody levels in Tanzanian children, albeit predominantly below the protective threshold. Therefore, natural boosting following immunization would help ensure a durable vaccine-induced response.

PfEMMA1 is uniquely differentiated from several other surface exposed proteins including the variant surface antigens (VSA) because it is encoded by a single copy gene with low polymorphism, whereas VSAs comprise five highly polymorphic multigene families: var (60 genes) that encode PfEMP1 (L. Hviid et al., Adv Parasitol 88, 51-84 (2015)); surf (10 genes) that encode SURFINs (G. Winter et al., J Exp Med 201, 1853-1863 (2005)); rif (150-200 genes) that encode RIFINs (N. Joannin et al., BMC Genomics 9, 19 (2008)); stevor (39 genes) that encode STEVOR (M. Niang et al., Cell Host Microbe 16, 81-93 (2014)); and Pfmc-2tm (13 genes) that encode P. falciparum Maurer's cleft 2 transmembrane proteins (C. Lavazec et al., Mol Microbiol 64, 1621-1634 (2007)). Two other integral surface proteins, Clag3.1 and Clag3.2, also have substantial diversity and are encoded by almost identical paralogous rhoptry genes that are expressed mutually exclusively in complex with RhopH1. These complexes form part of the plasmodial surface anion complex (PSAC)/new permeability pathway (NPP) that permits solute and nutrient acquisition by intracellular parasites (W. Nguitragool et al., Cell 145, 665-677 (2011)). Notably, Clag3.1 and Clag3.2 have extracellular loops that are as highly polymorphic as EBA-175 and AMA1 (microneme proteins), which are under diversifying selection forces imposed by host immune pressure (W. Nguitragool et al., Cell 145, 665-677 (2011), H. Iriko et al., Mol Biochem Parasitol 158, 11-21 (2008)).

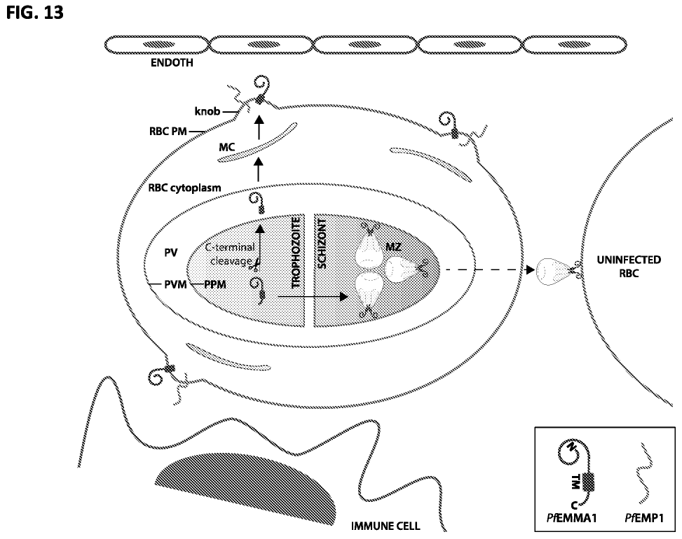

The processing and export pathway of PfEMMA1 in human pRBCs was delineated. Different immunofluorescence staining patterns with antibodies that recognize portions of the protein either upstream (N-terminus side) or downstream (C-terminus side) of the predicted transmembrane region indicate that there are 2 distinct destinations of processed protein that are summarized in FIG. 13: 1) full length PfEMMA1 is retained within the parasite plasma membrane and incorporated into the developing merozoites, where it is expressed on the surface, or 2) a truncated form of PfEMMA1 lacking the C-terminus segment, is trafficked out of the parasite via Maurer's clefts to the surface of RBCs. PfEMMA1 is expressed on the surface of only ˜5% of non-permeabilized RBCs infected with late-stage parasites as detected by flow cytometry. PfEMMA1's ectodomain comprises the segment of protein to the N-terminal side of the transmembrane domain, which is similar to that of PfEMP1, the majority of which is located on the inner aspect of the RBC membrane (J. G. Waterkeyn et al., EMBO J 19, 2813-2823 (2000), T. S. Rask et al., PLoS Comput Biol 6, (2010)) Immuno-transmission electron microscopy confirmed that PfEMMA1 clusters on electrodense knobs in non-permeabilized pRBCs, similar to PfEMP1, which is the major virulence factor that mediates cytoadhesion (J. A. Chan et al., Cell Mol Life Sci 71, 3633-3657 (2014)). Transmembrane domains of PNEPs also present in PfEMP1 may mediate protein export (M. Marti et al., Curr Opin Microbiol 16, 445-451 (2013)); the hydrophobic α-helical transmembrane domain and possibly a flanking region in the case of PfEMMA1 may subsume the functions of a signal peptide and PEXEL-motif, and anchor the protein to Maurer's clefts and RBC plasma membranes (T. Spielmann et al., Trends Parasitol 26, 6-10 (2010)). However, unlike many other exported proteins with or without PEXEL motifs, PfEMMA1 is not encoded by a gene in the subtelomeric region (A. Heiber et al., PLoS Pathog 9, e1003546 (2013)), nor does it contain conserved N-terminus sequences described in some other PNEPs (T. J. Sargeant et al., Genome Biol 7, R12 (2006)).

PfEMMA1 is characterized by low frequency of polymorphisms in field isolates from Africa despite being expressed on the exofacial surface of pRBC and merozoites. PfRH5 and other single copy proteins exposed on the RBC surface (PIESP2 and PfJ23) have relatively low sequence diversity (S. K. Nilsson Bark et al., Mol Cell Proteomics (2017), K. S. Reddy et al., Proceedings of the National Academy of Sciences of the United States of America 112, 1179-1184 (2015)). Specifically, PfRH5 is a merozoite surface antigen that is highly susceptible to neutralizing antibodies, but is unlikely to be under substantial immune selection pressure based on the fact that there is a lack of intrinsic immune recognition in Africans. This observation indicates that the parasite has evolved an immunomodulatory mechanism to protect merozoites during the critical step of RBC invasion (A. D. Douglas et al., Nat Commun 2, 601 (2011)). PfEMMA1 may have limited sequence variation for various reasons, including restricted display and/or immune recognition on the RBC exofacial surface. This is consistent with data showing that ˜5% of infected RBCs are detected with labeled PfEMMA1 antibodies. Membrane association of PfEMMA1 in cytoadherence-mediating, knob structures on the infected RBC surface, and interactions with other proteins in these structures may contribute to limiting immune accessibility. PfEMMA1 may be subdominant, may be protected by other surface proteins that serve as immune decoys, or may interact directly with host immune cells leading to immunomodulation as shown for some other proteins (A. Nacer et al., Cell Microbiol 17, 1205-1216 (2015), G. J. Wright et al., PLoS Pathog 10, e1003943 (2014)).

All blood stage vaccines currently in clinical development target only merozoite antigens (WHO, Geneva, Switzerland (2017)). PfEMMA1 represents a unique class of vaccine antigen, because it is the only highly conserved malaria protein known to be localized on the surface of both RBCs and merozoites. The multigene STEVOR family is also localized on both of these surfaces, although distinct STEVOR variants appear to express different proteins at each location (A. Khattab et al., Malar J 10, 58 (2011)). Nevertheless, STEVOR proteins are not viable vaccine candidates because they display considerable diversity (M. Niang et al., Cell Host Microbe 16, 81-93 (2014)). This double “Achilles' heel” could potentially be susceptible to vaccine-induced antibody blockade for prolonged periods (hours) rather than only brief windows (seconds to minutes) during which merozoite-specific targets are accessible during each replication cycle. Mechanisms of protection by anti-PfEMMA1 antibodies can include: (1) blockade of merozoite invasion of RBCs and/or 2) disruption of interactions between RBCs and host cells. PfEMMA1 serves critical function(s) as part of a surface virulence complex that could modulate cytoadherence and/or antigen display on the infected RBC surface to increase susceptibility to immune responses.

The findings described herein support the use of PfEMMA1 as a vaccine against malaria. PfEMMA1 vaccines, together with other conserved immunogenic malaria vaccine candidates, may provide synergistic, highly effective and strain-transcending immunity against the most dangerous species causing human malaria.

### Materials and Methods

The following materials and methods were used to generate the data described herein.

### Reagents

All reagents were obtained from Sigma-Aldrich unless stated otherwise.

### Parasite Population Genetics

Genome-wide VCF files containing variant calls for Pf samples collected in Senegal and Malawi were downloaded from the Pf3k project, release 5; malariagen.net/projects/Pf3k, M. Manske et al., Nature 487, 375-379 (2012).

Analyses were limited to single nucleotide polymorphisms (SNPs) that fell within coding regions of PfEMMA1 and other blood-stage vaccine candidates, passed all Pf3K filters, including the GATK VQSLOD filter, and contained only single-clone lineages of Pf. Variants were annotated as synonymous or nonsynonymous using the provided SnpEff calls. To identify single-clone infections, low-quality genes (≥25% heterozygous calls or missing calls) were first masked. Then, samples with genome-wide variant sites containing ≥2% heterozygous calls or ≥4% missing calls were removed from further analysis. Genes with either 20% heterozygous calls or missing calls in the remaining samples were then masked. Within each sample, any remaining heterozygous calls were transformed into homozygous calls by retaining the allele with highest read support (A. M. Early et al., Nat Commun 9, 1381 (2018)). This filtering provided a set of 99 Senegal and 110 Malawi samples. For each gene, custom Perl scripts were used to calculate pairwise nucleotide diversity (n), Tajima's D, and Weir and Cockerham's estimate of F_{ST} (B. S. Weir et al., Evolution 38, 1358-1370 (1984)). To exclude genes with poor coverage, downstream analyses only included genes that included at least 5 SNPs.

### Recombinant PfEMMA1 and PbEMMA1 Expression and Purification

Codon-optimized PfEMMA1 ORFs encoding aa 1164-1401 (Pf fragment 1), aa 1364-1600 (Pf fragment 2), aa 2140-2223 (C-terminus) and PbEMMA1 ORFs encoding aa 1141-1365 (Pb fragment 1) and aa 1343-1575 (Pb fragment 2) were cloned into pD451 (ATUM) except for Pf fragment 2 that was cloned into pET30 (Novagen, EMD Millipore). Pf fragments 1 and 2 partially overlap each other as well as the PfEMMA1 polypeptide present in the original Pf3D7 cDNALambda Zap library. The plasmids encode a fused Stag (amino side) and 10× His tags (carboxy side), to facilitate identification and metal chelate chromatography, respectively. The resulting plasmids were transformed into E. coli BL21(DE3)(Novagen, EMD Millipore). Transformants were cultivated in 10 L batches of Terrific broth with 100 μg/mL kanamycin as previously described (D. K. Raj et al., Science 344, 871-877 (2014)). Fifty grams of cell paste was dissolved in phosphate-buffered saline (PBS), 1% Triton X-100 and 100 mM phenylmethylsulfonyl fluoride. Cells were lysed by high-pressure disruption at 20,000 PSI (Microfluidics, Model 110-T) and the lysate was then incubated with NP-40 at 4° C. for 30 minutes. Inclusion bodies contained in the pellet were resuspended in PBS using a tissue homogenizer and disrupted under high pressure as before. The resulting pellet was dissolved in buffer containing 8 M urea, 10 mM potassium phosphate, 300 mM NaCl, 10 mM imidazole.

Recombinant proteins were purified under endotoxin-free conditions using a 2-step process on BioPilot chromatography equipment (Pharmacia). First, the dissolved pellet was applied to an AP-1 column (Waters) containing 12 mL of Nuvia IMAC nickel-charged resin (Bio-Rad) and protein was refolded on-column by exchanging buffer containing urea with urea-free buffer over 10 column volumes. Bound protein was eluted with a stepped gradient containing increasing concentrations of imidazole. The fractions containing the protein of interest were pooled and then further purified by anion exchange chromatography using an UNO Q12 column (Bio-Rad). Proteins were eluted with a linear gradient of elution buffer (1 M NaCl, 10 mmol/L Tris, 1 mmol/L EDTA [pH 8.0]) and buffer exchanged into 50 mM Na3PO4 for storage at −80° C., under which conditions proteins were stable for at least one year. The identities of the recombinant polypeptides were confirmed by immunoblots and liquid chromatography-tandem mass spectrometry (W.M. Foundation Biotechnology Resource Laboratory). Protein concentrations were measured with a BCA assay kit (Pierce). MSP1 (19 kDa region of 3D7 strain from BEI Resources/MR4), MSP3 (aa 99-265), MSP7 (aa 117-248), LSA-N (aa 28-150) and LSA-C (aa 1630-1909) were purified as previously described (D. K. Raj et al., Science 344, 871-877 (2014)). Proteins used for immunizations contained less endotoxin than the threshold pyrogenic dose of 5 EU/kg measured with a chromogenic LAL endotoxin assay that conforms to FDA standards (ToxinSensor, GenScript).

### Parasite Strains and Culture

Pf strains representing sialic acid independent (3D7) and sialic acid dependent (W2 and Dd2) RBC invasion pathways, and PbANKA were obtained from BEI Resources/MR4. Pf strains were cultivated in vitro as previously described (D. K. Raj et al., Science 344, 871-877 (2014), W. Trager et al., Science 193, 673-675 (1976)). Blood smears were prepared and culture medium was exchanged every 48 hours.

### Anti-PfEMMA1 Immunoglobulins

Mouse anti-PfEMMA1 antisera were generated by immunizing BALB/cJ (JAX) mice with 50 μg of rPfEMMA1 fragments emulsified with equal volumes of TiterMax Gold adjuvant (CytRx Corp.) subcutaneously at two-week intervals for four doses Immune sera were preadsorbed with fresh uninfected human O+ RBCs in a 20:1 volume ratio for 1 hour to remove any anti-human RBC Ig's, and heat inactivated at 56° C. for 30 min to remove complement. Total Ig G, A and M were purified using sequential precipitation with caprylic acid and ammonium sulfate (CA-AS) as previously described (F. Perosa et al., J Immunol Methods 128, 9-16 (1990), E. S. Bergmann-Leitner et al., Malar J 7, 129 (2008)) and dialyzed against RPMI 1640 media (Gibco) in spin columns. To affinity purify specific human anti-PfEMMA1 antibodies, we coupled 3 mg of rPfEMMA1 to 1 ml of NHS activated Sepharose 4 Fast Flow chromatography resin (GE Health Sciences) according to the manufacturer's instructions. Coupled resin was incubated with 3 mL of pooled human plasma (preadsorbed with uninfected human O⁺ RBCs) that we obtained from healthy, HIV-negative, non-pregnant Kenyan adults who were not receiving antimalarial therapy and who participated in a research study in Bondi), Western Kenya as previously described (A. E. Frosch et al., J Immunol 198, 4629-4638 (2017)). After extensive washing in PBS and 0.05% Tween 20, bound antibody was eluted in 0.1M glycine, pH 2.3 and immediately neutralized with 1M Tris HCl pH 9.0. Eluted antibodies were dialyzed against RPMI 1640 media in spin columns (Amicon Ultra-15, EMD Millipore) and sterilized prior to use (Ultrafree-MC, 0.22 μm pore, EMD Millipore). Antibody concentrations were measured with a NanoDrop 2000c and confirmed with a BCA protein assay (Thermo Scientific).

### Western Blots

Western blots were performed using standard protocols, e.g., as previously described (D. K. Raj et al., Science 344, 871-877 (2014)). Lysates of RBCs that were uninfected or infected with trophozoite- and schizont-stage parasites were prepared. Proteins were resolved by SDS-PAGE on a 4-15% polyacrylamide gel (Bio-Rad) and stained with GelCode Blue Stain Reagent (Thermo Scientific). For western blots, mouse polyclonal anti-PfEMMA1 antisera or pre-immune sera were used at a 1:750 dilution.

### Growth Inhibition Assays (GIA)

GIA were conducted with varying concentrations of CA-AS-purified total Ig's (e.g., as described in E. M. Malkin et al., Infect Immun 73, 3677-3685 (2005)) compared with media and pre-immune sera controls. Sorbitol synchronized trophozoite-stage Pf parasites (C. Lambros et al., J Parasitol 65, 418-420 (1979)) at approximately 0.4% parasitemia and 1% final hematocrit were incubated with sera/Ig's in a final volume of 50 μL in microtiter wells for 40 hours (one replication cycle). Cultures were performed in triplicate with three biological replicates. Blood films were stained with Giemsa and microscopists blinded to treatment conditions enumerated trophozoite-stage pRBCs among at least 2000 RBCs per slide. EC50 was calculated using non-linear regression. To test the effect of neutralization of antibodies on growth inhibition, anti-fragment 1 and 2 immunoglobulins (2.5 mg/mL each) were preincubated with recombinant fragment 1 and 2 proteins, respectively (650 nM each) at room temperature for one hour.

### Mouse Immunization Regimens and Antibody Assays

Groups of 6-8 week old female BALB/cJ mice were immunized two weekly with 50 μg recombinant proteins and equal volume of TiterMax Gold (CytRx Corp.) via three i.p. or four s.c. injections. Antibody assays were performed with rPbEMMA1 fragment 1- and 2-coated Bio-Plex COOH beads (Bio-Rad) e.g., as described in D. K. Raj et al., Science 344, 871-877 (2014). The endpoint titer of serially diluted mouse sera was determined two weeks after final immunization prior to parasite challenge to determine antibody concentrations. Mice were challenged with 1-5×10⁴ PbANKA-infected RBCs and were monitored with blood films daily from day 2 to 5 post-challenge and then on alternate days to quantify parasitemia. Mice exhibiting signs of cerebral malaria (seizure or paresis) or excessive weight loss were euthanized in accordance with the approved animal protocol.

### Immunofluorescence Assays

Blood smears of asynchronous Pf3D7 strain parasite cultures were permeabilized and fixed with 100% methanol at −20° C. for 15 minutes and blocked with PBS/2% BSA. Permeabilized pRBCs were probed with mouse anti-PfEMMA1 antisera (1:250), mouse pre-immune sera (1:250), rabbit anti-AMA1 and anti-RH5 (1:500; gifts of Robin Draper), rabbit anti-REX1 and -SBP1 (1:2000 and 1:5000, respectively; gifts of Tobias Spielmann), rabbit-anti-GPC (1:500; Abcam), rabbit anti-MSP1 and -4 (1:500; MR4). Blood smears were incubated with primary antibodies for 2 hours at 25° C., washed in PBS, and incubated with goat anti-mouse IgG conjugated with Alexa Fluor 488 (Invitrogen) and goat anti-rabbit IgG conjugated with Alexa Fluor 594 (Invitrogen) for 1 hour at 25° C. After washing with PBS, pRBCs were mounted onto glass slides with Vectashield Antifade Mounting Medium (Vector Laboratories) containing 4′,6′-diamino-2-phenylindole (DAPI) to label nuclei.

To evaluate surface localization in non-permeabilized pRBCs, live cell staining was performed on sorbitol-synchronized late-stage Pf3D7-infected RBCs that were enriched with LS columns and a QuadroMACS separator (Miltenyi Biotec). 10⁸ live pRBCs were blocked for 1 hour at 4° C. in PBS/2% BSA. Non-permeabilized pRBCs were incubated with anti-PfEMMA1 mouse antisera (1:15) or pre-immune sera (1:15) and anti-MSP4 rabbit polyclonal antibodies (1:15; Abcam) in PBS/2% BSA for 2 hours at 4° C. After washing with PBS, samples were incubated with secondary antibodies at 4° C. as described above. Washed cells were resuspended in PBS and blood smears were fixed with 100% methanol for 15 minutes at −20° C. Slides were covered with Vectashield (Vector Laboratories) antifade mounting medium containing DAPI to label nuclei. Slides were imaged with a Nikon C1si confocal microscope (Nikon Inc. Melville, N.Y.) using diode lasers 402, 488 and 561. Sequential Z series sections of pRBC were collected at 0.1 μm with a 100× Plan Apo and a scan zoom of 4. Analyses were performed on deconvolved, 3D acquisitions with Nikon's Elements software (v3.2). In each Z stack, regions of interest were outlined and analyzed with Nikon's colocalization macro. Pearson's Correlation Coefficient >0.5 was considered indicative of colocalization.

### Flow Cytometry

Sorbitol synchronized predominantly schizont-stage Pf3D7-infected human O+ RBCs were enriched with LS columns and a QuadroMACS separator (Miltenyi Biotec) to >80% purity by light microscopy. Washed live non-permeabilized cells were prepared at 1% hematocrit in 100 μL Hank's Balanced Salt Solution (HBSS, Gibco). Samples of infected or uninfected RBCs were predigested with 1 mg/mL of trypsin or chymotrypsin in PBS with 10% sucrose for 15 minutes at 37° C. to remove surface glycoproteins. Digestion was stopped with 1 mg/mL of soy trypsin inhibitor. Washed samples of digested or non-digested, infected or uninfected cells were then incubated with preadsorbed mouse anti-PfEMMA1 (1:15 HBSS) or pre-immune sera (1:15 HBSS) in addition to 14 μM Hoechst 33342 and 0.35 μM MitoTracker (Life Technologies) for 30 minutes at 37° C. to stain parasitic DNA and mitochondria, respectively. Washed cells were incubated with anti-mouse IgG1-FITC (Miltenyi Biotec) for 10 minutes at 4° C. Cells were resuspended in HBSS with 2% fetal bovine serum and 150,000 cells were analyzed for each condition with a MACSQuant Analyzer 10 (Miltenyi Biotec) flow cytometer. Data were analyzed using FlowJo software (TreeStar, Inc.). For quantification of RBCs with FITC+PfEMMA1 surface localization, only Hoechst+MitoTracker+live pRBCs were selected and values were adjusted using pre-immune sera controls.

### Immuno-Transmission Electron Microscopy

Mixed trophozoite- and schizont-stage Pf3D7-infected human O+ RBCs were enriched with LS columns and a QuadroMACS separator (Miltenyi Biotec) to >80% purity by light microscopy. Aliquots of 10⁸ live pRBCs were blocked for 1 hour at 4° C. in 1×PBS containing 2% BSA. A sample of pRBCs was permeabilized by treatment with 0.1 μg equinatoxin II (EqtII; gift of Gregor Anderluh) for 6 minutes at 25° C. (K. E. Jackson et al., Biochem J 403, 167-175 (2007)) and then washed with PBS. EqtII is a pore-forming toxin that lyses RBC membranes but not Maurer's clefts or PV membranes, releases cytoplasmic contents including hemoglobin, and permits penetration of antibodies for immunogold labeling. Samples of permeabilized or intact pRBCs were incubated with anti-PfEMMA1 fragment 2 mouse antisera (1:15) and anti-glycophorin C rabbit polyclonal antibodies (1:15; Abcam) in PBS and 0.1% BSA-c (Aurion) for 1 hour at 4° C. After washing with PBS, pRBCs were incubated with 6 nm gold-conjugated anti-mouse IgG and 10 nm gold-conjugated anti-rabbit IgG (Aurion) in PBS and 0.1% BSA-c for 1 hour at 4° C. Washed cell pellets were layered with 2% glutaraldehyde and 1% paraformaldehyde in 0.1M sodium cacodylate buffer, and allowed to fix for one week at 4° C. Cell pellets were post-fixed for 40 minutes with 0.5% osmium tetroxide, buffer rinsed, dehydrated in a graded acetone series, infiltrated and embedded in Spurr's epoxy resin. The embedded pellets were cut from the resin filled microfuge tubes and attached to blank epoxy blocks. Ultra-thin sections (50-60 nm) were retrieved onto 300 mesh copper grids and contrasted with uranyl acetate. Sections were examined at 80 kV using a CM-10 electron microscope (FEI, Hillsboro, Oreg.). Images were obtained with a model 785 Erlangshen ES1000W CCD camera (Gatan, Pleasanton, Calif.).

### Immunoepidemiological Analysis and Statistics

Subjects were enrolled in the Mother Offspring Malaria Studies (MOMS) project, which was based at Muheza Designated District Hospital (DDH), in northeastern Tanzania. Details of the MOMS study design, enrollment, methods, case definition of disease severity, and exclusion criteria have been reported, e.g., D. K. Raj et al., Science 344, 871-877 (2014). A multivariable GEE model with a gamma distribution was used to assess the impact of anti-PfEMMA1 antibody concentrations (exposure) on malaria parasite burden and clinical severity (outcomes) in Tanzanian children from 48 weeks to 3.5 years of age. Children in the original sample used for the Pf3D7 cDNA phage screen were excluded from these analyses (D. K. Raj et al., Science 344, 871-877 (2014)). Blood for antibody testing was obtained at scheduled visits approximately every 6 months. Blood smears and clinical assessments were scheduled every 4 weeks and were also performed at unscheduled sick visits (village-health worker visits, walk-in visits, hospitalizations). GEE was selected because it takes into account within-subject correlations between repeated observations in a longitudinal study comprising all available data. The outcomes for each subject were analyzed after measuring an antibody level and before the subsequent antibody test in an iterative process, using the last observation carried forward (LOCF) method. Antibody concentrations were dichotomized at 97.5% of the frequency distribution. The robust covariance matrix estimator and exchangeable correlation structure, which provided the lowest Quasilikelihood Information Criteria (QIC) level for the full model was selected; the analysis with first-order autoregressive, m-dependent, independent and unstructured correlation structures produced similar results. in addition to assessing the main effects of anti-PfEMMA1 fragments 1 and 2 antibody levels, several potential confounders were evaluated including age, birth weight, bed net usage, hemoglobin concentration, hemoglobin phenotype, parity, placental malaria, prematurity, scheduled or sick visit, and season at time of birth. A quadratic term for age (age²) was also included in the model because there appeared to be a curvilinear relationship between age and parasitemia.

Differences in survival of groups of mice were analyzed using the Kaplan-Meier log-rank test. Differences in parasitemia density assessed by GIA were analyzed using the Student's t test. A 2-tailed P<0.05 was considered to be statistically significant. Statistical analyses were performed with SAS 9.3, SPSS 24.0 and Prism 7 software.

PfEMMA1 Frag 1:
(SEQ ID NO: 27)
GACAATGTCAATAACAATAACAACAAAGAGTCCTGTGACAACATCAAGCAC
ATGCGCACGAAAAGCCTGAACTTTGTGAGCCGTGAGAGCTATGGTGAGCAT
AAGAGCCTGGACGTCTACCAGGAATGCTACGTGAAAAACAATAAGCTGATT
AACAAAGTTAACGATAAGAAATACGAAGATAACAACAATTCCTATTTGAAC
GAAGATGACAATGCGTCGATGCAGTTCTATGAAGAGACTAACAGCAATCCG
TACATCGTTGACCAAGAGAACAATATGAAAAACTACGTGAATAACGTGCTG
TACAATAACAATTCTAATTACTACGTTGATTCCAAGAATTATGACAAAAGC
AAAGAAAATGCCGAGAACAAGAGCGACGACATCCTGAATAACGAAAACATT
CACACCCTGAAAGATCAGAAGAAAAAGATCCAGAACAATAACGAATTCATT
AGCGAGCAAGCGGACATCGAGAATATCCGTAATAGCCAAGAAGAGGTTTAT
GAGAAAGAACATGAGCCGCTGTGGGTGATTAATGCTAGCAACGAAGAGAAG
AAAAGCTACGAAGAATTGATTTACAGCGATATGAGCAGCAACCGCGTCACG
AAGAATAAGTATAGCGATATGAATAACGTTGAAGTCCTGCTGAATGAGGAC
AATCTGCTGACCACCGAGAAGTATAAAGTTCAGCTGGAGAAAGAAAACAAG
PfEMMA 1 C-terminus:
(SEQ ID NO: 28)
CTGAAGATTAACAAAAAAGTTCCGAAGTATAAACTGGAGAACATGAATATC
AATAGCCCGAACATTCCGTATACCTACTTGTTTATCTGTGACGGTTCCAAT
TATCTGTGCATCAACGACAACAGCCTGAATAACGAAGTGTATGAGAACAAG
ATGAAGCTGAACAATATCATTGGCTACTACCATTACATTAACCTGAATCGC
CTGACGTACTATCTGGAAAAAGTTAACGCCAATTTCGTCTACAATCAC
PbEMMA1 Frag 1:
(SEQ ID NO: 29)
ACCAATAGCTATACGAACAACATGCTGATTAATGATTGTAACAATCTGCAG
AACAAGAGCCATCGCTATATCCGTGCGAAGTCCTTGAATGCGCTGCGCGAC
AGCATTCTGCACAGCAAATCTTTAAAGGTCTACGAAGATTGCTGCATCAAG
AACAAAAATGAAAACATCACGAATCACGACTACAATATGAAAGACATCTAC
AAGCGTTGCAGCTACATCGGTAATGAAAACACCCATGAAATCTTCTGCACC
GAGAAATCTGAGATCTGTGAGAACGATCTGAACGGTTATAACATTAAACCG
GTGCTGATTAAGTCCCACGCCGATGAAACGAATGAAAAGACCAATAACATT
CTGCTGAATGACATCAACAGCCAGATTGCACACAAGAAAAATGACAATAAC
GAAAACATTTACTTGCTGGAGAAAGAGAACAAGATTGAGCAAATCCTGACC
AACCTGTCCAATAATGAAAACGATAGCGACAATTGTACCAGCGTGACGATT
CAGCACTACACCGTTAACAATGCCGACAACAATAAAGTCGGCGATCACGTT
CTGTCGAGCTACAACATTTGCGAGCAAGAAGAGAGCCTGTGGATGCCGAAT
CAGTATAAGATCGAGAAAGCGATCCTGAAAGAGAGCAGCGAGAACTATATT
CATAAAGAGGAA
PbEMMA1 Frag 2:
(SEQ ID NO: 30)
CCGAACCAGTATAAGATTGAGAAAGCAATCCTGAAAGAGAGCTCCGAGAAT
TACATTCATAAGGAAGAATTCCAGAACGGTCTGGTGAAGTGCCGTAACAAC
TTCATCGATAATGGTATCAACCAATGCGAAGATATGCTGAGCGGCGTTATT
GAGAAAAAAGAAAATACCAATTGTAAGATTAATGACGACGAGAAAAAGAAT
TTCAACGATAAAGTTGACCAGAATGATAAAGAAATGATTGACATCTGCGTG
AACAGCTCTAGCGAGAACTGCTACATCAAGAATAACAGCAACAATCACAAC
AACAAGTATAGCATTGAAAAAGAATCCAAGAATCTGAAAGATGTCGCCCAC
GACGACTTTGAGCACACCACGATGACCACCTTTACGATTATCAACCCGAAC
ATCATTACCAGCTCTACCTTCGAGAATAATGCATCGACGTGTGAGATGATT
AAGAACTACAATCCGAATAAAGAGTGCCGCAACGTCGAGATCAATAACGAC
GCGAAAAAAGAAAAAGAAAAATACGACAATAAGAAAAAGATCACTAAGAGC
ACCAATAACAGCAGCAAGATCGGCAAAAACAATAAGCTGTGTAAGCTGAAA
AAAGCGAGCATTACGAAGAAAAACTGGAAGAACCGTGAGATTGGTGAGCTG
AGCGAGAATGAAATCTACAAGCTGCCGAGCTATTTG

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank, NCBI, and Plasmodb submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed:

1. A vaccine for preventing or reducing the severity of malaria comprising a composition that comprises a purified polypeptide comprising an amino acid sequence of SEQ ID NO: 1, 3, 5 or an antigenic fragment thereof or a polynucleotide encoding said polypeptide, wherein said polypeptide is less than 641 amino acids in length.

2. The vaccine of claim 1, wherein said polypeptide is less than 250 amino acids in length.

3. The vaccine of claim 1, wherein said polypeptide is less than 100 amino acids in length.

4. The vaccine of claim 1, wherein said polypeptide is less than 50 amino acids in length.

5. The vaccine of claim 1, wherein said polypeptide consists essentially of an amino acid sequence of SEQ ID NO: 1, 3, 5 or an antigenic fragment thereof.

6. The vaccine of claim 1, wherein said polynucleotide comprises a nucleic acid sequence of SEQ ID NO: 2, 4, 6 or a fragment thereof.

7. The vaccine of claim 1, wherein said composition inhibits parasite invasion and/or growth of parasites in red blood cells.

8. The vaccine of claim 1, wherein said antigenic fragment comprises an amino acid sequence of SEQ ID NO: 7, 8, 9, 10, 11, 12, 13, 14, 15, 16, 17, 18, 19 or 20.

9. A vaccine for preventing or reducing the severity of malaria comprising a purified monoclonal antibody that binds to a polypeptide with an amino acid sequences of SEQ ID NO: 1, 3, or 5.

10. The vaccine of claim 1, further comprising an adjuvant.

11. The vaccine of claim 1, further comprising a pharmaceutically-acceptable excipient.

12. A method of treating or preventing malaria in a subject, comprising administering to said subject the vaccine of claim 1, wherein said vaccine elicits an anti-PfEMMA1 antibody response

13. The method of claim 12, wherein said parasite invasion of red blood cells or parasite growth is inhibited in said subject.

14. The method of claim 12, wherein number of gametocytes is reduced in said subject.

15. The method of claim 12, wherein transmission of malaria is reduced in said subject.

16. The method of claim 12, wherein said vaccine is administered over a period of 8 weeks.

17. The method of claim 12, wherein said vaccine is administered annually.

18. The method of claim 16, wherein said subject is a pregnant female.

19. The method of claim 16, wherein said subject is an infant or child.

20. The method of claim 18, wherein said infant is greater than 6 weeks of age.
